(19) United States
(12) Patent Application Publication
(10) Pub. No.: US 2004/0171003 A1
(43) Pub. Date: Sep. 2, 2004

(54) Cancer-associated genes

(76) Inventors: Yoshie Yoshikawa, Kyoto (JP); Sachiko Okamoto, Kyoto (JP); Tomonori Oura, Kyoto (JP); Junichi Mineno, Kyoto (JP); Kiyozo Asada, Shiga (JP); Hiroshi Inoue, Oita (JP); Masaki Mori, Oitama (JP)

Correspondence Address: BIRCH STEWART KOLASCH & BIRCH, PO BOX 747, FALLS CHURCH, VA, 22040-0747, US

(21) Appl. No.: 10/474,495

(22) PCT Filed: Mar. 28, 2002
(86) PCT No.: PCT/JP02/03038

(30) Foreign Application Priority Data
Apr. 16, 2001 (JP) 2001-112039
Sep. 21, 2001 (JP) 2001-290193

(51) Int. Cl. C12Q 1/68

(52) U.S. Cl. 435/6

(57) ABSTRACT

To provide a rapid, simple, easy and more accurate detection of a cancer or the like, and its application to a treatment having a high specificity. Cancer-associated genes of SEQ ID NOs: 1 to 264; a method for detecting a cancer on the basis of expression of the cancer-associated genes; an array based on the cancer-associated genes; a kit for detecting a cancer; and a method for suppressing proliferation of a cancer cell.

### TECHNICAL FIELD

The present invention relates to a nucleic acid having a sequence of a cancer-associated gene of which expression is altered due to canceration by which a cancer, especially gastric cancer, can be detected, a method for detecting a cancer on the basis of the alternation of expression of the gene, a kit used therefor, and a method for suppressing proliferation of a cancer cell.

### BACKGROUND ART

Cancer has been the number one cause of death in Japan since 1981, among which gastric cancer occurs at the highest incidence. In recent years, the existence of the mechanism for multistage carcinogenesis in which a normal cell transforms to a cancer has been known [Fearon, E. R. et al., Cell, 61, 759-767 (1990); Sugimura, T., Science, 258, 603-607(1992)]. Accumulation of a plural aberrations of genes, including, for instance, those in DNA repair genes, tumor suppressor genes, oncogenes and the like are required. In general, it is considered that gene instability and inactivation of tumor suppressor gene are involved in the oncogenesis, and activation of oncogene and/or overexpression of growth factor are involved in progress of cancer and transformation to malignant cancer.

In the above instability of the genes, there are: (1) instability of genes associated with aberrations in a DNA mismatch repair system and (2) instability on a chromosomal level. Examples of the above (1) include, for instance, a difference in strand length of a simple repeat sequence existing in the genome between a cancer-affected portion and a non-affected portion of the same individual (microsatellite instability) [Thibodeau, S. N. et al., Science, 260, 816-819 (1993)] and the like. Also, examples of the above (2) include, for instance, interchromosomal rearrangement and the like.

The above interchromosomal rearrangement may result in expression of a protein not found in a normal cell, or even if it is a protein expressed in a normal cell, the rearrangement may affect its expression level. In fact, in human chronic myelocytic leukemia, it has been confirmed that fusion of bcr gene and c-abl gene is caused by interchromosomal rearrangement, thereby resulting in expression of a hybrid mRNA transcribed from the bcr-abl fusion gene, which does not exist in a normal cell. Furthermore, it has been confirmed that leukemogenesis takes place when the bcr-abl fusion gene is introduced into an animal [Authored by Watson, J. D. et al., Recombinant DNA, 2nd Edition, Maruzen Co. LTD., 309 (1992)].

The above inactivation of tumor suppressor gene includes, for instance, inactivation of p53 gene. The causation of the inactivation is considered to be induced by deletion in the gene or a point mutation in a particular portion of the encoding region [Nigro, J. M. et al., Nature, 342, 705-708 (1989); Malkin, D. et al., Science, 250, 1233-1238 (1990)]. In addition, deletions and point mutations in the p53 gene have been observed in numerous kinds of cancers. For instance, in gastric cancer, these deletions or point mutations are observed in 60% or more of cases with early cancer [Yokozaki, H. et al., Journal of Cancer Research and Clinical Oncology, 119, 67-70 (1992)]. Therefore, it is considered that detection of these mutations can be applied to early detection of cancer.

On the other hand, the p16/MTS1 gene has been known as a gene inactivated by a homo-deletion, and the homo-deletion is observed in the gene at high incidence in glioma, pancreatic cancer, urinary bladder cancer or the like [Cairns, P. et al., Nature Genetics, 11, 210-212 (1995)]. The p16 protein regulates the cell cycle, and aberrations in expression of the p16 gene have been suggested to be involved in canceration of a cell [Okamoto, A. et al., Proceedings of the National Academy of Sciences of the United States of America, 91, 11045-11049 (1994)].

In the activation of the above oncogene, causative factors therefor include, for instance, a viral insertion mutation in the vicinity of the oncogene and interchromosomal rearrangement. One example of the above viral insertion mutation includes a viral insertion mutation in chicken lymphoma caused by avian leukosis virus (ALV). In this case, it has been confirmed that ALV DNA is inserted in the vicinity of c-myc, one kind of a gene, so that normal c-myc is overexpressed or a mutant having a new sequence partially differing from the normal gene is expressed by potent enhancer and promoter of the inserted ALV. Also, as an example of the above interchromosomal rearrangement, it has been confirmed in a certain kind of human B cell tumor that c-myc, one of oncogenes, is placed under the control of the potent transcriptional signal of an immunoglobulin gene, and its expression level is increased. In this case, the difference of a c-myc expression product in a cancer cell from that expressed in a normal cell is not found on the sequence level, so that the canceration is considered to be caused by an increase in the expression level of c-myc [Authored by Watson, J. D. et al., Recombinant DNA, 2nd Edition, Maruzen Co., LTD., 305-308 (1992)].

The overexpression of growth factor includes, for instance, the overexpression of C-Met, which encodes a hepatocyte growth factor receptor. As to this expression abnormality of C-Met, there is found expression of mRNA having a length of 6.0 kilobases in length, not found in the normal mucous membrane, from an initial stage of gastric carcinogenesis [Kuniyasu, H. et al., International Journal of Cancer, 55, 72-75 (1993)], and the abnormal expression is observed in high incidence. Therefore, there is confirmed a correlation between the increase in gene copy number and cancer malignancy [Kuniyasu, H. et al., Biochemical and Biophysical Research Communications, 189, 227-232 (1992)].

As to the correlation between aberrations of genes and cancer malignancy, in addition to the above c-Met, there have been respectively confirmed an increase in copy number of C-erbB2 gene, one of oncogenes, and/or overexpression in breast cancer, ovarian cancer, gastric cancer, uterine cancer or the like [Wright, C. et al., Cancer Research, 49, 2087-2090 (1989); Saffari, B. et al., Cancer Research, 55, 5693-5698 (1995)], and an increase in copy number of K-sam gene, one of oncogenes, and/or its overexpression in poorly differentiated adenocarcinoma, one of tissue types of gastric cancer [Authored by Tahara, E. et al., Gastric Cancer, Tokyo, Springer-Verlag, published 1993, 209-217].

However, since the oncogenetic mechanism is multistage and requires the accumulation of a plural mutations, there are yet many unknown portions regarding genes associated with canceration, so that further studies are necessary.

### DISCLOSURE OF INVENTION

An object of the present invention is purposed to provide a means for applications to cancers, especially cancers in the digestive organ such as gastric cancer, esophageal cancer and colorectal cancer, concretely to applications of a rapid, convenient, and more accurate detection, treatment with high specificity, and the like. Concretely, a first object of the present invention is to provide a method for detecting a cancer, especially a cancer cell, by which the cancer can be detected rapidly, conveniently, and more accurately. A second object of the present invention is to provide a DNA array suitable for the above method for detecting a cancer, especially a method for detecting a cancer cell. A third object of the present invention is to provide a kit suitable for the above method for detecting a cancer, especially a method for detecting a cancer cell. A fourth object of the present invention is to provide a nucleic acid comprising a sequence of a cancer-associated gene of which expression, especially expression level, is altered in a cancer cell as compared to that in a normal cell. A fifth object of the present invention is to provide a method for suppressing proliferation of a cancer cell using a specific binding substance to the above cancer-associated gene or a polypeptide encoded thereby.

Concretely, the present invention relates to:

[1] a method for detecting a cancer, characterized in that the method comprises evaluating alteration of expression of at least one kind of gene selected from the group consisting of:

(a) a gene comprising a nucleic acid having either a nucleotide sequence selected from the group consisting of SEQ ID NOs: 1 to 264 or a nucleotide sequence complementary thereto, wherein an expression level of the gene in a cancer cell is altered as compared to that in a normal cell;

(b) a gene comprising a nucleic acid capable of hybridizing to a nucleic acid having either a nucleotide sequence selected from the group consisting of SEQ ID NOs: 1 to 264 or a nucleotide sequence complementary thereto under stringent conditions, wherein an expression level of the gene in a cancer cell is altered as compared to that in a normal cell; and

(c) a gene of a nucleic acid having a nucleotide sequence having at least 80% sequence identity to either a nucleotide sequence selected from the group consisting of SEQ ID NOs: 1 to 264 or a nucleotide sequence complementary thereto, wherein an expression level of the gene in a cancer cell is altered as compared to that in a normal cell;

[2] a DNA array for detecting a cancer according to the method of the above [1], wherein the array comprises at least two kinds of nucleic acids selected from the group consisting of:

(A) a nucleic acid having either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell;

(B) a nucleic acid capable of hybridizing to a nucleic acid having either a nucleotide sequence selected from the group consisting of SEQ ID NOs: 1 to 264 or a nucleotide sequence complementary thereto under stringent conditions, wherein the nucleic acid comprises the sequence of a gene of which expression level in a cancer cell is altered as compared to that in a normal cell; and

(C) a nucleic acid having a nucleotide sequence having at least 80% sequence identity to either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell, or fragments thereof, each being immobilized to a given region of a support;

[3] a kit for detecting a cancer according the method as defined in the above [1], wherein the kit comprises at least one oligonucleotide consisting of a nucleic acid capable of hybridizing under stringent conditions to mRNA transcribed from a gene selected from the group consisting of:

(a) a gene comprising a nucleic acid having either a nucleotide sequence selected from the group consisting of SEQ ID NOs: 1 to 264 or a nucleotide sequence complementary thereto, wherein an expression level of the gene in a cancer cell is altered as compared to that in a normal cell;

(b) a gene comprising a nucleic acid capable of hybridizing to a nucleic acid having either a nucleotide sequence selected from the group consisting of SEQ ID NOs: 1 to 264 or a nucleotide sequence complementary thereto under stringent conditions, wherein an expression level of the gene in a cancer cell is altered as compared to that in a normal cell; and

(c) a gene of a nucleic acid having a nucleotide sequence having at least 80% sequence identity to either a nucleotide sequence selected from the group consisting of SEQ ID NOs: 1 to 264 or a nucleotide sequence complementary thereto, wherein an expression level of the gene in a cancer cell is altered as compared to that in a normal cell;

[4] a kit for detecting a cancer according the method of the above [1], wherein the kit comprises an antibody or a fragment thereof capable of binding to a protein expressed from a gene selected from the group consisting of:

(a) a gene comprising a nucleic acid having either a nucleotide sequence selected from the group consisting of SEQ ID NOs: 1 to 264 or a nucleotide sequence complementary thereto, wherein an expression level of the gene in a cancer cell is altered as compared to that in a normal cell;

(b) a gene comprising a nucleic acid capable of hybridizing to a nucleic acid having either a nucleotide sequence selected from the group consisting of SEQ ID NOs: 1 to 264 or a nucleotide sequence complementary thereto under stringent conditions, wherein an expression level of the gene in a cancer cell is altered as compared to that in a normal cell; and

(c) a gene of a nucleic acid having a nucleotide sequence having at least 80% sequence identity to either a nucleotide sequence selected from the group consisting of SEQ ID NOs: 1 to 264 or a nucleotide sequence complementary thereto, wherein an expression level of the gene in a cancer cell is altered as compared to that in a normal cell;

[5] a nucleic acid comprising a sequence of a cancer-associated gene selected from the group consisting of:

(a′) a nucleic acid having either a nucleotide sequence selected from the group consisting of SEQ ID NOs: 66 to 264 or a nucleotide sequence complementary thereto, wherein the nucleic acid comprises the sequence of a gene of which expression level in a cancer cell is altered as compared to that in a normal cell;

(b′) a nucleic acid capable of hybridizing to a nucleic acid having either a nucleotide sequence selected from the group consisting of SEQ ID NOs: 66 to 264 or a nucleotide sequence complementary thereto under stringent conditions, wherein the nucleic acid comprises the sequence of a gene of which expression level in a cancer cell is altered as compared to that in a normal cell; and

(c′) a nucleic acid having a nucleotide sequence having at least 80% sequence identity to either a nucleotide sequence selected from the group consisting of SEQ ID NOs: 66 to 264 or a nucleotide sequence complementary thereto, wherein the nucleic acid comprises the sequence of a gene of which expression level in a cancer cell is altered as compared to that in a normal cell; and

[6] a method for suppressing proliferation of a cancer cell, characterized in that the method comprises suppressing proliferation of the cancer cell using a specific binding substance capable of specifically binding to:

(1) at least one nucleic acid selected from the group consisting of:

(A) a nucleic acid having either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell;

(B) a nucleic acid capable of hybridizing to a nucleic acid having either a nucleotide sequence selected from the group consisting of SEQ ID NOs: 1 to 264 or a nucleotide sequence complementary thereto under stringent conditions, wherein the nucleic acid comprises the sequence of a gene of which expression level in a cancer cell is altered as compared to that in a normal cell; and

(C) a nucleic acid having a nucleotide sequence having at least 80% sequence identity to either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell; or

(2) a polypeptide encoded by the above (1).

### BRIEF DESCRIPTION OF THE DRAWINGS

FIG. 1 is an autoradiogram showing electrophoretic patterns of the DNA fragment obtained in a case where a cancer-associated gene is detected according to a differential display (DD) method.

### BEST MODE FOR CARRYING OUT THE INVENTION

The present invention is based on the findings of the present inventors that there exists a differentially expressed gene in a cell of a cancer tissue and a cell of a control normal tissue of an individual suffering from a cancer, concretely a cancer in the digestive organ such as gastric cancer, esophageal cancer or colorectal cancer, more concretely papillary adenocarcinoma, well differentiated adenocarcinoma, moderately differentiated adenocarcinoma, poorly differentiated adenocarcinoma, signet-ring cell carcinoma, mucinous carcinoma, or the like, and further that a cancer cell can be detected at a surprising reliability by using an expression level of the gene as an index.

The method for detecting a cancer of the present invention is a method based on evaluation of an alteration in expression of a gene that can be used as an index for canceration in a sample excised from the body, i.e., an alteration of an expression level of a gene of which expression level is altered in a cancer cell as to that in a normal cell.

According to the method for detecting a cancer of the present invention, a cancer (a cancer lesion, a cancer cell, or the like) can, for instance, be detected in a sample from an individual suffering from a cancer, concretely a cancer in the digestive organ such as gastric cancer, esophageal cancer or colorectal cancer, more concretely papillary adenocarcinoma, well differentiated adenocarcinoma, moderately differentiated adenocarcinoma, poorly differentiated adenocarcinoma, signet-ring cell carcinoma, mucinous carcinoma, or the like.

More concretely, one of the significant features of the method for detecting a cancer of the present invention resides in that a method for detecting a cancer, namely a cancer or a cancer (a cancer lesion, a cancer cell, or the like) in a sample from an individual suffering from a cancer, characterized in that the method comprises evaluating alteration of expression of at least one kind of gene selected from the group consisting of:

(a) a gene comprising a nucleic acid having either a nucleotide sequence selected from the group consisting of SEQ ID NOs: 1 to 264 or a nucleotide sequence complementary to the sequence, wherein an expression level of the gene in a cancer cell is altered as compared to that in a normal cell;

(b) a gene comprising a nucleic acid capable of hybridizing to a nucleic acid having either a nucleotide sequence selected from the group consisting of SEQ ID NOs: 1 to 264 or a nucleotide sequence complementary to the sequence under stringent conditions, wherein an expression level of the gene in a cancer cell is altered as compared to that in a normal cell; and

(c) a gene of a nucleic acid having a nucleotide sequence having at least 80% sequence identity to either a nucleotide sequence selected from the group consisting of SEQ ID NOs: 1 to 264 or a nucleotide sequence complementary to the sequence, wherein an expression level of the gene in a cancer cell is altered as compared to that in a normal cell.

The gene that can be used as an index of canceration usable in the detection method of the present invention refers to a gene of which expression, especially expression level, is altered in a cancer cell as compared to that in a normal cell, with the canceration of the cell. In other words, the gene is a gene of which expression is significantly induced or suppressed in a cancer cell as compared to that in a normal cell. In the present specification, the gene is also referred to as “cancer-associated gene.” The “gene of which expression level is altered in a cancer cell as compared to that in a normal cell” which is used in the detection method of the present invention includes a gene comprising a nucleic acid having either the nucleotide sequence shown in SEQ ID NOs: 1 to 264 mentioned below or a nucleotide sequence complementary to the sequence.

Concretely, in the present specification, the phrase “of which expression is significantly induced in a cancer cell as compared to that in a normal cell” means that the expression level is increased in a cancer cell, for instance, 1.5 folds or more, preferably 2 folds or more that in a normal cell. In addition, the phrase “of which expression is significantly suppressed in a cancer cell as compared to that in a normal cell” means that the expression level is decreased to in a cancer cell, for instance, two-thirds or less, preferably one-half or less, that in a normal cell.

The detection method of the present invention is advantageous in the detection of a cancer, especially a cancer in the digestive organ such as gastric cancer, esophageal cancer or colorectal cancer or in the detection of a cancer (cancer lesions, cancer cells, and the like) in a sample from an individual suffering from a cancer in the digestive organ such as gastric cancer, esophageal cancer or colorectal cancer. Concretely, the detection method of the present invention is advantageous in the detection of various kinds of gastric cancers, for instance, papillary adenocarcinoma, well differentiated adenocarcinoma, moderately differentiated adenocarcinoma, poorly differentiated adenocarcinoma, signet-ring cell carcinoma and mucinous carcinoma, or in the detection of samples from individuals suffering from various kinds of gastric cancers, for instance, papillary adenocarcinoma, well differentiated adenocarcinoma, moderately differentiated adenocarcinoma, poorly differentiated adenocarcinoma, signet-ring cell carcinoma and mucinous carcinoma.

In the detection method of the present invention, samples to be tested include samples excised from the body, for instance, blood, urine, feces, tissues excised by surgical means, and the like, and samples obtained by treating these, including, for instance, nucleic acid samples and the like. The sources of the samples to be tested include, but are not particularly limited to, normal individuals, individuals suspected of suffering from a disease such as cancer, and individuals suffering from a disease such as cancer.

The above “gene of which expression level is altered in a cancer cell as compared to that in a normal cell” can be detected, for instance, by analyzing the copy number of a gene on genome or chromosomal rearrangement pattern, by comparing an expression level of a gene product in a normal cell with an expression level of a gene product in a cancer cell, thereby specifying a gene product of which expression level is altered between both cells mentioned above, or the like.

The above gene product includes, for instance, mRNA transcribed from a gene, cDNA obtained from mRNA, and a protein which is a translational product.

In the detection method of the present invention, it is more advantageous to detect the above “gene of which expression level is altered in a cancer cell as compared to that in a normal cell” with mRNA as in index, for which various techniques for analysis have been developed with the advances in gene manipulation techniques, from the viewpoints of operational ease and efficiency.

The “gene of which expression level is altered in a cancer cell as compared to that in a normal cell” used in the present invention is obtained by, for instance, selecting a gene of which expression of mRNA is altered using the mRNA as an index. A means for searching a gene of which expression is altered using mRNA as an index include a subtractive hybridization method [Zimmermann, C. R. et al., Cell, 21, 709-715 (1989)]; Representational Difference Analysis (RDA) method [Lisitsyn, N. et al., Science, 259, 946-951 (1993)], a molecular index method (Japanese Patent Laid-Open No. Hei 8-322598), a differential display (DD) method [Liang, P., and Pardee, A. B., Science, 257, 967-971 (1992)]; and the like. Among the above techniques, the DD method permits easy operation and is suitable for gene screening in the present invention.

The method for obtaining a “gene of which expression level is altered in a cancer cell as compared to that in a normal cell” used in the present invention will be hereinafter described in detail for a screening method using the DD method and a method for confirming alteration in expression in the large number of candidate genes obtained, without intending to limit the genes used in the present invention to those obtained by these techniques.

First, RNA is extracted separately from a cancer tissue cell (cancer cell) to be compared and a normal control tissue cell (normal cell). Next, each of the above RNAs is treated with DNase to remove genomic DNA, thereby giving a crude RNA sample. The mRNA is converted to cDNA by a reverse transcription reaction using the above crude RNA sample, an oligo(dT) anchor primer and reverse transcriptase (RTase). Thereafter, nucleic acid amplification is carried out by a polymerase chain reaction (PCR) using a combination of an oligo(dT) anchor primer and various random primers.

Next, each of the PCR amplified product obtained from the cancer tissue and the PCR amplified product obtained from the normal control tissue is subjected to polyacrylamide gel electrophoresis for every combination of the same primer pair to compare the band patterns. From the comparison, a band of which intensity is different between the cancer cell and the normal cell, namely, the band corresponding to a product of which expression level is altered, is found. The above band is cut out from the gel, and the nucleic acids contained in the band are extracted to give a gene of which expression level is altered, namely a DNA fragment considered to be complementary to a partial region of a cancer-associated gene mRNA.

Next, whether or not alteration in expression can be confirmed in the DNA fragment obtained by the above DD method is studied. Here, in a case where expression of mRNA is higher in the normal control tissue cell (normal cell) than that of the cancer tissue cell (cancer cell), the gene corresponding to the mRNA is used as an index that the gene is a cancer-associated gene of which expression is decreased due to canceration. On the other hand, in a case where expression of mRNA is higher in the cancer tissue cell (cancer cell) than that of the normal control tissue cell (normal cell), the gene corresponding to the mRNA is used as an index that the gene is a cancer-associated gene of which expression is amplified due to canceration.

The expression level of mRNA can, for instance, be evaluated by labeling the resulting DNA fragment obtained by the above DD method according to a commonly used labeling method, carrying out Northern hybridization or RNase protection assay [Methods in Enzymology, 155, 397-415 (1987)] with crude RNA extracted from each of a cancer tissue and a normal control tissue as a sample using the labeled DNA fragment as a detection probe, and confirming the difference in signal intensity obtained. Here, it can be judged that the more intensive the signal intensity, the higher the expression of the mRNA.

When the expression levels of more than dozens of genes are evaluated, a method using a DNA array is preferred.

In the present specification, the DNA array refers to an array comprising nucleic acids each comprising sequences of genes or nucleic acids each consisting of a fragment of the gene, wherein each of the nucleic acids is immobilized to a given region of a support. The DNA array encompasses, for instance, those referred to as DNA chips. In addition, those in which a gene or a DNA fragment from the gene is immobilized to a support at a high density are also referred to as DNA microarray.

The support for the DNA array may be of any of those that can be used in hybridization, and includes slide glass, silicone chips, nitrocellulose or nylon membranes, and the like.

By the use of the DNA array as described above, there is an advantage that even when a large number of kinds of nucleic acid molecules are contained in the nucleic acid sample, amounts of the large number of kinds of nucleic acid molecules can be determined simultaneously, and an advantage that measurements can be made even with a small amount of a nucleic acid sample. For instance, the mRNAs in the sample can be simultaneously detected, and further the expression level of the mRNAs can be determined by preparing a labeled nucleic acid such as labeled mRNA obtained by labeling mRNA in the sample or a labeled cDNA obtained by labeling cDNA obtained with mRNA as a template, and carrying out hybridization between the labeled nucleic acid and a DNA array.

More concretely, mRNA is prepared from a cell of a cancer lesion tissue collected from an individual suffering from cancer, i.e., a cancer patient, and a reverse transcription reaction is carried out with the mRNA obtained as a template. In the above reverse transcription reaction, a labeled cDNA can, for instance, be obtained by using appropriately labeled primers, labeled nucleotides or the like. The labeling method includes methods using a compound containing radioisotopes, a fluorescent substance, a ligand (biotin or the like) or the like.

Next, hybridization is carried out between the above labeled cDNA and a DNA array in which each of the nucleic acids of genes used as indices of canceration or fragments thereof are immobilized.

Here, the “gene of which expression level is altered in a cancer cell as compared to that in a normal cell” used in the present invention is a gene corresponding to a DNA fragment that can be found as a band showing an intensity difference between a normal cell and a cancer cell by the DD method.

The DNA microarray can be obtained by isolating and amplifying the DNA fragment contained in the band, and immobilizing the DNA obtained to a support. According to the DNA microarray obtained, the alterations in expression can be confirmed simultaneously even when a large number of kinds of nucleic acid molecules are present. The method for isolating a DNA fragment includes TA cloning [Bio/Technology, 9, 657-663 (1991)], and procedures such as purification by agarose gel electrophoresis and cutting out.

As the nucleic acid immobilized to a support, in addition to a nucleic acid prepared by enzymatic amplification, there can be preferably used those obtained by denaturing the nucleic acid at the time of immobilization to the support for immobilizing DNA, to give a single-stranded DNA or a derivative thereof.

The method for amplifying each of nucleic acids immobilized to a support, i.e., nucleic acids each corresponding to cancer-associated genes or fragments thereof, is not particularly limited. There can be preferably utilized, for instance, the ICAN (Isothermal and Chimeric primer-initiated Amplification of Nucleic Acid) method (see WO 00/56877) and PCR method. As the primer used in amplification in the above ICAN method or PCR, there can be used universal primers located at the multicloning site, in a case where the subject to be amplified is cloned into a vector. In a case where the subject to be amplified is not cloned into a vector, a nucleotide sequence of the DNA fragment obtained by the above DD method is sequenced by PCR direct sequencing [Authored by Erlich, H. A., PCR Technology], and primers for amplification designed on the basis of its nucleotide sequence information can be used.

The above derivative of the single-stranded nucleic acid is not particularly limited, as long as the derivative is a nucleic acid modified so that the derivative can be immobilized to a surface of the support. The derivative includes, for instance, a DNA into which a functional group such as amino group or thiol group is introduced at a 5′-terminal thereof.

The above DNA array can be prepared by a known method, for instance, by immobilizing each of nucleic acids, each corresponding to the “gene(s) of which expression levels are altered in a cancer cell as compared to that in a normal cell” or a fragment thereof to a support incorporated with amino group. In addition, a DNA array to which genes are aligned and immobilized can be prepared by carrying out the above immobilization procedures by using an apparatus for preparing a DNA array, for instance, an apparatus for preparing DNA chip manufactured by Affymetrix. The DNA array is also encompassed in the present invention.

Hybridization may be carried out by a known method, and as the conditions, those suitable for the DNA array and the labeled cDNA used may be appropriately selected. For instance, there can be carried out under the conditions described in Molecular Cloning, A laboratory manual, 2nd ed., 9.52-9.55 (1989).

By comparing the hybridization results for a sample from a cancer lesion portion with the hybridization results for a control sample, genes each having different expression levels between both samples mentioned above can be detected.

Concretely, for instance, as to the array by which hybridization is carried out with a labeled nucleic acid sample, there is carried out the detection of appropriate signals according to the labeling method used, and the expression level of each gene on the array in the sample from the cancer lesion and the expression level of that in the control sample are compared. In this case, it is more preferable to use a multiple wavelength detection fluorescence analyzer by which a plurality of labels, for instance, two kinds of fluorescents, can be detected, because the difference in gene expression between the sample from the cancer lesion and the control sample can be compared on the same DNA array. For instance, the cancer lesion sample is fluorescent-labeled with Cy3-dUTP, and the control nucleic acid sample (for instance, a normal tissue, a cell of an epithelial cell line, or the like) is fluorescent-labeled with Cy5-dUTP. Next, the labeled nucleic acid sample from the cancer lesion sample and the control nucleic acid sample are mixed in an equivolume, and the resulting mixture and the DNA array are subjected to hybridization, whereby the difference in gene expression between both samples mentioned above can be detected as differences in color and fluorescence intensity.

Each of the thus obtained genes each having a significant difference in signal intensity is a gene of which expression level is altered due to canceration of a cell, that can be used as an index for canceration (cancer-associated gene).

Here, there may be some cases where an amplified DNA fragment obtained by the above DD method with mRNA as a template, which is deduced to be expressed from the cancer-associated gene, is not cDNA complementary to the full-length cancer-associated gene. The cDNA of a full-length cancer-associated gene can be obtained, for instance, by preparing a cDNA library of a tissue used for screening by an appropriate method, and carrying out screening according to hybridization with the amplified DNA fragment obtained by the above DD method as a probe, and can also be obtained according to the RACE (rapid amplification of cDNA ends) method [Proc. Natl. Acad. Sci. USA, 85, 8998-9002 (1988)].

The cancer-associated gene used in the present invention is a gene of which difference is found by the present inventors between a gastric cancer tissue and a normal control tissue according to the DD method, and further is a gene of which alteration of expression is confirmed in a plural gastric cancers according to the DNA microarray. The cancer-associated genes thus clarified include those genes that have already been isolated and sequenced by homology search using a database in which the nucleotide sequence information of the gene is recorded, but unclear as to its association with canceration. The correspondences of SEQ ID NOs for the nucleotide sequences of DNA fragments isolated by the DD method, the clone numbers containing the DNA fragments, the names and GenBank accession numbers for the DNA fragments revealed by homology search are as follows:

H. sapiens mRNA for RING protein [SEQ ID NO: 1, clone number: 20, (GenBank accession number: Y07828)];

Homo sapiens mRNA for PKU-β, complete cds [SEQ ID NO: 2, clone number: 42, (GenBank accession number: AB004885)];

Homo sapiens carboxy terminus of Hsp70-interacting protein (CHIP) mRNA, complete cds [SEQ ID NO: 3, clone number: 49, (GenBank accession number: AF129085)];

Homo sapiens mRNA; cDNA DKFZp58610523 (from clone DKFZp58610523 [SEQ ID NO: 4, clone number: 68, (GenBank accession number: AL050217)];

H. sapiens Sp17 gene [SEQ ID NO: 5, clone number: 95, (GenBank accession number: Z48570)];

Homo sapiens MD-1 mRNA, complete cds [SEQ ID NO: 6, clone number: 129, (GenBank accession number: AF057178)];

H. sapiens mRNA for BiP protein [SEQ ID NO: 7, clone number: 132, (GenBank accession number: X87949)];

Human chromosome 17q12-21 mRNA, clone pOV-2, partial cds [SEQ ID NO: 8, clone number: 155, (GenBank accession number: U18919)];

Homo sapiens dead box, X isoform (DBX) mRNA, alternative transcript 2, complete cds [SEQ. ID NO: 9, clone number: 203, (GenBank accession number: AF000982)];

Human ferritin heavy chain mRNA, complete cds [SEQ ID NO: 10, clone number: 291, (GenBank accession number: M97164)];

Human ovarian cancer downregulated myosin heavy chain homolog (Docl) mRNA, complete cds [SEQ ID NO: 11, clone number: 295, (GenBank accession number: U53445)];

Homo sapiens full length insert cDNA, clone YP42A04 mRNA [SEQ ID NO: 12, clone number: 323, (GenBank accession number: AF085884)];

Human mRNA for protein disulfide isomerase-related protein P5, complete cds [SEQ ID NO: 13, clone number: 337, (GenBank accession number: D49489)];

Homo sapiens Ku70-binding protein (KUB3) mRNA, partial cds [SEQ ID NO: 14, clone number: 344, (GenBank accession number: AF078164)];

Human nonmuscle myosin heavy chain-B (MYH10) mRNA, partial cds [SEQ ID NO: 15, clone number: 354, (GenBank accession number: M69181)];

Human voltage-dependent anion channel isoform 1 (VDAC) mRNA, complete cds [SEQ ID NO: 16, clone number: 377, (GenBank accession number: L06132)];

Homo sapiens phosphatidylinositol 4-kinase mRNA, complete cds [SEQ ID NO: 17, clone number: 382, (GenBank accession number: L36151)];

Human β adaptin mRNA, complete cds [SEQ ID NO: 18, clone number: 383, (GenBank accession number: M34175)];

Homo sapiens H β 58 homolog mRNA, complete cds [SEQ ID NO: 19, clone number: 386, (GenBank accession number: AF054179)];

Homo sapiens unknown mRNA, complete cds [SEQ ID NO: 20, clone number: 394, (GenBank accession number: AF047439)];

Homo sapiens NRD convertase mRNA, complete cds [SEQ ID NO: 21, clone number: 426, (GenBank accession number: U64898)];

H. sapiens mRNA for acylphosphatase, muscle type (MT) isoenzyme [SEQ ID NO: 22, clone number: 430, (GenBank accession number: X84195)];

Homo sapiens transmembrane protein BR1 (BR1) mRNA [SEQ ID NO: 23, clone number: 436, (GenBank accession number: AF152462)];

Homo sapiens dynactin subunit (p22) mRNA, complete cds [SEQ ID NO: 24, clone number: 449, (GenBank accession number: AF082513)];

Human mRNA for aldose reductase (EC 1.1.1.2) [SEQ ID NO: 25, clone number: 461, (GenBank accession number: X15414)];

Homo sapiens cDNA FLJ20693 fis, clone [SEQ ID NO: 26, clone number: 463, (GenBank accession number: AK000700)];

Homo sapiens mRNA for scrapie responsive protein 1 [SEQ ID NO: 27, clone number: 503, (GenBank accession number: AJ224677)];

Homo sapiens KIAA0402 mRNA, partial cds [SEQ ID NO: 28, clone number: 507, (GenBank accession number: AB007862)];

Human transactivator protein (CREB) mRNA, complete cds [SEQ ID NO: 29, clone number: 511, (GenBank accession number: M27691)];

Human MAL protein gene mRNA, complete cds [SEQ ID NO: 30, clone number: 522, (GenBank accession number: M15800)];

Homo sapiens ataxin-2-like protein A 2LP (A2LG) mRNA, complete cds [SEQ ID NO: 31, clone number: 552, (GenBank accession number: AF034373)];

Human phosphatidylinositol transfer protein mRNA, complete cds [SEQ ID NO: 32, clone number: 556, (GenBank accession number: M73704)];

H. sapiens mRNA for transketolase [SEQ ID NO: 33, clone number: 561, (GenBank accession number: X67688];

Homo sapiens nibrin (NBS) mRNA, complete cds [SEQ ID NO: 34, clone number: 584, (GenBank accession number: AF051334)];

Human SnRNP core protein Sm D3 mRNA, complete cds [SEQ ID NO: 35, clone number: 589, (GenBank accession number: U15009)];

Homo sapiens FUS/TLS protein gene mRNA [SEQ ID NO: 36, clone number: 602, (GenBank accession number: AF071213)];

Homo sapiens, clone 486790 diphosphoinositol polyphosphate phosphohydrolase mRNA, complete cds [SEQ ID NO: 37, clone number: 609, (GenBank accession number: AF062529)];

Human globin gene. mRNA [SEQ ID NO: 38, clone number: 629, (GenBank accession number: M69023)];

H. sapiens mRNA for aminopeptidase P-like [SEQ ID NO: 39, clone number: 645, (GenBank accession number: X95762)];

H. sapiens mRNA for UDP-GalNAc:polypeptide N-acetylgalactosaminyl transferase [SEQ ID NO: 40, clone number: 668, (GenBank accession number: X92689)];

Homo sapiens cDNA FLJ20463 fis, clone mRNA [SEQ ID NO: 41, clone number: 723, (GenBank accession number: AK000470)];

Novel gene similar to C. elegans hypothetical 55.2 KD protein F16A11.2, SW: P90838 mRNA [SEQ ID NO: 42, clone number: 731, (GenBank accession number: AL050255)];

Homo sapiens cDNA FLJ10986 fis, clone mRNA [SEQ ID NO: 43, clone number: 766, (GenBank accession number: AK001848)];

Human inhibitor of apoptosis protein 2 mRNA, complete cds [SEQ ID NO: 44, clone number: 778, (GenBank accession number: U45879)];

Human MB-1 mRNA, complete cds [SEQ ID NO: 45, clone number: 801, (GenBank accession number: M80462)];

Human tissue factor gene mRNA, complete cds. 1/1995 [SEQ ID NO: 46, clone number: 802, (GenBank accession number: J02846)];

Homo sapiens mRNA; cDNA DKFZp566E0224 (from clone DKFZp566E0224) [SEQ ID NO: 47, clone number: 817, (GenBank accession number: AL050031)];

Homo sapiens NADH-ubiquinone oxidoreductase subunit C1-B12 mRNA, complete cds [SEQ ID NO: 48, clone number: 818, (GenBank accession number: AF047183)];

Homo sapiens ASH1 mRNA, complete cds. 5/2000 [SEQ ID NO: 49, clone number: 839, (GenBank accession number: AF257305)];

Homo sapiens CGI-65 protein mRNA, complete cds [SEQ ID NO: 50, clone number: 845, (GenBank accession number: AF151823)];

Homo sapiens, clone 24451 mRNA sequence [SEQ ID NO: 51, clone number: 854, (GenBank accession number: AF070599)];

Homo sapiens ubiquitin hydrolyzing enzyme I (UBH1) mRNA, partial cds [SEQ ID NO: 52, clone number: 877, (GenBank accession number: AF022789)];

Homo sapiens mRNA for cysteine-rich protein [SEQ ID NO: 53, clone number: 882, (GenBank accession number: AJ006591)];

Human mRNA for KIAA0139 gene, complete cds [SEQ ID NO: 54, clone number: 883, (GenBank accession number: D50929)];

Homo sapiens clone 23819 white protein homolog mRNA, partial cds [SEQ ID NO: 55, clone number: 925, (GenBank accession number: AF038175)];

Homo sapiens connexin 26 (GJB2) mRNA, complete cds [SEQ ID NO: 56, clone number: 944, (GenBank accession number: M86849)];

Homo sapiens mRNA for KIAA0914 protein, complete cds [SEQ ID NO: 57, clone number: 1001, (GenBank accession number: AB020721)];

Homo sapiens mRNA for KIAA0907 protein, complete cds [SEQ ID NO: 58, clone number: 1008, (GenBank accession number: AB020714)];

H. sapiens mRNA for transcriptional intermediary factor [SEQ ID NO: 59, clone number: 1014, (GenBank accession number: 2X97674)];

Human cytosolic aspartate aminotransferase mRNA, complete cds [SEQ ID NO: 60, clone number: 1018, (GenBank accession number: M37400)];

Homo sapiens CGI-118 protein mRNA, complete cds [SEQ ID NO: 61, clone number: 1023, (GenBank accession number: AF151876)];

Human guanylate binding protein isoform II (GBP-2) mRNA, complete cds [SEQ ID NO: 62, clone number: 1028, (GenBank accession number: M55543)];

Homo sapiens cDNA FLJ10633 fis, clone mRNA [SEQ ID NO: 63, clone number: 1043, (GenBank accession number: AK001495)];

Homo sapiens chaperonin containing t-complex polypeptide 1, eta subunit (Ccth) mRNA, complete cds [SEQ ID NO: 64, clone number: 1046, (GenBank accession number: AF026292)]; and

Homo sapiens mRNA for Prer protein [SEQ ID NO: 65, clone number: 1060, (GenBank accession number: AJ005579)]

Each of the cancer-associated genes having the sequences shown in the above SEQ ID NOs: 1 to 65 can be roughly divided into genes each of which expression level is reduced due to canceration, and genes each of which expression level is increased due to canceration. The genes each of which expression level is reduced due to canceration include a gene consisting of a nucleic acid having at least one sequence selected from the group consisting of SEQ ID NOs: 2, 3, 4, 6, 8, 9, 11, 12, 17, 18, 21, 22, 23, 24, 26, 29, 30, 31, 36, 37, 39, 40, 41, 42, 43, 46, 47, 48, 49, 50, 51, 55, 57, 60 and 65. The genes each of which expression level is increased due to canceration include a gene consisting of a nucleic acid having at least one sequence selected from the group consisting of SEQ ID NOs: 1, 5, 7, 10, 13, 14, 15, 16, 19, 20, 25, 27, 28, 32, 33, 34, 35, 38, 44, 45, 52, 53, 54, 56, 58, 59, 61, 62, 63 and 64.

Furthermore, the cancer-associated genes shown by the present inventors also include 199 kinds of novel genes for which a gene having homology has not been found in the database. The nucleotide sequences of the genes are shown in SEQ ID NOs: 66 to 264. The genes are also encompassed in the present invention.

Each of the cancer-associated genes having the sequences shown in the above SEQ ID NOs: 66 to 264 can be roughly divided into genes each of which expression level is reduced due to canceration, and genes each of which expression level is increased due to canceration. The genes each of which expression level is reduced due to canceration include a gene consisting of a nucleic acid having at least one sequence selected from the group consisting of SEQ ID NOs: 66-81, 83-86, 88-104, 106-117, 119-123, 125-130, 132-156, 158-184, 186-189, 191-196, 198-201, 204-230, 232-237, 240, 241, 243, 245, 247-256, and 258-263. The genes each of which expression level is increased due to canceration include a gene consisting of a nucleic acid having at least one sequence selected from the group consisting of SEQ ID NOs: 82, 87, 105, 118, 124, 131, 157, 185, 190, 197, 202, 203, 231, 238, 239, 242, 244, 246, 257 and 264.

The cancer-associated gene used in the present invention includes a gene consisting of a nucleic acid capable of hybridizing to a nucleic acid having either a nucleotide sequence selected from the group consisting of SEQ ID NOs: 1 to 264 or a nucleotide sequence complementary to the sequence under stringent conditions, wherein an expression level of the gene in a cancer cell is altered as compared to that in a normal cell. The term “stringent conditions” referred to herein includes, for instance, those conditions described, for instance, in the above Molecular Cloning: A Laboratory Manual, 2nd Ed. and the like. Concretely, there are exemplified, for instance, conditions of incubating in a solution of 6×SSC (wherein 1×SSC means 0.15 M NaCl, 0.015 M sodium citrate, pH 7.0), containing 0.5% SDS, 0.1% bovine serum albumin (BSA), 0.1% polyvinylpyrrolidone, 0.1% Ficoll 400, 0.01% denatured salmon sperm DNA at 50° C. In addition, in the hybridization under the above stringent conditions, a nucleic acid having a sequence identity of at least about 80%, preferably 90% or more, more preferably 95% or more, to a nucleic acid having either a nucleotide sequence selected from the group consisting of SEQ ID NOs: 1 to 264 or a nucleotide sequence complementary to the sequence, can be obtained by carrying out the hybridization under the conditions of a lower ionic strength, for instance, conditions of 5×SSC, 4×SSC or the like and/or a higher temperature, for instance, a Tm value of the nucleic acid used of below 25° C., more preferably below 22° C., still more preferably below 20° C., or the like, concretely, although it may differ depending upon the Tm value of the nucleic acid used, 52° C. or higher, 55° C. or higher, 58° C. or higher, 60° C. or higher, 62° C. or higher, 65° C. or higher, or the like, from the viewpoint of further increasing the accuracy in the hybridization of the stringent conditions, carrying out more strict washing conditions, concretely using a buffer with a low ionic strength, for instance, 2×SSC, 1×SSC, 0.5×SSC, or the like, and carrying out washing at a higher temperature, for instance, at a Tm value of the nucleic acid used of below 40° C., more preferably below 30° C., still more preferably below 25° C., and concretely although it may differ depending upon the Tm value of the nucleic acid used, washing at 30° C. or higher, 37° C. or higher, 42° C. or higher, 45° C. or higher or the like. Here, Tm of the probe or primer can be calculated, for instance, by the following equation:

Tm=81.5−16.6(log₁₀[Na⁺])+0.41(% G+C)−(600/N).

wherein N is a strand length of the probe or primer; and % G+C is a content of guanine and cytosine residues in the probe or primer.

Here, as mentioned above, whether or not the gene consisting of a nucleic acid capable of hybridizing under the above conditions is a gene of which expression level is altered in a cancer cell as compared to that in a normal cell can be confirmed by comparing an expression level of a gene product in a normal cell and an expression level of a gene product in a cancer cell, for instance, by the Northern hybridization method, the RNase protection assay method, the hybridization method using DNA, or the like, thereby evaluating whether or not the expression level is altered between both cells mentioned above.

In addition, the cancer-associated gene used in the present invention is a gene consisting of a nucleic acid having a nucleotide sequence having a sequence identity of at least 80%, preferably 90% or more, and more preferably 95% or more, to either a nucleotide sequence selected from the group consisting of SEQ ID NOs: 1 to 264 or a nucleotide sequence complementary to the sequence, wherein an expression level of the gene in a cancer cell is altered as compared to that in a normal cell.

Here, as mentioned above, whether or not the gene consisting of a nucleic acid having a nucleotide sequence having the above sequence identity is a gene of which expression level is altered in a cancer cell as compared to that in a normal cell can be confirmed by comparing an expression level of a gene product in a normal cell and an expression level of a gene product in a cancer cell, for instance, by the Northern hybridization method, the RNase protection assay method, the hybridization method using DNA, or the like, thereby evaluating whether or not the expression level is altered between both cells mentioned above.

The term “sequence identity” as used herein refers to sequence similarity between two nucleic acids. The above “sequence identity” can be determined by comparing the two sequences aligned in an optimal state over the region of sequence to be compared. Here, the nucleic acid to be compared may have addition or deletion (for instance, gap and the like) as compared to a reference sequence (for instance, consensus sequence and the like) for an optimal alignment of the two sequences.

The numerical value (percentage) of the sequence identity can be calculated by determining the identical nucleic acid bases existing in both the sequences, determining the number of matched sites, subsequently dividing the number of the above matched sites by the total number of bases within the region of sequence to be compared, and multiplying the obtained quotient by 100. The algorithms for obtaining the optimal alignment and homology include, for instance, the local homology algorithm of Smith et al. [Add. APL. Math., 2, 482 (1981)], the homology alignment algorithm of Needleman et al. [J. Mol. Biol., 48, 443 (1970)], and the homology search method of Pearson et al. [Proc. Natl. Acad. Sci. USA, 85, 2444 (1988)]; more concretely, there are included the dynamic programming method, the gap penalty method, the Smith-Waterman algorithm, the Good-Kanehisa algorithm, the BLAST algorithm, the FASTA algorithm and the like.

The sequence identity for the nucleic acids is, for instance, determined by using sequence analysis software, concretely BLASTN or the like. The above BLASTN is generally available on the homepage address http://www/ncbi.nlm.nih.gov/BLAST.

Here, among the above SEQ ID NOs: 1 to 264, a nucleic acid comprising a sequence for a cancer-associated gene selected from the group consisting of:

(a′) a nucleic acid having either a nucleotide sequence selected from the group consisting of SEQ ID NOs: 66 to 264 or a nucleotide sequence complementary to the sequence, wherein the nucleic acid comprises the sequence of a gene of which expression level in a cancer cell is altered as compared to that in a normal cell;

(b′) a nucleic acid capable of hybridizing to a nucleic acid having either a nucleotide sequence selected from the group consisting of SEQ ID NOs: 66 to 264 or a nucleotide sequence complementary to the sequence under stringent conditions, wherein the nucleic acid comprises the sequence of a gene of which expression level in a cancer cell is altered as compared to that in a normal cell; and

(c′) a nucleic acid having a nucleotide sequence having at least 80% sequence identity to either a nucleotide sequence selected from the group consisting of SEQ ID NOs: 66 to 264 or a nucleotide sequence complementary to the sequence, wherein the nucleic acid comprises the sequence of a gene of which expression level in a cancer cell is altered as compared to that in a normal cell,

which are 199 kinds of novel genes for which a gene having homology has not been found in the database,

is also encompassed in the present invention.

In the detection method of the present invention, since an alteration of the expression of the above gene is used as an index, there is exhibited an excellent that a sample from an individual suffering from a cancer, concretely a cancer in a digestive organ such as gastric cancer, esophageal cancer or colorectal cancer, more concretely papillary adenocarcinoma, well differentiated adenocarcinoma, moderately differentiated adenocarcinoma, poorly differentiated adenocarcinoma, signet-ring cell carcinoma, mucinous carcinoma, or the like, for instance, a cancer cell can be detected at an early stage.

Furthermore, in the detection method of the present invention, since the alteration in expression of the above gene is used as an index, there can be detected at surprisingly high accuracy a cancer, concretely a cancer in the digestive organ such as gastric cancer, esophageal cancer or colorectal cancer, more concretely papillary adenocarcinoma, well differentiated adenocarcinoma, moderately differentiated adenocarcinoma, poorly differentiated adenocarcinoma, signet-ring cell carcinoma or mucinous carcinoma. Also, there can be detected at a surprisingly high accuracy a sample from an individual suffering from a cancer, concretely a cancer in the digestive organ such as gastric cancer, esophageal cancer or colorectal cancer, more concretely papillary adenocarcinoma, well differentiated adenocarcinoma, moderately differentiated adenocarcinoma, poorly differentiated adenocarcinoma, signet-ring cell carcinoma, mucinous carcinoma, or the like, for instance, a cancer cell.

According to the detection method of the present invention, the detection of a cancer can be carried out by comparison of expression levels of the above genes. In the present invention, the “gene of which expression level is altered in a cancer cell as compared to that in a normal cell” used as an index, i.e., a cancer-associated gene, may be used singly as an index, or may desirably be used in combination of several kinds of genes. The combinations are not particularly limited.

In the present invention, the judgment of whether or not an individual who is a source for a sample to be tested is suffering from a cancer, or the judgment of whether or not a sample to be tested, for instance, a cell, is a sample associated with a cancer, for instance, a cancer cell, can be carried out by first confirming a normal value range for an expression level of a cancer-associated gene used as an index of canceration by an appropriate detection method using a plurality of normal tissues from an individual who is a source for a sample to be tested, thereafter measuring an expression level of the cancer-associated gene in a sample to be tested, concretely in a sample excised from the body, and comparing the normal value range and the value for the expression level of the cancer-associated gene in the sample to be tested. In the present specification, the above individual who is a source for a sample to be tested is also referred to as “specimen.”

In other words, in a case where a cancer-associated gene used as an index is a gene of which expression level is decreased due to canceration, a sample to be tested can be judged cancer-positive, if the expression of the cancer-associated gene is not confirmed in the sample to be tested, concretely a sample excised from the body, or if the expression level of the cancer-associated gene is lower than that of the normal value range. On the other hand, in a case where a cancer-associated gene used as an index is a gene of which expression level is increased due to canceration, the sample to be tested can be judged cancer-positive, if the expression level of the cancer-associated gene is higher than that of the normal value range.

In addition, in a case where a judgment is made with a combination of several kinds of cancer-associated genes as indices, the alterations in the expression of the several kinds of cancer-associated genes used as indices of canceration are evaluated by using an appropriate detection method using a normal tissue, and a false positive ratio is calculated. Next, the judgment is carried out by measuring the alteration in the expression of the cancer-associated genes in a sample excised from the body, and carrying out the comparison with the false positive ratio.

Here, the false positive ratio is calculated as described below.

The expression of a plurality of genes in a normal gastric tissue sample from a gastric cancer patient is analyzed against a gastric tissue collected from a normal individual as a control sample. As the above control sample, there may be usually used tissues from a plurality of normal individuals in admixture.

In this analysis, even in a normal tissue, there is observed a significant alteration in expression in some of the genes compared to that of the control sample. The analysis of gene expression is carried out for normal gastric tissue samples from a plurality of gastric cancer patients, and an average of the number of genes each of which expression is significantly altered is calculated together with its standard deviation (SD). The thus-obtained value:

[Average of the number of genes each of which expression is significantly altered +2SD]

is defined as the false positive ratio.

In the sample to be tested, in a case where alteration in expression is observed in the number of genes exceeding the above false positive ratio, the sample can be judged to be cancer-positive.

The alteration in expression of a cancer-associated gene may be either an alteration in the expression level of mRNA of the gene or an alteration in the expression level of the protein encoded by the gene.

In the case where an alteration in expression of a cancer-associated gene is evaluated by an alteration in the expression level of mRNA of the gene, the amount of mRNA can be determined by the hybridization method or the nucleic acid amplification method.

The above nucleic acid amplification method includes polymerase chain reaction, strand displacement reaction and the like.

Also, the hybridization method includes the Northern hybridization method, the dot blot hybridization method, and the hybridization method using a DNA array or the like.

The hybridization method using a DNA array and the RT-PCR method are preferred, from the viewpoint of detection sensitivity of mRNA. The hybridization method using a DNA array is especially preferred, from the viewpoint of simultaneously determining expressions of a large number of genes.

The above RT-PCR method refers to a method comprising synthesizing cDNA by a reverse transcription reaction with mRNA as a template, and thereafter carrying out nucleic acid amplification by PCR. In the present specification, the above method for nucleic acid amplification includes the PCR (Polymerase Chain Reaction) method (U.S. Pat. No. 4,683,202); the ICAN (Isothermal and Chimeric primer-initiated Amplification of Nucleic Acid) method; the Strand Displacement Amplification (SDA) method [Walker G. T. et al., Nucleic Acids Res., 20, 1691-1696 (1992)]; the Nucleic Acid Sequence-Based Amplification (NASBA) method [Compton, J., Nature, 350, 91-92 (1991)]; and the like. The reaction conditions for the nucleic acid amplification are not particularly limited.

The region to be amplified in the cDNA of the gene used as an index in the measurement of mRNA may be a full-length cDNA, or may be a partial region of the cDNA, as long as the amplified product can be identified.

It is desirable that the primer pair usable in the above nucleic acid amplification reaction are designed so as to specifically amplify cDNA. In the confirmation of the amplified product, when the amplified product is confirmed by, for instance, subjecting an amplified DNA fragment to agarose gel electrophoresis and then staining the gel with ethidium bromide (EtBr), there are some cases where a large number of amplified DNA fragments having the number of bases of the same level are produced by the nucleic acid amplification reaction, thereby resulting in incomplete separation of each amplified DNA fragment so that the existing amount of the amplified DNA fragments corresponding to the mRNA of the cancer-associated gene to be detected cannot be confirmed. In such cases, cDNA other than the cDNA to be detected may be amplified, if the amplified product of the region to be amplified in the cDNA of the gene used as an index can be confirmed.

The amount of amplified DNA can be evaluated, for instance, by mobility of the detected band and its signal intensity which is obtained by subjecting a solution containing the product obtained by the above nucleic acid amplification reaction to agarose gel electrophoresis, using a labeled probe specifically hybridizing to the desired amplified fragment. Therefore, there can be judged that the more intensive the above signal intensity obtained by using a given amount of a crude RNA extracted from a sample excised from the body, the higher the expression level of the mRNA. The label for the above probe includes, for instance, but is not particularly limited to, radioactive substances represented by ³²P, as well as fluorescent substances represented by fluorescein.

In a case where an amplified product is obtained in a sufficient amount, the amount can be confirmed by the mobility and fluorescence intensity of the amplified DNA fragment which is obtained by carrying out agarose gel electrophoresis, and thereafter staining the gel with EtBr. Therefore, there can be judged that the higher the fluorescence intensity, the higher the expression level of the cancer-associated gene.

In order to carry out a more accurate judgment, a degree of amplification may be quantified and numerically expressed. For instance, the degree of amplification can be quantified by carrying out nucleic acid amplification by the quantitative PCR method (Japanese Unexamined Patent Publication No. HEI-5-504886) or the TaqMan method [Linda G. Lee et al., Nucleic Acids Res., 21, 3761-3766 (1993)], during the stage of the nucleic acid amplification reaction.

In the hybridization method using a DNA array, the DNA array used may be an array comprising the nucleic acids each corresponding to each of the genes of which expression levels are to be determined or fragments thereof, wherein each of the nucleic acids or fragments thereof is immobilized to a given region on a support, and the number of genes and combination thereof are not particularly limited. The above DNA array is exemplified by, for instance, the DNA array of the present invention described below.

The determination of the expression level of the genes using a DNA array is, for instance, carried out by the following procedures.

Concretely, a labeled nucleic acid such as labeled mRNA obtained by labeling mRNA in the sample or cDNA labeled with mRNA as a template is prepared, and thereafter hybridization is carried out between the labeled nucleic acid and the DNA array obtained, whereby the mRNAs expressed in the sample can be simultaneously detected, and further the expression levels thereof can be determined. Labeling substances used for labeling include, for instance, radioisotopes, fluorescent substances, chemical substances, and substances having a chromophore, more concretely fluorescent substances such as Cy2, Fluor X, Cy3, Cy5, Cy7, fluorescein isothiocyanate (FITC), Texas Red, and rhodamine. Also, since simultaneous detection can be carried out, it is desirable that each of the sample to be tested and the sample used as control is labeled using a different fluorescent substance with each of two or more kinds of fluorescent substances. The detection method for label can be appropriately selected according to the labeling substance used. For instance, when the above Cy3 and Cy5 are used as labeling substances, Cy3 can be detected by scanning at a wavelength of 532 nm and Cy5 at a wavelength of 635 nm. Here, the intensity of the label is used as an index of the expression level of the gene. As the hybridization conditions, those suitable for the DNA array and the labeled cDNA used may be appropriately selected.

In order to determine the difference in gene expression between the sample to be tested and the control sample, errors adjusted by measurement means or the like may be corrected using a nucleic acid from a non-lesion portion; a nucleic acid corresponding to a housekeeping gene [for instance, glycelaldehyde 3-phosphate dehydrogenase gene, cyclophilin gene, β-actin gene, α-tubulin gene, phospholipase A2 gene, and the like]; and the like. Also, as a negative control for confirming that the hybridization is not nonspecific, there can be, for instance, used a nucleic acid from a different organism, including, for instance, plasmid pUC18.

In addition, alterations in the expression level of the cancer-associated gene can be confirmed by using expression of a protein as an index. The expression of the above protein may be confirmed by measuring the biological activity of the protein, or alternatively, the protein may be detected using an antibody against the protein, from the viewpoints of simplicity and easiness in handling. The antibody is also encompassed in the present invention.

The antibody used in the detection method of the present invention is an antibody capable of specifically binding to a protein encoded by the cancer-associated gene. The antibody may be a fragment of the antibody, as long as the fragment is capable of specifically binding to a protein encoded by the cancer-associated gene (hereinafter also referred to as cancer-associated protein).

Therefore, in the detection method using an antibody, a defined amount of a crude protein extracted from a sample to be tested, concretely a sample excised from the body, and the above antibody are mixed, and it can be judged that the expression level of the cancer-associated gene is high if the amount of the antibody bound is large.

The cancer-associated protein as an antigen for obtaining the above antibody may be obtained by purification from a cancer cell expressing the above cancer-associated gene, or obtained by using a genetic engineering technique.

For instance, a full-length nucleic acid encoding a cancer-associated protein can be obtained by a combination of the above DD method and screening of a cDNA library prepared from a cell expressing the desired protein, or the like. In addition, in a case where the cancer-associated gene is not a nucleic acid corresponding to a full-length cDNA, cDNA containing a full-length open reading frame can also be obtained by determining its 5′-terminal and/or 3′-terminal using the genes by the 5′-RACE method or a method comprising deducing a translational region corresponding to already known nucleotide sequences of genomic DNA or other means, and carrying out a commonly used method such as PCR.

The above cancer-associated protein used in the preparation of the antibody can be obtained by incorporating the cDNA obtained into an appropriate expression vector, and expressing it in an appropriate host. Further, the above protein may be expressed as a fusion protein. For example, the cDNA obtained may be incorporated into a vector including, for instance, glutathione S-transferase (GST) fusion protein vector (pGEX4T or the like); a vector carrying a tag (His tag, or the like) sequence, to express the fusion protein by using the resulting recombinant vector, or alternatively, an appropriate peptide chain may be added to an N-terminal or a C-terminal derived from other proteins and then allowed to be expressed in order to increase the expression level of a desired protein. In the case where the above protein is expressed as a fusion protein, the purification of the desired protein can be facilitated by using a carrier having an affinity with the peptide chain added.

In other words, the above antibody can be obtained, for instance, by the steps of obtaining a full length of the above cancer-associated gene; incorporating the resulting full-length gene into an appropriate vector (commonly used plasmid vector, phage vector or viral vector) by means of commonly used gene recombination techniques; introducing the resulting recombinant vector to a host (Escherichia coli cell such as HB101 strain, C600 strain, JM109 strain or DH5α strain; an yeast cell such as Saccharomyces cerevisiae; an animal cell such as COS cell, CHO cell, L cell, 3T3 cell, Vero cell, and HeLa cell; insect cell such as sf9; or the like) appropriate for the production of the polypeptide encoded by the gene; culturing the resulting transformant under conditions appropriate for the expression of the polypeptide to give a polypeptide; purifying the polypeptide and immunizing an appropriate animal (for instance, a rabbit or the like) using the resulting polypeptide, to give an antibody. The above vector includes plasmid vectors such as vectors, pUC18, pUC19, pBR322, pCR3 and pCMVSPORT; phage vectors such as λZAPII and λgt11, in a case where the host is Escherichia coli; pYES2, and the like in the case where the host is an yeast; pAcSGH is NT-A and the like in a case where the host is an insect; and pKCR, cDM8, and the like in a case where the host is an animal cell. Incidentally, in a case where an expressed polypeptide is obtained as an insoluble inclusion body, a soluble polypeptide can be obtained in accordance with a commonly used method for producing a solubilized protein.

In addition, the antigen for obtaining an antibody is the above cancer-associated protein, or a region which can exist in the protein, wherein the antigen may be either a peptide having an antigenic determinant on which the antibody or a peptide having an amino acid sequence region specific to the protein can be specifically bound.

The antibody or a fragment thereof used in the present invention is the above cancer-associated protein or a region which can exist in the protein, which may be those having an ability of specifically binding to a peptide having an antigenic determinant on which the antibody or a peptide having an amino acid sequence region specific to the protein can be specifically bound. The antibody may be any of polyclonal antibodies and monoclonal antibodies. Further, antibodies modified by known techniques or antibody derivatives, for instance, humanized antibodies, Fab fragments, single-chain antibodies, and the like, can also be used.

Here, as the method for obtaining the above antibody, a peptide is immunized with an animal together with an adjuvant by a conventional method, thereby obtaining as antiserum. Also, the antibody can be obtained as a monoclonal antibody according to the method of Galfre, G. et al. [Nature, 266, 550-552 (1977)]. Furthermore, the fragment of the antibody is obtained by purifying the resulting antibody and thereafter treating the product with a peptidase or the like.

In addition, the antibody or a fragment thereof of the present invention may be subjected to various modifications in order to facilitate the detection by enzyme immunoassay, fluoroimmunoassay, luminescent immunoassay, or the like.

The method for detecting a protein using the antibody includes, for instance, enzyme immunoassay, fluoroimmunoassay, luminescent immunoassay, and the like, more concretely, for instance, the Western blot method, the ELISA and the like.

The above Western blot method is a method for detecting a protein transferred to a nitrocellulose membrane or the like with an antibody. Concretely, in the above Western blot method, a cell is treated with a surfactant to extract intracellular proteins, the extract obtained is separated by SDS-polyacrylamide gel electrophoresis, the gel after the separation is transferred onto a nitrocellulose membrane or the like, and the transfer membrane obtained and an antibody specific to a protein to be detected are reacted. The antibody bound to a protein can, for instance, be secondarily detected using a ¹²⁵I-labeled protein A, a peroxidase-bound anti-IgG antibody, or the like.

One of the features of the DNA array of the present invention resides in that each of at least two kinds of genes selected from the group consisting of the above (a) to (c) or fragments thereof are immobilized to a given region on a support. The array of the present invention is suitable for the method for detecting a cancer of the present invention.

The array of the present invention is useful in the detection of a cancer or cancer cells because at least two kinds of genes selected from the group consisting of the above (a) to (c) or fragments thereof are immobilized thereto. Also, by simultaneously immobilizing known genes or fragments thereof, which have been known to be associated with canceration of cell or progression of cancer, the cancer can be detected at an early stage at higher accuracy, and at the same time progression levels of cancer and a grade of malignancy can be evaluated.

The term “each of . . . immobilized to a given position” as used herein means that a position at which a nucleic acid corresponding to each gene or a fragment thereof is immobilized has been previously determined on the support. In other words, when the array as described above is used, there can be readily known from the position of the detected signal which of the genes the nucleic acid or fragment this signal is originated.

The DNA array of the present invention includes an array comprising at least two kinds of genes selected from the group consisting of the above (a) to (c) or fragments thereof, wherein each of at least two kinds of genes are immobilized to a support represented by slide glass or the like.

The support used in the array of the present invention may be of any material which can be used for hybridization, and usually includes slide glass, silicone chips, nitrocellulose membranes, nylon membranes, and the like. More preferably, the support is made of a non-porous material having a structure with a smooth surface. For instance, glass such as slide glass can be preferably used.

The surface of the support may be any of those to which single-stranded DNA can be immobilized by a covalent bond or a non-covalent bond. From this viewpoint, a support with a surface having a hydrophilic or hydrophobic functional group can be preferably used. The functional group on the surface of the support includes, for instance, hydroxyl group, amino group, thiol group, aldehyde group, carboxyl group, an acyl group and the like. These functional groups may exist as the surface properties of the support itself, or may be introduced by surface treatment.

The above surface-treated material includes a product obtained by treating glass with a commercially available silane-coupling agent such as an aminoalkylsilane, a product obtained by treating glass with a polycation such as polylysine or polyethyleneimine, and the like. In addition, a part of these treated slide glasses are commercially available.

In the array of the present invention, the nucleic acid or a fragment thereof may be single-stranded or double-stranded. For instance, the array of the present invention may be a DNA array in which the above nucleic acid or a fragment thereof is arranged and immobilized to a support as a denatured double strand, or a DNA array in which at least a portion of the immobilized DNA is a single-stranded DNA. Also, the array of the present invention may be a DNA array in which double-stranded DNA is arranged and spotted on the same support under denaturing conditions.

Furthermore, in the array of the present invention, the density of the immobilized nucleic acid or fragment thereof on the support can be set appropriately according to the purpose of use and the number of the nucleic acids or fragments thereof to be immobilized. For instance, when the amount of a sample for detection is very small, a high-density array can be used: For instance, an array in which a nucleic acid, especially DNA, is immobilized at a density of 30 dots/cm² or more, can be preferably used.

The nucleic acid or fragment thereof immobilized on the support may be either a polynucleotide or an oligonucleotide. In addition, its production method is not particularly limited, and those chemically synthesized, those isolated and purified from a naturally-occurring nucleic acid, or those enzymatically synthesized, and further those combining these can be used.

It is desired that the nucleic acid or fragment thereof immobilized on the support is, for instance, but not particularly limited to, a sequence which is a double-stranded polynucleotide having a strand length of 50 bases in length or more or a derivative thereof, not containing a repeat sequence such as polyA sequence or Alu sequence, and having high specificity to the desired gene. For instance, there can be preferably used a single-stranded DNA or a derivative thereof obtained by denaturing a double-stranded DNA prepared by enzymatic amplification by PCR (polymerase chain reaction) or the like, wherein the double-stranded DNA is a double-stranded polynucleotide having a strand length of 50 bases in length or more or a derivative thereof upon immobilization to the support of the DNA.

The above derivative includes those subjected to modification so that its immobilization to the surface of the support can be carried out. The derivative includes, for instance, DNA in which a functional group such as amino group or thiol group is introduced at a 5′-terminal thereof.

Also, there can be used a DNA prepared, for instance, by amplification by PCR or the like with a genomic DNA library or a cDNA library as a template.

The array can be prepared by, for instance, immobilizing the nucleic acids each corresponding to each of the above genes or fragments thereof to a support into which amino group is introduced by a known method. Also, the array of the present invention in which the nucleic acids each corresponding to each of the genes are arranged and immobilized can be prepared by carrying out the above immobilization procedure by using an apparatus for preparing a DNA array, for instance, an apparatus for preparing DNA chip manufactured by Affymetrix.

It is desired that the strand length of the fragment of the nucleic acid is, for instance, but not particularly limited to, about 100 bases in length to about 1000 bases in length. Also, the strand length that is shorter or longer than the above strand length may be used, as long as the fragment can be specifically hybridized in the hybridization with a nucleic acid from the sample to be tested.

There can be also provided a method for providing evaluation information by analyzing the results obtained by the above detection method by an analytical means (for instance, image processing by a computer or the like), recording or displaying the analytical results on a recording medium such as paper, a computer-readable recording medium or the like. The method for providing evaluation information is also encompassed in the present invention.

The above method for providing evaluation information, for instance, comprises the steps of:

(i) carrying out the above detection method to examine an expression level or expression profile of a cancer-associated gene in a sample to be tested;

(ii) inputting data for the expression level or expression profile obtained in the above step (i) into a computer;

(iii) comparing the data for the sample to be tested inputted into the above step (ii) with those of the control sample, thereby distinguishing the inputted data by a program for sorting those showing a difference between the sample to be tested and the control sample; and

(iv) providing a means for displaying the possibility that a sample to be tested distinguished in the above step (iii) is cancer or contains a cancer cell.

The detection method of the present invention is a method which is carried out by evaluating expression of a cancer-associated gene selected from the group consisting of the above (a) to (c), as described above, wherein mRNA or protein expressed from the above cancer-associated gene is detected as an index. Therefore, a kit for detecting a cancer comprising a reagent by which the above mRNA or protein can be detected can be utilized in the method for detecting a cancer of the present invention. The kit for detecting a cancer is also encompassed in the present invention. According to the kit, a sample associated with a cancer, for instance, a cancer cell or the like, can also be detected.

The kit for detecting a cancer includes a kit comprising at least one kind of an oligonucleotide consisting of a nucleic acid capable of hybridizing to mRNA transcribed from a gene selected from the group consisting of the above (a) to (c) under stringent conditions (kit for detecting mRNA); and a kit comprising an antibody or a fragment thereof, capable of binding to a protein expressed from the gene selected from the group consisting of the above (a) to (c) (kit for detecting a protein).

The kit for detecting a cancer (kit for detecting mRNA) of the present invention is a kit for measuring the amount of mRNA transcribed from the gene selected from the group consisting of the above (a) to (c). The kit includes \mathring{1} a kit comprising at least one kind of a probe capable of hybridizing to the above mRNA; and \mathring{2} a kit comprising at least one pair of a primer pair for amplifying the above mRNA.

Also, the kit for detecting a cancer (kit for detecting a polypeptide) of the present invention includes \mathring{3} a kit for detecting a cancer-associated protein translated from the above mRNA or a polypeptide originated therefrom with an antibody or a fragment thereof, capable of specifically binding to the cancer-associated protein or a polypeptide originated therefrom.

Since the kit of the present invention comprises a probe and/or primers for detecting mRNA or a fragment thereof, expressed from a gene of which expression level is altered specifically in cancer, or an antibody or a fragment thereof against a protein encoded by a gene of which expression level is altered specifically in cancer or a polypeptide originated therefrom, the kit can be used in the above method for detecting a cancer, and allows to carry out simpler, easier and quicker procedures.

The kit for detecting a cancer (kit for detecting mRNA) of the present invention [the above kit \mathring{1} or \mathring{2}] comprises primers and/or a probe for detecting mRNA or a fragment thereof, expressed from at least one kind of gene of which expression is altered in a cancer, especially in a gastric cancer, by a nucleic acid amplification method, for instance, the RT-PCR method.

The above primers and/or probe include 1) a nucleic acid of a gene selected from the group consisting of the above (a) to (c), namely at least one kind of a nucleic acid selected from the group consisting of:

(A) a nucleic acid having either a nucleotide sequence selected from the group consisting of SEQ ID NOs: 1 to 264 or a nucleotide sequence complementary to the sequence, wherein the nucleic acid comprises the sequence of a gene of which expression level in a cancer cell is altered as compared to that in a normal cell;

(B) a nucleic acid capable of hybridizing to a nucleic acid having either a nucleotide sequence selected from the group consisting of SEQ ID NOs: 1 to 264 or a nucleotide sequence complementary to the sequence under stringent conditions, wherein the nucleic acid comprises the sequence of a gene of which expression level in a cancer cell is altered as compared to that in a normal cell; and

(C) a nucleic acid having a nucleotide sequence having at least 80% sequence identity to either a nucleotide sequence selected from the group consisting of SEQ ID NOs: 1 to 264 or a nucleotide sequence complementary to the sequence, wherein the nucleic acid comprises the sequence of a gene of which expression level in a cancer cell is altered as compared to that in a normal cell, or a fragment thereof; 2) a nucleic acid having a nucleotide sequence complementary to the nucleic acid or a fragment thereof; or 3) a nucleic acid capable of hybridizing to a nucleic acid having a nucleotide sequence complementary to the nucleic acid or a fragment thereof under stringent conditions.

The above term “stringent conditions” is not particularly limited, and includes, for instance, conditions in which the probe or primers are incubated overnight in a solution of 6×SSC, 0.5% SDS, 5× Denhardt's, 100 μg/ml denatured herring sperm DNA at a temperature of [Tm −25° C. of the above-mentioned primers and/or probe]. As to the temperature conditions, the stringency can be increased by more approximating to a Tm value (for instance, Tm −24° C., Tm −22° C., Tm −20° C., and the like). In addition, the stringency can be increased by carrying out hybridization under the conditions of, for instance, 0.5×SSC, 4×SSC, and the like.

In addition, as the primers and/or a probe, a nucleic acid having a sequence identity of at least about 80%, more preferably 90% or more, still more preferably 95% or more to the nucleotide sequence selected from the group consisting of SEQ ID NOs: 1 to 264, or a nucleic acid having any of the nucleotide sequences complementary to the sequence, can be obtained by carrying out the hybridization under stricter washing conditions, concretely under the conditions of a lower ionic strength, for instance, 2×SSC, 1×SSC, 0.5×SSC, or the like, and carrying out washing at a higher temperature, for instance, at a Tm value of the nucleic acid used of below 40° C., more preferably below 30° C., still more preferably below 25° C., and concretely although it may differ depending upon the Tm value of the nucleic acid used, washing at 37° C. or higher, 42° C. or higher, 45° C. or higher or the like.

The nucleotide sequence of the above primers may be a sequence capable of specifically amplifying a nucleic acid corresponding to the above gene under the reaction conditions for an ordinary nucleic acid amplification method, especially RT-PCR.

On the other hand, the nucleotide sequence of the probe may be a sequence of a nucleic acid capable of hybridizing to a nucleic acid corresponding to the above gene under the above stringent conditions.

Here, Tm of the probe or primers can be calculated, for instance, by the following equation:

Tm=81.5−16.6(log₁₀[Na⁺])+0.41(% G+C)−(600/N),

wherein N is a strand length of the probe or primer; and % G+C is a content of guanine and cytosine residues in the probe or primer.

In addition, when the strand length of the probe or primer is shorter than 18 bases, Tm can be deduced from a sum of a product of the contents of A+T (adenine+thymine) residues multiplied by 2° C., with a sum of a product of the contents of G+C residues multiplied by 4° C. [(A+T)×2+(G+C)×4].

The strand length of the above probe is not particularly limited. It is preferable that the strand length is 15 bases in length or more, more preferably 18 bases in length or more, from the viewpoint of preventing nonspecific hybridization. For instance, a probe having a strand length of 15 continuous bases in length or more in the nucleotide sequence selected from the group consisting of SEQ ID NOs: 1 to 264 of Sequence Listing or a sequence complementary to the nucleotide sequence can be used.

The strand length of the primers is not particularly limited. It is desired that the primer has a strand length, for instance, of 15 to 40 bases in length, preferably 17 to 30 bases in length. For instance, there can be used primers each having a nucleotide sequence of continuous 15 to 40 bases in length in the nucleotide sequence selected from the group consisting of SEQ ID NOs: 1 to 264 of Sequence Listing or a sequence complementary to the sequence.

A sequence suitable for the above probe and primers can be obtained by using a commercially available software or the like by which the secondary structure formation can be predicted on the bases of, for instance, the above Tm value and the like. The software includes OLIGO Primer Analysis Software (manufactured by Takara Shuzo Co., Ltd.) and the like. One of ordinary skill in the art can design a sequence which cannot detect a known gene not specific to a cancer using the above software. The sequence which cannot detect a known gene not specific to a cancer includes, for instance, a sequence having sequence identity of 30% or less, preferably 20% or less, more preferably 10% or less, still more preferably 5% or less, especially preferably 0% throughout the known gene not specific to a cancer. As the sequence which cannot detect “a known gene not specific to a cancer” having entirely or partially high homology to nucleotide sequences selected from the group consisting of SEQ ID NOs: 1 to 264 of Sequence Listing used in the present invention, a sequence suitable for the probe or primer can be selected from a particular portion having low homology or a portion characteristic to a nucleotide sequence selected from the group consisting of SEQ ID NOs: 1 to 264.

In addition, the above probe and primers may have various labels appropriate for the detection of the probe or primers, for instance, fluorescent labels, radioactive substances labels and the like.

Concrete examples of the kit for detecting a cancer of the present invention (the kit for detecting mRNA) includes a kit comprising oligonucleotides suitable as a probe for hybridization; and a kit comprising at least one pair of oligonucleotides, each being 15 to 40 bases in length, suitable as primers.

In the kit for detecting a cancer (the kit for detecting a polypeptide) of the present invention [the kit of the above (3)], the antibody is not particularly limited, as long as the antibody has an ability of specifically binding to the above cancer-associated protein or a polypeptide originated therefrom. The antibody may be any of polyclonal antibodies and monoclonal antibodies. Further, antibodies modified by known techniques or antibody derivatives, for instance, humanized antibodies, Fab fragments, single-chain antibodies, and the like, can also be used. The above antibody of the present invention can be readily prepared by immunizing a rabbit, a rat, a mouse or the like using all or a part of the above polypeptide of the present invention in accordance with the method described in, for instance, Current Protocols in Immunology, edited by John E. Coligan, published by John Wiely & Sons, Inc., 1992. The resulting antibody is purified and treated with a peptidase or the like to give a fragment of an antibody. Also, an antibody can be prepared by genetic engineering means. Further, the antibody or a fragment thereof of the present invention may be subjected to various modifications in order that the detection by enzyme immunoassay, fluoroimmunoassay, luminescent immunoassay, or the like is facilitated.

Also, the antibody or a fragment thereof includes those capable of specifically binding to a partial fragment of the polypeptide.

The kit of the present invention may further comprise a reagent suitable for the detection of various labels or various modifications.

According to the present invention, there is also provided a method for suppressing proliferation of a cancer cell using a specific binding substance to the above cancer-associated genes (a) to (c), or a cancer-associated protein or a polypeptide originated therefrom. The method for suppressing proliferation of a cancer cell is also encompassed in the present invention.

The above specific binding substance includes nucleic acids, antibodies, cytotoxic T lymphocytes (CTL) and the like.

For instance, the bcr-abl chimeric protein, which is often detected in chronic myelocytic leukemia, possesses high tyrosine kinase activity and plays a key role in the genesis and proliferation of leukemia. An antisense oligonucleotide for a gene encoding this chimeric protein is capable of suppressing the proliferation of these gene-expressed tumors in vivo (Skorski, T., Proc. Natl. Acad. Sci., USA 91 4504 1994). On the other hand, there has been known that a cancer-specific peptide of a protein specifically expressed in a cancer cell is targeted for T cell immune responses to a cancer cell, and T cell that is reactive to the fusion protein is obtained by immunization with a peptide in the vicinity of the fused portion of this fusion protein (Chen, W., Proc. Nat. Acad. Sci. USA 89, 1468 1992). As the operational procedures, there can be utilized, for instance, the techniques described in the following reports. In other words, CD4+ T cell reacting specifically to ras peptide in which 12th amino acid glycine is substituted with another amino acid in human T cell, and having HLA-DR restriction is separated (Jung, S., J. Exp. Med. 173, 273 1991), and cytotoxic T lymphocytes against a peptide consisting of eight amino acids including its mutated site can be induced from a mouse immunized with a recombinant vaccinia virus capable of producing the ras peptide having a mutation in 61st amino acid (Skipper, J., J. Exp. Med. 177, 1493 1993). Furthermore, the in vivo proliferation of a cancer cell having the same mutation is suppressed in a mouse immunized with a soluble mutated ras protein prepared by genetic recombination technique (Fenton, R. G., J. Natl. Cancer Inst., 85, 1294 1993), and CTL exhibiting cytotoxic activity on a cancer cell expressing the same mutated ras is obtained from splenocyte sensitized with the mutated ras peptide (Peace, D. J., J. Exp. Med., 179, 473, 1994).

Concretely, one of significant features of the method of the present invention for suppressing proliferation of a cancer cell resides in that the proliferation of a cancer cell is suppressed by using a specific binding substance that is capable of specifically binding to:

(1) at least one kind of gene selected from the group consisting of the above (A) to (C), or

(2) the polypeptide encoded by the above (1).

The specific binding substance capable of specifically binding to the above (1) includes, for instance, antisense oligonucleotides, anti-DNA antibodies and the like. Also, there can be utilized a control by RNAi [RNA interference: Nature, 391, 806-811 (1998)] using double-stranded RNA.

The specific binding substance capable of specifically binding to the above (2) includes, for instance, cytotoxic T lymphocytes against the polypeptide (2), and an antibody, a compound, a protein, or the like capable of specifically binding to the polypeptide (2).

Therefore, there is a possibility that the cell proliferation can be controlled by using a similar antisense oligonucleotide for a gene of which association with canceration of a cell is found in the present invention. Furthermore, when a T cell reactive to the polypeptide (cancer-associated protein or the like) encoded by a gene of which expression level is considered to be increased due to canceration is obtained, the proliferation of a cell expressing the protein at a high level can be suppressed.

The above antisense oligonucleotide has, but not particularly limited, a sequence complementary to the mRNA transcribed from a target gene. For instance, an antisense oligonucleotide of 20 bases in length or more can be prepared by chemical synthesis or enzymatic synthesis. The term “antisense oligonucleotide” as used herein means an oligonucleotide having a sequence complementary to the sequence of the above gene (1).

In a case where the above antisense oligonucleotide is used as a specific binding substance, for instance, when applied to an individual, the antisense oligonucleotide can be introduced into the individual by a local administration, a parenteral administration, an oral administration, or the like to a cancer tissue. The method of introduction includes a method for introduction using metal particles or a liposome, for instance, an inclusion form liposome, positively charged liposome, HVJ (Sendai virus)-liposome, modified HVJ-liposome (HVJ-AVE liposome), and the like; a method for direct introduction of naked DNA; a method for introduction with a positively charged polymer; and the like.

The antisense oligonucleotide can be used in the form of a solution in an appropriate solution, for instance, a buffer capable of stably maintaining an antisense oligonucleotide according to the purpose of use, mode of use, and the like. The antisense oligonucleotide may also be used as an agent comprising a pharmacologically acceptable carrier, an excipient, a binder, a stabilizer, a buffer, a solubilizing agent, an isotonic agent, or the like.

The above antisense oligonucleotide may be used in an amount sufficient to suppress proliferation of a cancer cell, and the dosage and the number of doses of the above antisense oligonucleotide can be appropriately set according to the purposes of administration, and the age, body weight, conditions, and the like of an individual, as long as an effect for suppressing proliferation of a cancer cell is sufficiently exhibited.

Also, the above antisense oligonucleotide may have various modifications capable of giving a characteristic suitable for its delivery to a cancer tissue to be targeted or affinity for a tissue or cell. Furthermore, the above antisense oligonucleotide may be supported by a carrier possessing a characteristic suitable for its delivery to the cancer tissue to be targeted or affinity for a tissue or cell.

In addition, a cytotoxic T lymphocyte can, for instance, be induced and obtained by repeatedly giving an antigen stimulation using a polypeptide encoded by the above cancer-associated gene to a lymphoblast from peripheral blood monocyte prepared from blood. The induced cytotoxic T lymphocyte can also be maintained as a lymphocyte possessing stable cytotoxicity by cloning. For instance, the lymphocyte can be proliferated by giving a stimulation to an induced CTL with an antigen, various cytokines, or an anti-CD3 antibody.

The antibody capable of specifically binding to the above polypeptide can be obtained in the same manner as that described above.

The protein or compound capable of specifically binding to the above polypeptide can be selected by a commonly used method for analyzing interactions between proteins, for instance, surface plasmon analysis; analysis based on changes in oscillation of a quartz oscillator to which the above polypeptide is immobilized; a method using an affinity column; and the like. In a case where a protein or compound capable of specifically binding to the above polypeptide is selected by surface plasmon analysis, a method, for instance, comprising the steps of:

supplying a protein-containing solution to be tested or a compound-containing solution to be tested at a given flow rate to a sensor chip to which a polypeptide encoded by the above cancer-associated gene is immobilized; and

detecting an interaction as an optical fluctuation or mass fluctuation by an appropriate detection means [for instance, an optical detection (degree of fluorescence, fluorescence polarization, or the like), combination with a mass spectrometer (matrix-assisted laser desorption ionization time-of-flight mass spectrometer: MALDI-TOF MS, electrospray ionization mass spectrometer: ESI-MS, and the like)],

can be carried out. Therefore, when a sensorgram showing the formation of a complex of the polypeptide and the protein to be tested or the compound to be tested is presented, a protein or compound capable of specifically binding to the above polypeptide can be selected by using as an index in a case where an optical sensorgram or mass sensorgram is fluctuated by introduction of the protein to be tested or the compound to be tested by liquid supply.

When the specific binding substance capable of specifically binding to the above (2) as the specific binding substance, when applied to an individual, the specific binding substance can be introduced to the individual, for instance, by a local administration, a parenteral administration, an oral administration, to a cancer tissue or the like.

The specific binding substance capable of specifically binding to the above (2) can also be used as an agent comprising a pharmacologically acceptable carrier, an excipient, a binder, a stabilizer, a buffer, a solubilizing agent, an isotonic agent, and the like.

The specific binding substance capable of specifically binding to the above (2) may be used in an amount a sufficient to suppress proliferation of a cancer cell, and the dosage and the number of doses thereof can be set appropriately according to the purpose of administration and the age, body weight, conditions, and the like of the individual, as long as an effect for suppressing proliferation of cancer cell can be sufficiently exhibited.

The present invention will be hereinafter described more specifically by means of Examples, without intending to limit the present invention to these examples.

### EXAMPLE 1

### Analysis of Cancer-Associated Genes

1) Confirmation of Presence of mRNA Which Can Be Used as Index of Cancer Detection

The differential display method by which canceration lesion portions are compared with control normal portions of the stomach was carried out to examine the presence or absence of mRNA of which expression level is altered due to canceration.

First, total RNA was extracted using TRIzol™ reagent (manufactured by Gibco-BRL) from each of a canceration lesion portion and a control normal portion of the stomach excised from a patient suffering from progressive conditions of poorly differentiated adenocarcinoma, to give a crude RNA sample. A 50 μg portion of the crude RNA sample obtained, MgCl₂ with a final concentration of 5 mM, 20 units of an RNase inhibitor (manufactured by Takara Shuzo Co., Ltd.) and 10 units of DNase I (manufactured by Takara Shuzo Co., Ltd.) were reacted at 37° C. for 30 minutes to remove genomic DNA, thereby giving an RNA sample.

For the above RNA sample, RT-PCR was carried out using the Fluorescence Differential Display™ Kit Rhodamine version (manufactured by Takara Shuzo Co., Ltd.) and Enzyme Set-FDD (manufactured by Takara Shuzo Co., Ltd.) in accordance with the instruction manuals attached to the kits.

When the reverse transcription reaction in RT-PCR is carried out, there were mixed 200 μg of the above RNA sample and a rhodamine-labeled downstream primer having a 5′-CG-3′ anchor sequence attached to the above Fluorescence Differential Display™ Kit Rhodamine version, to give a mixture. Subsequently, the mixture was heat-treated at 70° C. for 10 minutes and rapidly cooled to give a reaction product. Thereafter, the resulting reaction product and AMV reverse transcriptase were mixed, and the reverse transcription reaction was carried out at 55° C. for 30 minutes to give a single-stranded cDNA sample.

PCR was carried out in a reaction mixture containing 1.3 mM MgCl₂ and 100 μM each of dATP, dGTP, dCTP and dTTP as substrates by using the same rhodamine-labeled downstream primer as that used for the reverse transcription reaction and any one kind out of the 24 kinds of upstream primers (R1-R24) in the above kit with the above single-stranded cDNA sample as a template, to give a total of 24 kinds of amplified DNA samples.

The thermal profile of the above PCR is as follows:

94° C. for 2 minutes, 40° C. for 5 minutes, 72° C. for 5 minutes;

thereafter 34 cycles of reaction, wherein one cycle is 94° C. for 30 seconds, 40° C. for 2 minutes, and 72° C. for 1 minute;

subsequently at 72° C. for 5 minutes.

After the termination of the reaction, 95% formamide was added in an equivolume to the resulting reaction product, and the reaction product was thermally denatured at 90° C. for 2 minutes to give a sample for electrophoresis. The electrophoresis was carried out on a 7 M urea-denatured 4% polyacrylamide gel. The migration pattern on the gel obtained was read off and detected with a fluorescence image analyzer FMBIO® II Multi-View (manufactured by Takara Shuzo Co., Ltd.). As a result, a fingerprint comprising a large number of bands was obtained, in which there existed bands showing difference in intensity between the cancer lesion portion and the control normal portion.

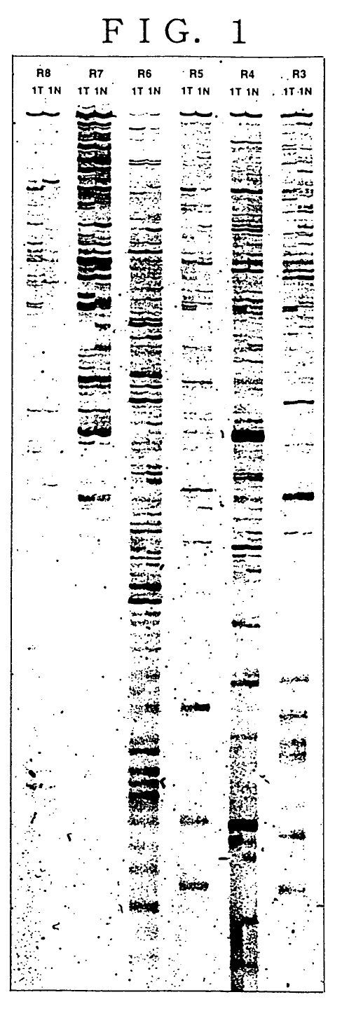

As one example of the above fingerprint, the results in which the upstream primers R3 to R8 were used are shown in FIG. 1.

Accordingly, FIG. 1 shows a fingerprint of the electrophoretic patterns for genes evaluated showing expression differences between a cancer tissue and a control normal tissue by the DD method. In FIG. 1, 1N denotes a migration lane for an amplified DNA fragment obtained using as a template an RNA sample from a normal tissue portion of a patient suffering from poorly differentiated adenocarcinoma type gastric cancer, and 1T denotes a migration lane for an amplified DNA fragment obtained using as a template an RNA sample from a cancer tissue portion of the same patient suffering from poorly differentiated adenocarcinoma type gastric cancer.

Next, a polyacrylamide gel plate after the electrophoresis was placed on the fingerprint obtained, and 250 bands showing difference in intensity between the cancer lesion portion and the control normal portion were cut out. DNA fragments were extracted with water from the gel, and amplification was again carried out by PCR using the primers corresponding to each of the DNA fragments in Cloning-Sequencing Primer Set for FDD (manufactured by Takara Shuzo Co., Ltd.).

The amplified fragments were cloned by TA cloning, and isolated four clones each for one fragment. Subsequently, the nucleotide sequences were determined for the nucleic acids contained in the clones obtained by the dideoxy method. The nucleotide sequences obtained were assembled by using the Paracel Clustering package software (manufactured by Paracel), and homology search was carried out for each contig sequence formed using a database of nucleotide sequence information. The results are shown in Tables 1 and 2 and Tables 3 to 5.

TABLE 1

| SEQ ID | Clone | | Accession |
|---|---|---|---|
| NO | No. | Name of Gene | No |
| 1 | 20 | H. sapiens mRNA for RING protein | Y07828 |
| 2 | 42 | Homo sapiens mRNA for PKU-beta, com | AB004885 |
| 3 | 49 | Homo sapiens carboxy terminus of Hsp70-interacting protein | AF129085 |
| | | (CHIP) mRNA, complete cds | |
| 4 | 68 | Homo sapiens mRNA; cDNA DKFZp586I0523 (from clone | AL050217 |
| | | DKFZp586I0523) | |
| 5 | 95 | H. sapiens Sp17 gene | Z48570 |
| 6 | 129 | Homo sapiens MD-1 mRNA, complete cds | AF057178 |
| 7 | 132 | H. sapiens mRNA for BiP protein | X87949 |
| 8 | 155 | Human chromosome 17q12-21 mRNA, clone pOV-2, partial cds | U18919 |
| 9 | 203 | Homo sapiens dead box, X isoform (DBX) mRNA, alternative | AF000982 |
| | | transcript 2, complete cds | |
| 10 | 291 | Human ferritin heavy chain nRNA, complete cds | M97164 |
| 11 | 295 | Human ovarian cancer downregulated myosin heavy chain homolog | U53445 |
| | | (Doc1) mRNA, complete cds | |
| 12 | 323 | Homo sapiens full length insert cDNA clone YP42A04 | AF085884 |
| 13 | 337 | Human mRNA for protein disulfide isomerase-related protein P5, | D49489 |
| | | complete cds | |
| 14 | 344 | Homo sapiens Ku70-binding protein (KUB3) mRNA, partial cds | AF078164 |
| 15 | 354 | Human nonmuscle myosin heavy chain-B (MYH10) mRNA, partial cds | M69181 |
| 16 | 377 | Human voltage-dependent anion channel isoform 1 (VDAC) mRNA, | L06132 |
| | | complete cds | |
| 17 | 382 | Homo sapiens phosphatidylinositol 4-kinase mRNA, complete cds | L36151 |
| 18 | 383 | Human beta adaptin mRNA, complete cds | M34175 |
| 19 | 386 | Homo sapiens H beta 58 homolog mRNA, complete cds | AF054179 |
| 20 | 394 | Homo sapiens unknown mRNA, complete cds | AF047439 |
| 21 | 426 | Homo sapiens NRD convertase mRNA, complete cds | U64898 |
| 22 | 430 | H. sapiens mRNA for acylphosphatase, muscle type (MT) isoenzyme | X84195 |
| 23 | 436 | Homo sapiens transmembrane protein BRI (BRI) | AF152462 |
| 24 | 449 | Homo sapiens dynactin subunit (p22) mRNA, complete cds | AF082513 |
| 25 | 461 | Human mRNA for aldose reductase (EC 1.1.1.2) | X15414 |
| 26 | 463 | Homo sapiens cDNA FLJ20693 fis, clone | AK000700 |
| 27 | 503 | Homo sapiens mRNA for scrapie respnsive protein 1o | AJ224677 |
| 28 | 507 | Homo sapiens KIAA0402 mRNA, partial cds | AB007862 |
| 29 | 511 | Human transactivator protein (CREB) mRNA, complete cds | M27691 |
| 30 | 522 | Human MAL protein gene mRNA, complete cds | M15800 |
| 31 | 552 | Homo sapiens ataxin-2-like protein A 2LP (A2LG) mRNA, complete | AF034373 |
| | | cds | |
| 32 | 556 | Human phosphatidylinositol transfer protein mRNA, complete cds | M73704 |
| 33 | 561 | H. sapiens mRNA for transketolase | X67688 |
| 34 | 584 | Homo sapiens nibrin (NBS) mRNA, complete cds | AF051334 |

TABLE 2

| SEQ ID | Clone | | Accession |
|---|---|---|---|
| NO | No. | Name of Gene | No |
| 35 | 589 | Human SnRNP core protein Sm D3 mRNA, complete cds | U15009 |
| 36 | 602 | Homo sapiens FUS/TLS protein gene. | AF071213 |
| 37 | 609 | Homo sapiens clone 486790 diphosphoinositol polyphosphate | AF062529 |
| | | phosphohydrolase mRNA, complete cds | |
| 38 | 629 | Human globin gene | M69023 |
| 39 | 645 | H. sapiens mRNA for aminopeptidase P-like | X95762 |
| 40 | 668 | H. sapiens mRNA for UDP-GalNAc: polypeptide N- | X92689 |
| | | acetylgalactosaminyl transferase | |
| 41 | 723 | Homo sapiens cDNA FLJ20463 fis, clone | AK000470 |
| 42 | 731 | Novel gene similar to C. elegens hypothetical 55.2 KD protein | AL050255 |
| | | F16A11.2, SW: P90838 | |
| 43 | 766 | Homo sapiens cDNA FLJ10986 fis, clone | AK001848 |
| 44 | 778 | Human inhibitor of apoptosis protein 2 mRNA, complete cds | U45879 |
| 45 | 801 | Human MB-1 mRNA, complete cds | M80462 |
| 46 | 802 | Human tissue factor gene, complete cds. 1/1995 | J 02846 |
| 47 | 817 | Homo sapiens mRNA: cDNA DKFZp566E0224 (from clone | AL050031 |
| | | DKFZp566E0224) | |
| 48 | 818 | Homo sapiens NADH-ubiquinone oxidoreductase subunit CI-B12 | AF047183 |
| | | mRNA, complete cds | |
| 49 | 839 | Homo sapiens ASH1 mRNA, complete cds. 5/2000 | AF257305 |
| 50 | 845 | Homo sapiens CGI-65 protein mRNA, complete cds | AF151823 |
| 51 | 854 | Homo sapiens clone 24451 mRNA sequence | AF070599 |
| 52 | 877 | Homo sapiens ubiquitin hydrolyzing enzyme I (UBH1) mRNA, | AF022789 |
| | | partial cds | |
| 53 | 882 | Homo sapiens mRNA for cysteine-rich protein | AJ006591 |
| 54 | 883 | Human mRNA for KIAA0139 gene, complete cds | D50929 |
| 55 | 925 | Homo sapiens clone 23819 white protein homolog mRNA, partial | AF038175 |
| | | cds | |
| 56 | 944 | Homo sapiens connexin 26 (GJB2) mRNA, complete cds | M86849 |
| 57 | 1001 | Homo sapiens mRNA for KIAA0914 protein, complete cds | AB020721 |
| 58 | 1008 | Homo sapiens mRNA for KIAA0907 protein, complete cds | AB020714 |
| 59 | 1014 | H. sapiens mRNA for transcriptional intermediary factor 2 | X97674 |
| 60 | 1018 | Human cytosolic aspartate aminotransferase mRNA, complete cds | M37400 |
| 61 | 1023 | Homo sapiens CGI-118 protein mRNA, complete cds | AF151876 |
| 62 | 1028 | Human guanylate binding protein isoform II (GBP-2) mRNA, | M55543 |
| | | complete cds | |
| 63 | 1043 | Homo sapiens cDNA FLJ10633 fis. clone | AK001495 |
| 64 | 1046 | Homo sapiens chaperonin containing t-complex polypeptide 1, | AF026292 |
| | | eta subunit (Ccth) mRNA, complete cds | |
| 65 | 1060 | Homo sapiens mRNA for Prer protein | AJ005579 |

It was clarified from these results that the 65 genes shown in Tables 1 and 2 (SEQ ID NOs: 1 to 65) had been already isolated and identified genes.

Tables 3 to 5 show SEQ ID NOs and clone numbers of the nucleotide sequences of the fragments found to show differential expression according to the DD method, names for the upstream primers used for detecting the fragments in the DD method, approximate sizes of the amplified DNA fragments, and difference in the amount of amplified DNA between the cancer lesion portion and the control normal portion. The numbers in the primer columns of Tables 3 to 5 show the names for the upstream primers in the kit.

TABLE 3

| | | | Size of | |
|---|---|---|---|---|
| | | | Amplified | |
| SEQ | | | DNA | |
| ID | Clone | Upstream | Fragment | Difference in Amount of |
| NO | No. | Primer | (bp) | Amplified DNA |
| 1 | 20 | R20 | 320 | Cancer Tissue > Normal Tissue |
| 2 | 42 | R21 | 545 | Cancer Tissue < Normal Tissue |
| 3 | 49 | R22 | 650 | Cancer Tissue < Normal Tissue |
| 4 | 68 | R23 | 870 | Cancer Tissue < Normal Tissue |
| 5 | 95 | R23 | 550 | Cancer Tissue > Normal Tissue |
| 6 | 129 | R24 | 600 | Cancer Tissue < Normal Tissue |
| 7 | 132 | R2 | 930 | Cancer Tissue > Normal Tissue |
| 8 | 155 | R4 | 525 | Cancer Tissue < Normal Tissue |
| 9 | 203 | R10 | 280 | Cancer Tissue < Normal Tissue |
| 10 | 291 | R19 | 505 | Cancer Tissue > Normal Tissue |
| 11 | 295 | R20 | 1010 | Cancer Tissue < Normal Tissue |
| 12 | 323 | R2 | 510 | Cancer Tissue < Normal Tissue |
| 13 | 337 | R2 | 910 | Cancer Tissue > Normal Tissue |
| 14 | 344 | R2 | 795 | Cancer Tissue > Normal Tissue |
| 15 | 354 | R2 | 570 | Cancer Tissue > Normal Tissue |
| 16 | 377 | R2 | 480 | Cancer Tissue > Normal Tissue |
| 17 | 382 | R3 | 400 | Cancer Tissue < Normal Tissue |
| 18 | 383 | R3 | 390 | Cancer Tissue < Normal Tissue |
| 19 | 386 | R3 | 350 | Cancer Tissue > Normal Tissue |
| 20 | 394 | R3 | 570 | Cancer Tissue > Normal Tissue |
| 21 | 426 | R4 | 345 | Cancer Tissue < Normal Tissue |
| 22 | 430 | R4 | 310 | Cancer Tissue < Normal Tissue |
| 23 | 436 | R4 | 860 | Cancer Tissue < Normal Tissue |
| 24 | 449 | R4 | 480 | Cancer Tissue < Normal Tissue |
| 25 | 461 | R4 | 535 | Cancer Tissue > Normal Tissue |

TABLE 4

| | | | Size of | |
|---|---|---|---|---|
| | | | Amplified | |
| SEQ | | | DNA | |
| ID | Clone | Upstream | Fragment | Difference in Amount of |
| NO | No. | Primer | (bp) | Amplified DNA |
| 26 | 463 | R4 | 505 | Cancer Tissue < Normal Tissue |
| 27 | 503 | R5 | 445 | Cancer Tissue > Normal Tissue |
| 28 | 507 | R5 | 950 | Cancer Tissue > Normal Tissue |
| 29 | 511 | R5 | 560 | Cancer Tissue < Normal Tissue |
| 30 | 522 | R6 | 250 | Cancer Tissue < Normal Tissue |
| 31 | 552 | R6 | 680 | Cancer Tissue < Normal Tissue |
| 32 | 556 | R6 | 650 | Cancer Tissue > Normal Tissue |
| 33 | 561 | R6 | 470 | Cancer Tissue > Normal Tissue |
| 34 | 584 | R8 | 625 | Cancer Tissue > Normal Tissue |
| 35 | 589 | R9 | 420 | Cancer Tissue > Normal Tissue |
| 36 | 602 | R9 | 280 | Cancer Tissue < Normal Tissue |
| 37 | 609 | R9 | 270 | Cancer Tissue < Normal Tissue |
| 38 | 629 | R9 | 465 | Cancer Tissue > Normal Tissue |
| 39 | 645 | R9 | 1200 | Cancer Tissue < Normal Tissue |
| 40 | 668 | R9 | 690 | Cancer Tissue < Normal Tissue |
| 41 | 723 | R10 | 650 | Cancer Tissue < Normal Tissue |
| 42 | 731 | R10 | 560 | Cancer Tissue < Normal Tissue |
| 43 | 766 | R11 | 510 | Cancer Tissue < Normal Tissue |
| 44 | 778 | R12 | 360 | Cancer Tissue > Normal Tissue |
| 45 | 801 | R12 | 500 | Cancer Tissue > Normal Tissue |
| 46 | 802 | R12 | 395 | Cancer Tissue < Normal Tissue |
| 47 | 817 | R12 | 310 | Cancer Tissue < Normal Tissue |
| 48 | 818 | R12 | 320 | Cancer Tissue < Normal Tissue |
| 49 | 839 | R13 | 580 | Cancer Tissue < Normal Tissue |
| 50 | 845 | R13 | 480 | Cancer Tissue < Normal Tissue |

TABLE 5

| | | | Size of | |
|---|---|---|---|---|
| | | | Amplified | |
| SEQ | | | DNA | |
| ID | Clone | Upstream | Fragment | Difference in Amount of |
| NO | No. | Primer | (bp) | Amplified DNA |
| 51 | 854 | R13 | 360 | Cancer Tissue < Normal Tissue |
| 52 | 877 | R13 | 270 | Cancer Tissue > Normal Tissue |
| 53 | 882 | R13 | 940 | Cancer Tissue > Normal Tissue |
| 54 | 883 | R13 | 385 | Cancer Tissue > Normal Tissue |
| 55 | 925 | R15 | 330 | Cancer Tissue < Normal Tissue |
| 56 | 944 | R16 | 390 | Cancer Tissue > Normal Tissue |
| 57 | 1001 | R16 | 310 | Cancer Tissue < Normal Tissue |
| 58 | 1008 | R17 | 290 | Cancer Tissue > Normal Tissue |
| 59 | 1014 | R17 | 210 | Cancer Tissue > Normal Tissue |
| 60 | 1018 | R17 | 730 | Cancer Tissue < Normal Tissue |
| 61 | 1023 | R17 | 610 | Cancer Tissue > Normal Tissue |
| 62 | 1028 | R18 | 550 | Cancer Tissue > Normal Tissue |
| 63 | 1043 | R18 | 615 | Cancer Tissue > Normal Tissue |
| 64 | 1046 | R19 | 350 | Cancer Tissue > Normal Tissue |
| 65 | 1060 | R20 | 625 | Cancer Tissue < Normal Tissue |

Further, in addition to the above genes, 199 kinds of gene fragments which were not found to have homology to a known gene were obtained by homology search using the database. The nucleotide sequences read off for these cancer-associated gene fragments are shown in SEQ ID NOs: 66 to 264. In addition, Tables 6 to 14 show SEQ ID NOs and clone numbers of the nucleotide sequences of the above cancer-associated gene fragments, names for the upstream primers used for detecting the fragments in the DD method, and difference in the amount of amplified DNA between the cancer lesion portion and the control normal portion. The numbers in the primer columns of Tables 6 to 14 show the names for the upstream primers in the kit.

TABLE 6

| | Clone | Upstream | Difference in Amount of |
|---|---|---|---|
| SEQ ID NO | No. | Primer | Amplified DNA |
| 66 | 3 | R20 | Cancer Tissue < Normal Tissue |
| 67 | 4 | R20 | Cancer Tissue < Normal Tissue |
| 68 | 492 | R5 | Cancer Tissue < Normal Tissue |
| 69 | 502 | R5 | Cancer Tissue < Normal Tissue |
| 70 | 506 | R5 | Cancer Tissue < Normal Tissue |
| 71 | 104 | R23 | Cancer Tissue < Normal Tissue |
| 72 | 515 | R5 | Cancer Tissue < Normal Tissue |
| 73 | 517 | R6 | Cancer Tissue < Normal Tissue |
| 74 | 520 | R6 | Cancer Tissue < Normal Tissue |
| 75 | 57 | R23 | Cancer Tissue < Normal Tissue |
| 76 | 534 | R6 | Cancer Tissue < Normal Tissue |
| 77 | 535 | R6 | Cancer Tissue < Normal Tissue |
| 78 | 59 | R23 | Cancer Tissue < Normal Tissue |
| 79 | 564 | R7 | Cancer Tissue < Normal Tissue |
| 80 | 566 | R7 | Cancer Tissue < Normal Tissue |
| 81 | 573 | R7 | Cancer Tissue < Normal Tissue |
| 82 | 581 | R8 | Cancer Tissue > Normal Tissue |
| 83 | 585 | R9 | Cancer Tissue < Normal Tissue |
| 84 | 596 | R9 | Cancer Tissue < Normal Tissue |
| 85 | 608 | R9 | Cancer Tissue < Normal Tissue |
| 86 | 65 | R23 | Cancer Tissue < Normal Tissue |
| 87 | 614 | R9 | Cancer Tissue > Normal Tissue |
| 88 | 615 | R9 | Cancer Tissue < Normal Tissue |
| 89 | 625 | R9 | Cancer Tissue < Normal Tissue |

TABLE 7

| | Clone | Upstream | Difference in Amount of |
|---|---|---|---|
| SEQ ID NO | No. | Primer | Amplified DNA |
| 90 | 680 | R9 | Cancer Tissue < Normal Tissue |
| 91 | 144 | R9 | Cancer Tissue < Normal Tissue |
| 92 | 640 | R9 | Cancer Tissue < Normal Tissue |
| 93 | 305 | R9 | Cancer Tissue < Normal Tissue |
| 94 | 648 | R9 | Cancer Tissue < Normal Tissue |
| 95 | 649 | R9 | Cancer Tissue < Normal Tissue |
| 96 | 657 | R9 | Cancer Tissue < Normal Tissue |
| 97 | 660 | R9 | Cancer Tissue < Normal Tissue |
| 98 | 7 | R20 | Cancer Tissue < Normal Tissue |
| 99 | 671 | R9 | Cancer Tissue < Normal Tissue |
| 100 | 675 | R9 | Cancer Tissue < Normal Tissue |
| 101 | 694 | R10 | Cancer Tissue < Normal Tissue |
| 102 | 75 | R23 | Cancer Tissue < Normal Tissue |
| 103 | 701 | R10 | Cancer Tissue < Normal Tissue |
| 104 | 705 | R10 | Cancer Tissue < Normal Tissue |
| 105 | 712 | R10 | Cancer Tissue > Normal Tissue |
| 106 | 719 | R10 | Cancer Tissue < Normal Tissue |
| 107 | 1027 | R17 | Cancer Tissue < Normal Tissue |
| 108 | 1033 | R18 | Cancer Tissue < Normal Tissue |
| 109 | 77 | R23 | Cancer Tissue < Normal Tissue |
| 110 | 1036 | R18 | Cancer Tissue < Normal Tissue |
| 111 | 1038 | R18 | Cancer Tissue < Normal Tissue |
| 112 | 737 | R10 | Cancer Tissue < Normal Tissue |
| 113 | 781 | R12 | Cancer Tissue < Normal Tissue |

TABLE 8

| | Clone | Upstream | Difference in Amount of |
|---|---|---|---|
| SEQ ID NO | No. | Primer | Amplified DNA |
| 114 | 786 | R12 | Cancer Tissue < Normal Tissue |
| 115 | 807 | R12 | Cancer Tissue < Normal Tissue |
| 116 | 810 | R12 | Cancer Tissue < Normal Tissue |
| 117 | 860 | R13 | Cancer Tissue < Normal Tissue |
| 118 | 886 | R14 | Cancer Tissue > Normal Tissue |
| 119 | 887 | R14 | Cancer Tissue < Normal Tissue |
| 120 | 892 | R14 | Cancer Tissue < Normal Tissue |
| 121 | 897 | R14 | Cancer Tissue < Normal Tissue |
| 122 | 894 | R14 | Cancer Tissue < Normal Tissue |
| 123 | 912 | R14 | Cancer Tissue < Normal Tissue |
| 124 | 916 | R15 | Cancer Tissue > Normal Tissue |
| 125 | 928 | R16 | Cancer Tissue < Normal Tissue |
| 126 | 929 | R16 | Cancer Tissue < Normal Tissue |
| 127 | 933 | R16 | Cancer Tissue < Normal Tissue |
| 128 | 983 | R16 | Cancer Tissue < Normal Tissue |
| 129 | 938 | R16 | Cancer Tissue < Normal Tissue |
| 130 | 941 | R16 | Cancer Tissue < Normal Tissue |
| 131 | 945 | R16 | Cancer Tissue > Normal Tissue |
| 132 | 952 | R16 | Cancer Tissue < Normal Tissue |
| 133 | 951 | R16 | Cancer Tissue < Normal Tissue |
| 134 | 954 | R16 | Cancer Tissue < Normal Tissue |
| 135 | 85 | R23 | Cancer Tissue < Normal Tissue |
| 136 | 960 | R16 | Cancer Tissue < Normal Tissue |
| 137 | 963 | R16 | Cancer Tissue < Normal Tissue |

TABLE 9

| | Clone | Upstream | Difference in Amount of |
|---|---|---|---|
| SEQ ID NO | No. | Primer | Amplified DNA |
| 138 | 968 | R16 | Cancer Tissue < Normal Tissue |
| 139 | 974 | R16 | Cancer Tissue < Normal Tissue |
| 140 | 977 | R16 | Cancer Tissue < Normal Tissue |
| 141 | 988 | R16 | Cancer Tissue < Normal Tissue |
| 142 | 990 | R16 | Cancer Tissue < Normal Tissue |
| 143 | 991 | R16 | Cancer Tissue < Normal Tissue |
| 144 | 1045 | R19 | Cancer Tissue < Normal Tissue |
| 145 | 1052 | R19 | Cancer Tissue < Normal Tissue |
| 146 | 92 | R23 | Cancer Tissue < Normal Tissue |
| 147 | 743 | R11 | Cancer Tissue < Normal Tissue |
| 148 | 771 | R11 | Cancer Tissue < Normal Tissue |
| 149 | 752 | R11 | Cancer Tissue < Normal Tissue |
| 150 | 755 | R11 | Cancer Tissue < Normal Tissue |
| 151 | 761 | R11 | Cancer Tissue < Normal Tissue |
| 152 | 762 | R11 | Cancer Tissue < Normal Tissue |
| 153 | 765 | R11 | Cancer Tissue < Normal Tissue |
| 154 | 775 | R11 | Cancer Tissue < Normal Tissue |
| 155 | 792 | R12 | Cancer Tissue < Normal Tissue |
| 156 | 793 | R12 | Cancer Tissue < Normal Tissue |
| 157 | 797 | R12 | Cancer Tissue > Normal Tissue |
| 158 | 119 | R23 | Cancer Tissue < Normal Tissue |
| 159 | 816 | R12 | Cancer Tissue < Normal Tissue |
| 160 | 819 | R12 | Cancer Tissue < Normal Tissue |
| 161 | 822 | R12 | Cancer Tissue < Normal Tissue |

TABLE 10

| | Clone | Upstream | Difference in Amount of |
|---|---|---|---|
| SEQ ID NO | No. | Primer | Amplified DNA |
| 162 | 825 | R12 | Cancer Tissue < Normal Tissue |
| 163 | 838 | R12 | Cancer Tissue < Normal Tissue |
| 164 | 855 | R13 | Cancer Tissue < Normal Tissue |
| 165 | 847 | R13 | Cancer Tissue < Normal Tissue |
| 166 | 866 | R13 | Cancer Tissue < Normal Tissue |
| 167 | 103 | R23 | Cancer Tissue < Normal Tissue |
| 168 | 875 | R13 | Cancer Tissue < Normal Tissue |
| 169 | 880 | R13 | Cancer Tissue < Normal Tissue |
| 170 | 899 | R14 | Cancer Tissue < Normal Tissue |
| 171 | 902 | R14 | Cancer Tissue < Normal Tissue |
| 172 | 908 | R14 | Cancer Tissue < Normal Tissue |
| 173 | 325 | R2 | Cancer Tissue < Normal Tissue |
| 174 | 416 | R4 | Cancer Tissue < Normal Tissue |
| 175 | 420 | R4 | Cancer Tissue < Normal Tissue |
| 176 | 451 | R4 | Cancer Tissue < Normal Tissue |
| 177 | 452 | R4 | Cancer Tissue < Normal Tissue |
| 178 | 302 | R20 | Cancer Tissue < Normal Tissue |
| 179 | 121 | R23 | Cancer Tissue < Normal Tissue |
| 180 | 122 | R24 | Cancer Tissue < Normal Tissue |
| 181 | 137 | R21 | Cancer Tissue < Normal Tissue |
| 182 | 15 | R20 | Cancer Tissue < Normal Tissue |
| 183 | 161 | R9 | Cancer Tissue < Normal Tissue |
| 184 | 173 | R10 | Cancer Tissue < Normal Tissue |
| 185 | 21 | R20 | Cancer Tissue > Normal Tissue |

TABLE 11

| | Clone | Upstream | Difference in Amount of |
|---|---|---|---|
| SEQ ID NO | No. | Primer | Amplified DNA |
| 186 | 287 | R18 | Cancer Tissue < Normal Tissue |
| 187 | 301 | R20 | Cancer Tissue < Normal Tissue |
| 188 | 310 | R17 | Cancer Tissue < Normal Tissue |
| 189 | 554 | R6 | Cancer Tissue < Normal Tissue |
| 190 | 749 | R11 | Cancer Tissue > Normal Tissue |
| 191 | 1061 | R20 | Cancer Tissue < Normal Tissue |
| 192 | 1062 | R20 | Cancer Tissue < Normal Tissue |
| 193 | 316 | R2 | Cancer Tissue < Normal Tissue |
| 194 | 318 | R2 | Cancer Tissue < Normal Tissue |
| 195 | 320 | R2 | Cancer Tissue < Normal Tissue |
| 196 | 322 | R2 | Cancer Tissue < Normal Tissue |
| 197 | 329 | R2 | Cancer Tissue > Normal Tissue |
| 198 | 330 | R2 | Cancer Tissue < Normal Tissue |
| 199 | 335 | R2 | Cancer Tissue < Normal Tissue |
| 200 | 339 | R2 | Cancer Tissue < Normal Tissue |
| 201 | 345 | R2 | Cancer Tissue < Normal Tissue |
| 202 | 354 | R2 | Cancer Tissue > Normal Tissue |
| 203 | 367 | R2 | Cancer Tissue > Normal Tissue |
| 204 | 357 | R2 | Cancer Tissue < Normal Tissue |
| 205 | 363 | R2 | Cancer Tissue < Normal Tissue |
| 206 | 362 | R2 | Cancer Tissue < Normal Tissue |
| 207 | 370 | R2 | Cancer Tissue < Normal Tissue |
| 208 | 371 | R2 | Cancer Tissue < Normal Tissue |
| 209 | 372 | R2 | Cancer Tissue < Normal Tissue |

TABLE 12

| | Clone | Upstream | Difference in Amount of |
|---|---|---|---|
| SEQ ID NO | No. | Primer | Amplified DNA |
| 210 | 387 | R3 | Cancer Tissue < Normal Tissue |
| 211 | 392 | R3 | Cancer Tissue < Normal Tissue |
| 212 | 398 | R3 | Cancer Tissue < Normal Tissue |
| 213 | 405 | R3 | Cancer Tissue < Normal Tissue |
| 214 | 407 | R3 | Cancer Tissue < Normal Tissue |
| 215 | 427 | R4 | Cancer Tissue < Normal Tissue |
| 216 | 435 | R4 | Cancer Tissue < Normal Tissue |
| 217 | 429 | R4 | Cancer Tissue < Normal Tissue |
| 218 | 441 | R4 | Cancer Tissue < Normal Tissue |
| 219 | 51 | R23 | Cancer Tissue < Normal Tissue |
| 220 | 465 | R4 | Cancer Tissue < Normal Tissue |
| 221 | 468 | R4 | Cancer Tissue < Normal Tissue |
| 222 | 471 | R4 | Cancer Tissue < Normal Tissue |
| 223 | 498 | R5 | Cancer Tissue < Normal Tissue |
| 224 | 44 | R21 | Cancer Tissue < Normal Tissue |
| 225 | 68 | R23 | Cancer Tissue < Normal Tissue |
| 226 | 124 | R24 | Cancer Tissue < Normal Tissue |
| 227 | 133 | R2 | Cancer Tissue < Normal Tissue |
| 228 | 300 | R20 | Cancer Tissue < Normal Tissue |
| 229 | 315 | R2 | Cancer Tissue < Normal Tissue |
| 230 | 323 | R2 | Cancer Tissue < Normal Tissue |
| 231 | 337 | R2 | Cancer Tissue > Normal Tissue |
| 232 | 342 | R2 | Cancer Tissue < Normal Tissue |
| 233 | 347 | R2 | Cancer Tissue < Normal Tissue |

TABLE 13

| | Clone | Upstream | Difference in Amount of |
|---|---|---|---|
| SEQ ID NO | No. | Primer | Amplified DNA |
| 234 | 404 | R3 | Cancer Tissue < Normal Tissue |
| 235 | 419 | R4 | Cancer Tissue < Normal Tissue |
| 236 | 455 | R4 | Cancer Tissue < Normal Tissue |
| 237 | 456 | R4 | Cancer Tissue < Normal Tissue |
| 238 | 458 | R4 | Cancer Tissue > Normal Tissue |
| 239 | 503 | R5 | Cancer Tissue > Normal Tissue |
| 240 | 529 | R6 | Cancer Tissue < Normal Tissue |
| 241 | 543 | R6 | Cancer Tissue < Normal Tissue |
| 242 | 589 | R9 | Cancer Tissue > Normal Tissue |
| 243 | 609 | R9 | Cancer Tissue < Normal Tissue |
| 244 | 620 | R9 | Cancer Tissue > Normal Tissue |
| 245 | 621 | R9 | Cancer Tissue < Normal Tissue |
| 246 | 654 | R9 | Cancer Tissue > Normal Tissue |
| 247 | 666 | R9 | Cancer Tissue < Normal Tissue |
| 248 | 667 | R9 | Cancer Tissue < Normal Tissue |
| 249 | 714 | R10 | Cancer Tissue < Normal Tissue |
| 250 | 738 | R11 | Cancer Tissue < Normal Tissue |
| 251 | 751 | R11 | Cancer Tissue < Normal Tissue |
| 252 | 753 | R11 | Cancer Tissue < Normal Tissue |
| 253 | 806 | R12 | Cancer Tissue < Normal Tissue |
| 254 | 828 | R12 | Cancer Tissue < Normal Tissue |
| 255 | 840 | R13 | Cancer Tissue < Normal Tissue |
| 256 | 849 | R13 | Cancer Tissue < Normal Tissue |
| 257 | 869 | R13 | Cancer Tissue > Normal Tissue |

TABLE 14

| | Clone | Upstream | Difference in Amount of |
|---|---|---|---|
| SEQ ID NO | No. | Primer | Amplified DNA |
| 258 | 917 | R15 | Cancer Tissue < Normal Tissue |
| 259 | 1056 | R19 | Cancer Tissue < Normal Tissue |
| 260 | 1013 | R17 | Cancer Tissue < Normal Tissue |
| 261 | 578 | R8 | Cancer Tissue < Normal Tissue |
| 262 | 768 | R11 | Cancer Tissue < Normal Tissue |
| 263 | 70 | R23 | Cancer Tissue < Normal Tissue |
| 264 | 123 | R24 | Cancer Tissue > Normal Tissue |

2) Association of the Above Group of Genes in Canceration

(1) Preparation of DNA Array

Whether the alterations in expression levels of mRNA used as a template for the amplified DNA fragments from each of the genes shown in Tables 3 to 14, as confirmed by using the DD method as described in the above 1), is truly associated with canceration was studied by using a DNA array.

First, the desired cDNA fragment was amplified by PCR method by using as templates primers set on both ends of the multicloning site of the plasmid with 340 kinds of clones each comprising each of sequences representing respective contigs, out of the clones detected by the DD method according to the method described in the above 1). Thereafter, the nucleotide sequence analysis for the amplified cDNA fragment was carried out to confirm that an amplified fragment is a desired fragment. Also, the desired fragment was subjected to ethanol precipitation and collected, and the collected fragments were dissolved in 100 mM carbonate buffer (pH 9.5) so as to have a final concentration of 1 μM.

Besides the above, β-actin gene, genes of tubulin α2, cyclophilin, glycelaldehyde 3-phosphate dehydrogenase, ribosomal protein S5, ribosomal protein S9 and the like as housekeeping genes, and plasmid pUC18 as negative control were prepared in the same manner genes, respectively. Each of these was spotted onto an amino-group introduced slide glass (manufactured by Sigma) by using an apparatus for preparing a DNA chip [Affymtrix® 417™ arrayer, manufactured by Affymetrix], and fixed by UV irradiation. The slide was washed with 0.2% SDS, then with distilled water, and dried to give a DNA array.

(2) Preparation of Fluorescence-Labeled cDNA

Gastric cancer tissue and control normal gastric tissue were separated from tissues excised during cancer excision surgery from 54 patients with a gastric cancer of different degrees of progress. Subsequently, total RNA was extracted from each tissue by the AGPC (Acid Guanidium Phenol-Chloroform) method. mRNA was purified from each of these total RNAs using Oligotex-MAG mRNA Purification Kit (manufactured by Takara Shuzo Co., Ltd.).

cDNA synthesis reaction was carried out by using a reverse transcriptase with the above mRNA as a template. In a case of a control normal gastric tissue group, dNTP containing Cy3-dUTP (manufactured by Amersham) was used; and in a case of a gastric cancer tissue group, dNTP containing

Cy5-dUTP (manufactured by Amersham) was used. The compositions for the reaction mixtures are as follows.

Reaction mixture A: About 1 μg of the above mRNA, 300 pmol of oligo-dT primer (manufactured by Takara Shuzo Co., Ltd.) and diethyl pyrrocarbonate (DEPC, manufactured by Nacalai Tesque Inc.)-treated water being added thereto to make up a final volume of 11.9 μl.

Reaction mixture B: 4 μl of 5×AMV RTase buffer (manufactured by Life Science), 0.1 mM each of dATP, dCTP and dGTP, 0.065 mM dTTP, 30 U of an RNase inhibitor (manufactured by Takara Shuzo Co., Ltd.), and 0.035 mM Cy3- or Cy5-labeled dUTP (manufactured by Amersham-Pharmacia) were mixed to give a solution with a final volume of 6.5 μl.

The reaction mixture A was kept at 70° C. for 10 minutes, and thereafter cooled on an ice bath to give a reaction product. Next, the reaction mixture B and about 30 units of AMV RTase (manufactured by Life Science) were added to the above reaction product, and this mixture was kept at 55° C. for 30 minutes. Thereafter, about 30 units of AMV RTase was additionally added to the resulting reaction product to make up a volume of 20 μl as the volume of reaction mixture, to give an RT reaction mixture. The above RT reaction mixture was kept at 42° C. for 60 minutes. Next, this reaction mixture was kept at 70° C. for 10 minutes to stop the reverse transcription reaction, and cooled to room temperature. The resulting reaction product was subjected to gel filtration by using the Centri-sep™ spin Column (manufactured by Applied Biosystems). Each of Cy3-labeled cDNA and Cy5-labeled cDNA thus obtained was concentrated by ethanol precipitation so that they are paired for the same patient, and the precipitate was dissolved in 10 μl of a hybridization buffer (6×SSC/0.2% SDS/5× Denhardt's solution/0.1 mg/ml salmon sperm DNA) to give a fluorescence-labeled cDNA. Here, the 1×SSC composition is 0.15 M NaCl, 0.015 M sodium citrate, pH 7.0.

(3) Hybridization

A pre-hybridization buffer (6×SSC, 0.2% SDS, 5× Denhardt's solution, 1 mg/ml salmon sperm DNA) was added dropwise to the DNA array prepared in the above (1), a cover glass was placed thereon. Thereafter, and the array was kept at room temperature for 2 hours. Thereafter, the cover glass was removed therefrom. The array was washed with 2×SSC, and then with 0.2×SSC, and the washed array was air-dried.

Next, after thermal denaturation of the labeled cDNA prepared in the above (2), the entire amount was added dropwise to the DNA array prepared in the above (1). A cover glass was placed on the array, and thereafter the surroundings were sealed with a film. This array was kept at 65° C. for 16 hours. Subsequently, after the cover glass was removed, the array was washed twice in 0.2×SSC/0.1% SDS at 55° C. for 30 minutes, then washed once at 65° C. for 5 minutes, further washed in 0.05×SSC at room temperature for 5 minutes, and air-dried.

The air-dried array was applied to a microarray scanner [Affymtrix® 417™ array scanner, manufactured by Affymetrix] to analyze the fluorescence signal from each spot. The determined signals were analyzed with the expression data analysis software Imagene (manufactured by Biodiscovery, Inc.), and the expression level of each gene in the cancer tissue and the control normal tissue was evaluated for each of gastric cancer patients. An alteration in gene expression was judged by a relative expression ratio of each gene in the cancer tissue to that of the control normal tissue [intensity signal of expression in cancer tissue/intensity signal of expression in control normal tissue]. In this judgment, in order to make a correction for quality of the cancer tissue mRNA and control normal tissue mRNA to be compared, normalization was carried out by correcting an average of an expression level of the housekeeping genes. A relative expression ratio of the cancer tissue to that of the control normal tissue in each gastric cancer patient was calculated for each of genes of clone number 522 and clone number 584. The results are shown in Table 15.

TABLE 15

| Name of | | |
|---|---|---|
| Specimen | Clone 522 | Clone 584 |
| mk1 | 0.95 | 0.84 |
| mk109 | 0.51 | 1.29 |
| mk111 | 0.59 | 1.46 |
| mk115 | 0.45 | 1.77 |
| mk12 | 0.97 | 1.67 |
| mk13 | 0.5 | 0.68 |
| mk137 | 0.38 | 2.03 |
| mk14 | 0.34 | 1.3 |
| mk143 | 1.82 | 1.38 |
| mk144 | 0.37 | 2.4 |
| mk146 | 1.39 | 1.42 |
| mk149 | 0.28 | 0.84 |
| mk15 | 0.45 | 1.31 |
| mk153 | 0.91 | 1.42 |
| mk158 | 0.39 | 2.53 |
| mk16 | 1.12 | 1.29 |
| mk165 | 0.21 | 0.8 |
| mk168 | 1.07 | 1.58 |
| mk17 | 0.67 | 1.55 |
| mk170 | 0.3 | 0.95 |
| mk172 | 0.41 | 1.9 |
| mk173 | 0.37 | 1.85 |
| mk174 | 0.41 | 0.77 |
| mk175 | 0.6 | 1.04 |
| mk177 | 0.65 | 1.11 |
| mk18 | 0.2 | 1.69 |
| mk19 | 0.24 | 0.99 |
| mk2 | 1.02 | 1.28 |
| mk20 | 0.71 | 0.69 |
| mk21 | 1.36 | 0.81 |
| mk22 | 0.93 | 1.57 |
| mk27 | 0.41 | 2.38 |
| mk3 | 0.71 | 0.75 |
| mk30 | 0.89 | 1.89 |
| mk32 | 0.31 | 0.7 |
| mk33 | 0.4 | 0.85 |
| mk34 | 0.73 | 1.22 |
| mk36 | 0.51 | 2.06 |
| mk38 | 0.34 | 1.94 |
| mk41 | 0.52 | 2.23 |
| mk42 | 0.34 | 1.52 |
| mk44 | 0.78 | 1.43 |
| mk45 | 0.62 | 1.76 |
| mk46 | 0.38 | 2.08 |
| mk47 | 0.8 | 0.81 |
| mk48 | 0.26 | 0.92 |
| mk65 | 0.39 | 0.96 |
| mk75 | 0.4 | 3.51 |
| mk8 | 0.26 | 1.58 |
| mk80 | 0.25 | 1.81 |
| mk83 | 0.41 | 0.83 |
| mk92 | 0.95 | 1.82 |
| mk93 | 0.49 | 1.94 |
| mk98 | 1.06 | 1.48 |

In addition, Table 16 shows the number of patients in which a difference of two-folds or more is found in [intensity signal of expression in cancer tissue/intensity signal of expression in control normal tissue] for the genes shown in SEQ ID NOs: 1 to 65, namely the number of patients in which expression in the cancer tissue is enhanced two-folds or more that of the normal tissue. Also, the relative expression ratio in the specimens having the largest alteration of expression is together shown.

TABLE 16

| | Number of Patients | Maximum Value |
|---|---|---|
| | in Which Expression | for Relative |
| Clone | Is Enhanced in Two- | Expression |
| No. | Folds or More | Value |
| 20 | 6 | 3.3 |
| 95 | 11 | 10.03 |
| 132 | 6 | 4.89 |
| 291 | 4 | 4.79 |
| 337 | 4 | 5.38 |
| 344 | 1 | 2 |
| 354 | 2 | 2.54 |
| 377 | 3 | 3.05 |
| 386 | 1 | 3.37 |
| 394 | 3 | 2.66 |
| 461 | 4 | 2.81 |
| 503 | 3 | 3.09 |
| 507 | 1 | 2.27 |
| 556 | 1 | 5.82 |
| 561 | 6 | 2.84 |
| 584 | 9 | 3.55 |
| 589 | 3 | 2.74 |
| 629 | 7 | 4.27 |
| 778 | 1 | 2.31 |
| 801 | 11 | 10.27 |
| 877 | 1 | 2.46 |
| 882 | 5 | 4.55 |
| 883 | 8 | 5.9 |
| 944 | 7 | 12.02 |
| 1008 | 3 | 3.75 |
| 1014 | 1 | 2.04 |
| 1023 | 2 | 3.84 |
| 1028 | 8 | 6.11 |
| 1046 | 1 | 3.53 |
| 1043 | 6 | 3.35 |

Furthermore, Table 17 shows the number of patients in which a difference of two-folds or more is found in [expression intensity signal in cancer tissue/expression intensity signal in control normal tissue], namely the number of patients in which expression in the cancer tissue is reduced to one-half or less that of normal tissue, for the cancer-associated genes shown in SEQ ID NOs: 1 to 65. Also, the relative expression ratio in the specimen with the largest alteration in expression is together shown.

TABLE 17

| | Number of Patients | |
|---|---|---|
| | in Which Expression | Minimum Value |
| Clone | Is Reduced to | for Relative |
| No. | One-Half or Less | Expression Value |
| 42 | 6 | 0.23 |
| 49 | 2 | 0.38 |
| 68 | 10 | 0.33 |
| 129 | 17 | 0.02 |
| 155 | 11 | 0.34 |
| 203 | 2 | 0.27 |
| 295 | 8 | 0.19 |
| 323 | 12 | 0.36 |
| 382 | 5 | 0.42 |
| 383 | 13 | 0.32 |
| 426 | 2 | 0.21 |
| 430 | 7 | 0.36 |
| 449 | 6 | 0.38 |
| 511 | 8 | 0.38 |
| 522 | 29 | 0.2 |
| 552 | 16 | 0.26 |
| 609 | 10 | 0.32 |
| 645 | 10 | 0.29 |
| 668 | 11 | 0.19 |
| 731 | 8 | 0.37 |
| 817 | 11 | 0.34 |
| 818 | 5 | 0.18 |
| 845 | 6 | 0.35 |
| 854 | 3 | 0.4 |
| 925 | 6 | 0.24 |
| 1001 | 13 | 0.29 |
| 1018 | 11 | 0.29 |
| 1060 | 3 | 0.34 |
| 602 | 8 | 0.32 |
| 723 | 6 | 0.41 |
| 766 | 9 | 0.29 |
| 802 | 16 | 0.09 |
| 839 | 7 | 0.39 |
| 436 | 9 | 0.22 |
| 463 | 2 | 0.37 |

As shown in Tables 16 and 17, it can be seen that the cancer-associated genes shown in SEQ ID NOs: 1 to 65 alter their expression not only in gastric cancer specimens treated by the DD method but also in other gastric cancer specimens. Therefore, it can be seen that cancer cells can be detected by comparing the intracellular expression levels of the cancer-associated genes shown in SEQ ID NOs: 1 to 65 between the cancer tissue and the control normal tissue from a cancer patient.

### EXAMPLE 2

### Preparation of DNA Array

The 65 kinds of genes shown in SEQ ID NOs: 1 to 65 were selected from the genes of Example 1. These genes were searched with a computer for an about 300 bp region containing no repeat sequence such as polyA sequence or Alu sequence, and having high specificity to the desired gene, and DNA fragments thereof were prepared according to the method described in Example 1. Next, each of the above DNA fragments was spotted onto a support of the slide glass at a density of 33 dots/cm² to give a DNA array.

### EXAMPLE 3

### Preparation of Kit for Detecting Cancer

The 10 kinds of genes shown in SEQ ID NOs: 1 to 10 were selected from the genes of Example 1. These genes were searched for a 300 bp region showing a high specificity to the desired gene in the same manner as Example 2. Each of oligodeoxynucleotides having a nucleotide sequence corresponding to a strand complementary to mRNA of each of the above genes in this region was synthesized. Each of these oligodeoxynucleotides was labeled with biotin at a 5′-terminal thereof. Each of these 10 kinds of labeled oligodeoxynucleotides was dispensed to a separate container as a set of probes to give a probe kit for Northern hybridization.

Sequence Free Text

In SEQ ID NO: 3, n's at positions 234, 433, 459, 470, 513, 543 and 568 are a or c or g or t.

In SEQ ID NO: 4, n's at positions 455, 537, 543, 593, 620, 659 and 667 are a or c or g or t.

In SEQ ID NO: 7, n's at positions 609 and 663 are a or c or g or t.

In SEQ ID NO: 11, n's at positions 555, 580 and 667 are a or c or g or t.

In SEQ ID NO: 15, n at position 27 is a or c or g or t.

In SEQ ID NO: 17, n's at positions 121 and 238 are a or c or g or t.

In SEQ ID NO: 31, n's at positions 573, 580 and 591 are a or c or g or t.

In SEQ ID NO: 32, n's at positions 511, 529, 551 and 589 are a or c or g ort.

In SEQ ID NO: 34, n's at positions 553 and 597 are a or c or g or t.

In SEQ ID NO: 42, n's at positions 512 and 550 are a or c or g or t.

In SEQ ID NO: 60, n's at positions 547 and 583 are a or c or g or t.

In SEQ ID NO: 65, n's at positions 465 and 529 are a or c or g or t.

In SEQ ID NO: 82, n's at positions 165, 486, 503, 504, 512, 584, 599 and 609 are a or c or g or t.

In SEQ ID NO: 95, n at position 375 is a or c or g or t.

In SEQ ID NO: 97, n's at positions 663, 670, 679, 684, 688, 691, 692, 706, 714, 733, 734, 775 and 777 are a or c or g or t.

In SEQ ID NO: 99, n's at positions 663 and 678 are a or c or g or t.

In SEQ ID NO: 108, n at position 2 is a or c or g or t.

In SEQ ID NO: 111, n at position 859 is a or c or g or t.

In SEQ ID NO: 132, n at positions 651 and 674 are a or c or g or t.

In SEQ ID NO: 164, n at position 5 is a or c or g or t.

In SEQ ID NO: 176, n at position 4 is a or c or g or t.

In SEQ ID NO: 189, n at position 509 is a or c or g or t.

In SEQ ID NO: 196, n at position 3 is a or c or g or t.

In SEQ ID NO: 207, n at position 645 is a or c or g or t.

In SEQ ID NO: 208, n's at positions 6 and 648 are a or c or g or t.

In SEQ ID NO: 218, n's at positions 2 and 10 are a or c or g or t.

In SEQ ID NO: 227, n's at positions 687, 725, 734, 769 and 778 are a or c or g or t.

In SEQ ID NO: 249, n at position 415 is a or c or g or t.

In SEQ ID NO: 256, n at position 298 is a or c or g or t.

In SEQ ID NO: 261, n at position 285 is a or c or g or t.

### INDUSTRIAL APPLICABILITY

According to the method for detecting a cancer and the kit for detecting a cancer of the present invention, there is exhibited an excellent effect that a cancer, especially a gastric cancer can be simply and easily and rapidly detected. In addition, according to the cancer-associated gene, the cancer-associated protein encoded thereby and the antibody or a fragment thereof each capable of specifically binding to the protein of the present invention, there are exhibited some excellent effects that a cancer, especially a gastric cancer can be simply and easily and rapidly detected, and that suppression of proliferation of a cancer, especially a gastric cancer can be carried out. Also, according to the method for suppressing proliferation of a cancer cell of the present invention, suppression of proliferation of a cancer, especially a gastric cancer, is expected.

1. A method for detecting a cancer, characterized by evaluating alteration of expression of at least one kind of gene selected from the group consisting of:
(a) a gene comprising a nucleic acid having either a nucleotide sequence selected from the group consisting of SEQ ID NOs: 1 to 264 or a nucleotide sequence complementary thereto, wherein an expression level of the gene in a cancer cell is altered as compared to that in a normal cell;
(b) a gene comprising a nucleic acid capable of hybridizing to a nucleic acid having either a nucleotide sequence selected from the group consisting of SEQ ID NOs: 1 to 264 or a nucleotide sequence complementary thereto under stringent conditions, wherein an expression level of the gene in a cancer cell is altered as compared to that in a normal cell; and
(c) a gene of a nucleic acid having a nucleotide sequence having at least 80% sequence identity to either a nucleotide sequence selected from the group consisting of SEQ ID NOs: 1 to 264 or a nucleotide sequence complementary thereto, wherein an expression level of the gene in a cancer cell is altered as compared to that in a normal cell.

2. The method for detecting a cancer according to claim 1, wherein the method comprises evaluating alteration of expression of at least one kind of gene selected from the group consisting of:
H. sapiens mRNA for RING protein (GenBank accession number: Y07828);
Homo sapiens mRNA for PKU-β, complete cds (GenBank accession number: AB004885);
Homo sapiens carboxy terminus of Hsp70-interacting protein (CHIP) mRNA, complete cds (GenBank accession number: AF129085);
Homo sapiens mRNA; cDNA DKFZp58610523 (from clone DKFZp58610523 (GenBank accession number: AL050217);
H. sapiens Sp17 gene (GenBank accession number: Z48570);
Homo sapiens MD-1 mRNA, complete cds (GenBank accession number: AF057178);
H. sapiens mRNA for BiP protein (GenBank accession number: X87949);
Human chromosome 17q12-21 mRNA, clone pOV-2, partial cds (GenBank accession number: U18919);
Homo sapiens dead box, X isoform (DBX) mRNA, alternative transcript 2, complete cds (GenBank accession number: AF000982);
Human ferritin heavy chain mRNA, complete cds (GenBank accession number: M97164);
Human ovarian cancer downregulated myosin heavy chain homolog (Docl) mRNA, complete cds (GenBank accession number: U53445);
Homo sapiens full length insert cDNA clone YP42A04 mRNA (GenBank accession number: AF085884);
Human mRNA for protein disulfide isomerase-related protein P5, complete cds (GenBank accession number: D49489);
Homo sapiens Ku70-binding protein (KUB3) mRNA, partial cds (GenBank accession number: AF078164);
Human nonmuscle myosin heavy chain-B (MYH10) mRNA, partial cds (GenBank accession number: M69181);
Human voltage-dependent anion channel isoform 1 (VDAC) mRNA, complete cds (GenBank accession number: L06132);
Homo sapiens phosphatidylinositol 4-kinase mRNA, complete cds (GenBank accession number: L36151);
Human β adaptin mRNA, complete cds (GenBank accession number: M34175);
Homo sapiens H β 58 homolog mRNA, complete cds (GenBank accession number: AF054179);
Homo sapiens unknown mRNA, complete cds (GenBank accession number: AF047439);
Homo sapiens NRD convertase mRNA, complete cds (GenBank accession number: U64898);
H. sapiens mRNA for acylphosphatase, muscle type (MT) isoenzyme (GenBank accession number: X84195);
Homo sapiens transmembrane protein BR1 (BR1) mRNA (GenBank accession number: AF152462);
Homo sapiens dynactin subunit (p22) mRNA, complete cds (GenBank accession number: AF082513);
Human mRNA for aldose reductase (EC 1.1.1.2) mRNA (GenBank accession number: X15414);
Homo sapiens cDNA FLJ20693 fis, clone (GenBank accession number: AK000700);
Homo sapiens mRNA for scrapie responsive protein 1 (GenBank accession number: AJ224677);
Homo sapiens KIAA0402 mRNA, partial cds (GenBank accession number: AB007862);
Human transactivator protein (CREB) mRNA, complete cds (GenBank accession number: M27691);
Human MAL protein gene mRNA, complete cds (GenBank accession number: M15800);
Homo sapiens ataxin-2-like protein A 2LP (A2LG) mRNA, complete cds (GenBank accession number: AF034373);
Human phosphatidylinositol transferprotein mRNA, complete cds (GenBank accession number: M73704);
H. sapiens mRNA for transketolase (GenBank accession number: X67688);
Homo sapiens nibrin (NBS) mRNA, complete cds (GenBank accession number: AF051334);
Human SnRNP core protein Sm D3 mRNA, complete cds (GenBank accession number: U15009);
Homo sapiens FUS/TLS protein gene mRNA (GenBank accession number: AF071213);
Homo sapiens, clone 486790 diphosphoinositol polyphosphate phosphohydrolase mRNA, complete cds (GenBank accession number: AF062529);
Human globin gene mRNA (GenBank accession number: M69023);
H. sapiens mRNA for aminopeptidase P-like (GenBank accession number: X95762);
H. sapiens mRNA for UDP-GalNAc:polypeptide N-acetylgalactosaminyl transferase (GenBank accession number: X92689);
Homo sapiens cDNA FLJ20463 fis, clone mRNA (GenBank accession number: AK000470);
Novel gene mRNA similar to C. elegans hypothetical 55.2 KD protein F16A11.2, SW: P90838 (GenBank accession number: AL050255);
Homo sapiens cDNA FLJ10986 fis, clone mRNA (GenBank accession number: AK001848);
Human inhibitor of apoptosis protein 2 mRNA, complete cds (GenBank accession number: U45879);
Human MB-1 mRNA, complete cds (GenBank accession number: M80462);
Human tissue factor gene mRNA, complete cds. 1/1995 (GenBank accession number: J02846);
Homo sapiens mRNA; cDNA DKFZp566E0224 (from clone DKFZp566E0224) (GenBank accession number: AL050031);
Homo sapiens NADH-ubiquinone oxidoreductase subunit C1-B12 mRNA, complete cds (GenBank accession number: AF047183);
Homo sapiens ASH1 mRNA, complete cds. 5/2000 (GenBank accession number: AF257305);
Homo sapiens CGI-65 protein mRNA, complete cds (GenBank accession number: AF151823);
Homo sapiens, clone 24451 mRNA sequence (GenBank accession number: AF070599);
Homo sapiens ubiquitin hydrolyzing enzyme I (UBH1) mRNA, partial cds (GenBank accession number: AF022789);
Homo sapiens mRNA for cysteine-rich protein (GenBank accession number: AJ006591);
Human mRNA for KIAA0139 gene, complete cds (GenBank accession number: D50929);
Homo sapiens clone 23819 white protein homolog mRNA, partial cds (GenBank accession number: AF038175);
Homo sapiens connexin 26 (GJB2) mRNA, complete cds (GenBank accession number: M86849);
Homo sapiens mRNA for KIAA0914 protein, complete cds (GenBank accession number: AB020721);
Homo sapiens mRNA for KIAA0907 protein, complete cds (GenBank accession number: AB020714);
H. sapiens mRNA for transcriptional intermediary factor (GenBank accession number: 2X97674);
Human cytosolic aspartate aminotransferase mRNA, complete cds (GenBank accession number: M37400);
Homo sapiens CGI-118 protein mRNA, complete cds (GenBank accession number: AF151876);
Human guanylate binding protein isoform II (GBP-2) mRNA, complete cds (GenBank accession number: M55543);
Homo sapiens cDNA FLJ10633 fis, clone mRNA (GenBank accession number: AK001495);
Homo sapiens chaperonin containing t-complex polypeptide 1, eta subunit (Ccth) mRNA, complete cds (GenBank accession number: AF026292); and
Homo sapiens mRNA for Prer protein (GenBank accession number: AJ005579).

3. The method for detecting a cancer according to claim 1 or 2, wherein the alteration in expression of the gene is alteration in an amount of mRNA expressed of the gene.

4. The method for detecting a cancer according to claim 3, wherein the amount of mRNA is determined by a hybridization method or a nucleic acid amplification method.

5. The method for detecting a cancer according to claim 4, wherein an array in which nucleic acids or fragments thereof corresponding to the gene of which expression level is to be determined are each immobilized to a given region on a support is used in a hybridization method.

6. The method for detecting a cancer according to claim 4, wherein the amount of mRNA is determined by Northern hybridization method.

7. The method for detecting a cancer according to claim 4, wherein the nucleic acid amplification method is polymerase chain reaction or chain substitution reaction.

8. The method for detecting a cancer according to claim 1 or 2, wherein the alteration in expression of the gene is alteration in an expression level of a protein encoded by the gene.

9. The method for detecting a cancer according to claim 8, wherein the alteration of the expression level of a protein is determined by utilizing an antibody specifically binding to the protein.

10. A DNA array for detecting a cancer by the method as defined in claim 5, wherein the array comprises at least two kinds of nucleic acids selected from the group consisting of:
(A) a nucleic acid having either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell;
(B) a nucleic acid capable of hybridizing to a nucleic acid having either a nucleotide sequence selected from the group consisting of SEQ ID NOs: 1 to 264 or a nucleotide sequence complementary thereto under stringent conditions, wherein the nucleic acid comprises the sequence of a gene of which expression level in a cancer cell is altered as compared to that in a normal cell; and
(C) a nucleic acid having a nucleotide sequence having at least 80% sequence identity to either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell, or fragments thereof, each being immobilized to a given region of a support.

11. The DNA array according to claim 10, wherein the nucleic acids or fragments thereof are immobilized on a slide glass.

12. A kit for detecting a cancer by the method as defined in any one of claims 1 to 7, wherein the kit comprises at least one oligonucleotide consisting of a nucleic acid capable of hybridizing under stringent conditions to mRNA transcribed from a gene selected from the group consisting of:
(a) a gene comprising a nucleic acid having either a nucleotide sequence selected from the group consisting of SEQ ID NOs: 1 to 264 or a nucleotide sequence complementary thereto, wherein an expression level of the gene in a cancer cell is altered as compared to that in a normal cell;
(b) a gene comprising a nucleic acid capable of hybridizing to a nucleic acid having either a nucleotide sequence selected from the group consisting of SEQ ID NOs: 1 to 264 or a nucleotide sequence complementary thereto under stringent conditions, wherein an expression level of the gene in a cancer cell is altered as compared to that in a normal cell; and
(c) a gene of a nucleic acid having a nucleotide sequence having at least 80% sequence identity to either a nucleotide sequence selected from the group consisting of SEQ ID NOs: 1 to 264 or a nucleotide sequence complementary thereto, wherein an expression level of the gene in a cancer cell is altered as compared to that in a normal cell.

13. The kit according to claim 12, wherein the oligonucleotide is an oligonucleotide suitable as a probe for hybridization having at least 15 bases in length.

14. The kit according to claim 12, wherein the oligonucleotide is at least one pair of oligonucleotides suitable as primers each having 15 to 40 bases in length.

15. A kit for detecting a cancer according the method as defined in one claim selected from the group consisting of claims 1, 2, 8 and 9, wherein the kit comprises an antibody or a fragment thereof capable of binding to a protein expressed from a gene selected from the group consisting of:
(a) a gene comprising a nucleic acid having either a nucleotide sequence selected from the group consisting of SEQ ID NOs: 1 to 264 or a nucleotide sequence complementary thereto, wherein an expression level of the gene in a cancer cell is altered as compared to that in a normal cell;
(b) a gene comprising a nucleic acid capable of hybridizing to a nucleic acid having either a nucleotide sequence selected from the group consisting of SEQ ID NOs: 1 to 264 or a nucleotide sequence complementary thereto under stringent conditions, wherein an expression level of the gene in a cancer cell is altered as compared to that in a normal cell; and
(c) a gene of a nucleic acid having a nucleotide sequence having at least 80% sequence identity to either a nucleotide sequence selected from the group consisting of SEQ ID NOs: 1 to 264 or a nucleotide sequence complementary thereto, wherein an expression level of the gene in a cancer cell is altered as compared to that in a normal cell.

16. A nucleic acid comprising a sequence of a cancer-associated gene selected from the group consisting of:
(a′) a nucleic acid having either a nucleotide sequence selected from the group consisting of SEQ ID NOs: 66 to 264 or a nucleotide sequence complementary thereto, wherein the nucleic acid comprises the sequence of a gene of which expression level in a cancer cell is altered as compared to that in a normal cell;
(b′) a nucleic acid capable of hybridizing to a nucleic acid having either a nucleotide sequence selected from the group consisting of SEQ ID NOs: 66 to 264 or a nucleotide sequence complementary thereto under stringent conditions, wherein the nucleic acid comprises the sequence of a gene of which expression level in a cancer cell is altered as compared to that in a normal cell; and
(c′) a nucleic acid having a nucleotide sequence having at least 80% sequence identity to either a nucleotide sequence selected from the group consisting of SEQ ID NOs: 66 to 264 or a nucleotide sequence complementary thereto, wherein the nucleic acid comprises the sequence of a gene of which expression level in a cancer cell is altered as compared to that in a normal cell.

17. A method for suppressing proliferation of a cancer cell, characterized by suppressing proliferation of the cancer cell using a specific conjugate capable of specifically binding to:
(1) at least one nucleic acid selected from the group consisting of:
(A) a nucleic acid having either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell;
(B) a nucleic acid capable of hybridizing to a nucleic acid having either a nucleotide sequence selected from the group consisting of SEQ ID NOs: 1 to 264 or a nucleotide sequence complementary thereto under stringent conditions, wherein the nucleic acid comprises the sequence of a gene of which expression level in a cancer cell is altered as compared to that in a normal cell; and
(C) a nucleic acid having a nucleotide sequence having at least 80% sequence identity to either a nucleotide sequence selected from the group consisting of SEQ ID NOs: 1 to 264 or a nucleotide sequence complementary thereto, wherein the nucleic acid comprises the sequence of a gene of which expression level in a cancer cell is altered as compared to that in a normal cell; or
(2) a polypeptide encoded by the above (1).


SEQUENCE LISTING (264 sequences)

SEQ ID NO: 1 - DNA, 321 bp, Homo sapiens
gatcaagtcctagaggaggagaagaatttcctgctatcacggatttactggctgggtcatgagggaacggaagcggggaaacactatgttgcctccactgagccacagttgaacgatctcaagaagctcgttgattccctgaagaccaagcagaacatgccacccaggcagctgctggaggatatcaaagtcgtcttgtgcagaagtgaagagttccagtttctcaacccaacccctgttcctctggaactggagaaaaaactcagtgaagcaaaatcaagacacgactccatcacagggagcccaaaaaaaaaaaaaaaa

SEQ ID NO: 2 - DNA, 549 bp, Homo sapiens
gatctcagacggcaaatagatgaacaacaaaaactacttgaaaaatacaaagagcgattaaataagtgcatatcaatgagcaagaaacttcttattgaaaagagtacacaagaaaagctgtcaagcagagagaagagtatgcaagatcgattacgcctcgggcactttacaacagttagacatggcgcttcatttactgaacaatggacagatggttttgcatttcagaatcttgtgaagcaacaagaatgggtgaatcagcaaagggaagatattgaaaggcaaaggaaacttctagccaaacgcaaacctcccacagctaataattctcaggcaccctctaccaattctgaaccaaaacaaaggaaaaacaaagcagtcaatggagcagagaatgatccctttgttagaccaaatttaccacaactaatgatttggaagcctggaagaccagagttgcacaggaaccagggcaaccttgggtgatgaagcaacagaaactgctcatccttttgattgtgggatctccagtcgaaaaaaaaaaaaaaa

SEQ ID NO: 3 - DNA, 575 bp, Homo sapiens
  variation 234, 433, 459, 470, 513, 543, 568: n is a or c or g or t.
agccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatngaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaaacccccggnccctatacatagtttatgttcctggncaccccgacgnttccccaagttctgctgttggactctggactggtttccctttnagcatcggcttttgctgggccgtgatcgtncccctttgtgggcttggaaaagcangttaagg

SEQ ID NO: 4 - DNA, 675 bp, Homo sapiens
  variation 455, 537, 543, 593, 620, 659, 667: n is a or c or g or t.
caaacgtcggactcaggaagcctgcagtatatgttcaatgtcccctatggtgtgggcaaagctgcggtgaggacccagtggcataggagctgaggaggtaaaagacagtgatgttaccgatagctcaggaggtacaatccccagtggtcagaatgtgtgacaagctggctgctgactgtgcccacgagctgcggcgccatggggtcagctgtgtgtctctgtggccggggattgtgcagacagaactgctgaaggagcatatggcaaaggaggaggtcctgcaggatcctgtgtcgaagcagttcaaatcagccttctcatctgcggaaaccacagaattgagtggcaaatgtgtggtggctttggcaacagatcccaatatcctgagcctgagtggtaaggtgctgccatcctgtgaccttgctcgacgctatggccttcgggatgtggacggncgcccgtccaagactatttgtctttgagctctgttctctcacacgtgtccggcctgggctggctggcctcctacctgccctncttctncgtgtgcccaagtggattattgccctctacactagcaagttctaaccctnctggtctgacactacgtctctgcttgncttctcatttggacttggtggtttgtcctgtctcaatgnaacagcanccctttct

SEQ ID NO: 5 - DNA, 540 bp, Homo sapiens
caaacgtcggagacaaaagcagatgaaggttctgaagaaggagctgcgccacctgctgtcccagccactgtttacggagagccagaaaaccaagtatcccactcagtctggcaagccgcccctgcttgtgtctgccccaagtaagagcgagtctgctttgagctgtctctccaagcagaagaagaagaagacaaagaagccgaaggagccacagccggaacagccacagccaagtacaagtgcaaattaactgccctggtcaagtgtgtcagtgactgcacattggtttctgttctctggctatttgcaaaacctctcccacccttgtgtttcactccaccaccaaccccaggtaaaaaagtctccctctcttccactcacacccatagcgggagagacctcatgcagatttgcattgttttggagtaagaattcaatgcagcagcttaattttctgtattgcagtgtttataggcttcttgtgtgttaaacttgatttcataaattaaaaacaatggtcagagcccaaaaaaaaaaaaa

SEQ ID NO: 6 - DNA, 595 bp, Homo sapiens
ctttctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacaacaccaatcctacctccatcgctaaccccactaaaacactcaccaagacctcaacccctgacccccatgcctcaggatactcctcaatagccaccgctgtagtatatccaaagacaaccatcattcccccgaaaaaaaaaaaaaaaa

SEQ ID NO: 7 - DNA, 672 bp, Homo sapiens
  variation 609, 663: n is a or c or g or t.
ctgcttgatgtatgtccccttacacttggtattgaaactgtgggaggtgtcatgaccaaactgattccaaggaacacagtggtgcctaccaagaagtctcagatcttttctacagcttctgataatccaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctcaggaagacaaaaagctcaaggagcgcattgatactagaaataagttggaaagctatgcctattctctaaagaatcagattggagataaagaaaagctgggaggtaaactttcctctgaagataaggagaccatggaaaaagctgtagaagaaaagattgaatggctggaaagccccaagatgctgacattgaagacttcaaagctnagaagaaggaactggaagaaattggtcaaccaattatcaagcaaactctatggnagtgcaagc

SEQ ID NO: 8 - DNA, 524 bp, Homo sapiens
gatcgcattgtggagagggatggcctcagtgaagccgcggctcaaagccggctgcagagccagatgagcgggcagcagcttgtggaacagagccacgtggtgctcagcaccttgtgggagccgcatatcacccaacgccaggtggagaaagcctgggccctcttgcagaagcgcattcccaagactcatcaggccctcgactgaaaagttctcagtggggccagactggctcctggagctgacaagcgaccccgtggtgaggagaaatgggggccttgatgctcaccctggttcaggcccagaggtccaagctatactgtgcaggacatggccaggcctggtggacacaggaagcctacccaacacggtggtatttggccaacactgaggatgtggttcatgggggagcagtcccctccccactcttgctcatgggtgactcttacccacagctgactagggccagcgcaaatactggaacctgtaacagaattaaaggtgaatgttccgaaaaaaaaaaaaaa

SEQ ID NO: 9 - DNA, 284 bp, Homo sapiens
gttgcgatcctcaaacatgtggttatctttgttgtattggcataatcagtgacttgtacattcagcaatagcatttgagcaagttttatcagcaagcaatattttcagttaataaggtttcaaaaatcatgtaaggatttaaacttgctgaatgtaaagattgaacctcaagtcactgtagctttagtaattgcttattgtattagtttagatgctagcactgcatgtgctgtgcatattctgattttattaaaataaaaagttgaactgcaaaaaaaaaaaaa

SEQ ID NO: 10 - DNA, 504 bp, Homo sapiens
tacaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtacccgaaaaaaaaaaaaa

SEQ ID NO: 11 - DNA, 675 bp, Homo sapiens
  variation 555, 580, 667: n is a or c or g or t.
gatcaagtccaaaaccgatgtagaagaaaagatgtacagcgtaaccaaggagagagatgatttaaaaaacaaattgaaagcggaagaagagaaaggaaatgatctcctgtcaagagttaatatgttgaaaaataggcttcaatcattggaagcaattgagaaagatttcctaaaaaacaaattaaatcaagactctgggaaatccacaacagcattacaccaagaaaactataagattaaggagctctctcaagaagtggaaagactgaaactgaagctaaaggacttgaaagccattgaggatgacctcatgaaaacagaagatgaatatgagactctagaacgaaggtatgctaatgaacgagacaaagctcaatttttatctaaagagctagaacatgttaaaatggagcttgctaagtacaagttagcagaaaagacagagaccagccatgaacaatggcttttcaaaaggcttcaagaagaagaagctaagtcagggcacctctcaagagaagtggatgcattaaaagaggaaattcatgaatacatggnaactgaagacctaatatgtcacctncagggagaatcactcagtcctgcaaaaaaaactaaatcaaccaagaaaacaggaacagagatttaggaagagagattggaaaacctnacttaagg

SEQ ID NO: 12 - DNA, 522 bp, Homo sapiens
ctgcttgatgtctcctctggctttaaaaacccacatttgagtttcctccttccctctactgccctgtgcccatgcctgcccacctctcgttcttttgggcttctgacacctcccctcctccccactcctggcacctggctctgtgatacagaccgtctgcactgaatctgtgtgtaggtgtcccgcatgagatgtctgcagaatgagacttacagtgcgctgagccctggcaaaagtgaggacgttgtgcttcgatggagccaggagttcagcaccttgactctaggccagttcggatgagctggcgtctattctgcccacaccccagcccaacctgcccacgttctctattgttttgagaccccattgctttcaggctgccccttctgggtctgttactcggcccctactcacatttccttgggttggagcaacagtcccagagagggccatggtgggagctgcgccctccttaaaagatgactttacataaaatgttgatcttcgaaaaaaaaaaaaaaa

SEQ ID NO: 13 - DNA, 635 bp, Homo sapiens
ctgcttgatgacttagggaaagatgagttgtgagagccacaacagaggcttcagaccattttcttttcttgggagccagtggatttttccagcagtgaagggacattctctacactcagatgactctaccagtggccttttaaccaagaagtagtacttgattggtcatttgaaaacactgcaacagtgaacttttgcatctcaagaaaacattgaaaaattctatgaattgttgtagccggtgaattgagtcgtattctgtcacataatattttgaagaaaacttggctgtcgaaacatttttctctctgactgctgcttgaatgttcttggaggctgtttcttatgtatgggttttttttaatgtgatcccttcatttgaatattaatggctttttccattaaagaataaaatattttggacaatgccgataaatgtatgaagttagtatccacatcataaattcagagtgatgtttagcagtaaatcaatattttgaagtgatacacagatgtctttcctccccacaaacttttttaaacaaaaaacaagacctcttttctttagatggtgccacctatgcccaccacaacagagattttacatggaaaccgggctcagtgagaactgattt

SEQ ID NO: 14 - DNA, 634 bp, Homo sapiens
ctgcttgatgctatgaaacactcaggttgtgctgttaacaaagatagacacttttcttgcgaagactgtaatggaaatgtcagtggaggttttgatgcttcaacatctcagatagttttgtgccagaataatatccataatcaggcccatatgaacagagtggtcacacacgagcttattcatgcatttgatcattgtcgtgcccatgtcgactggttcaccaacatcagacatttggcgtgctcagaggttcgagctgctaaccttagtggagactgctcacttgtcaatgaaatattcaggttacattttggattaaaacaacaccaccagacttgtgtgcgagacagagccactctttctatcctggctgttaggaatatcagcaaagaagtagctaaaaaggctgttgatgaagtttttgaatcttgtttcaatgaccatgaaccttttggaaggatcccacataacaagacttatgcaagatatgctcatagagactttgaaaaccgtgatcggtattattcaaatatatgagcacaatgacatttttatattacagagcttccatcatcatgaagaaaaaaaaaatttggttctcaagtgaacaaacatataaaatgtaaacaatccc

SEQ ID NO: 15 - DNA, 576 bp, Homo sapiens
  variation 27: n is a or c or g or t.
ctgcttgatggcaaaaggggagggtgnagggtgggagaggaaatgtcagggcatgtgctctgataaaataaaggcagggaaacaagctgaattacttgaaattacttgaattttcttgtcttaaaactgaaaaaaaaatgtagttacacgttaaaatctgcaaatggtttttacacctctgattttaacatgaacttatactaatgtttggaatcttatgtcagaaatataagcagctatgtacttagaataggttttgaatgggagaggtagaacagagagagaattaagaagggatctgacttataaaagactagaatgtgattagagtgatagaacataccaatgttaccaagaaattgacaagctgctggctttaagcttatgcaagtggtagttgggaaagtaggaggtgtggaagagggtttgcattttggattaattcatgcaaaatgaaggaggaagcctggtctaagaagatactgtctttcaatagaaatgatttctaaactgctacagattaagaatagataatctgattgctgttgttttgtttgttcaaaaaaaaaaaaaaaa

SEQ ID NO: 16 - DNA, 484 bp, Homo sapiens
ctgcttgatggtgtgtacaaactcacctgaatgggacttttttagacagatcttcatgacctgttcccaccccagttcatcatcatctcttttacaccaaaaggtctgcagggtgtggtaactgtttcttttgtgccattttggggtggagaaggtggatgtgatgaagccaataattcaggacttattccttcttgtgttgtgttttttttttgcccttgcaccagagtatgaaatagcttccaggagctccagctataagcttggaagtgtctgtgtgattgtaatcacatggtgacaacactcagaatctaaattggacttctgttgtattctcaccactcaatttgttttttagcagtttaatgggtacattttagagtcttccattttgttggaattagatcctccccttcaaatgctgtaattaacaacacttaaaaaacttgaataaaatattgaaacctcgaaaaaaaaaaaaaaa

SEQ ID NO: 17 - DNA, 403 bp, Homo sapiens
  variation 121, 238: n is a or c or g or t.
gatccagtactatcagaatgacatttccctactgaggaggggaccttcgagggcctctgccccatgtgccctcaaagctgtcccacaatcatggagccctgcgacctccctgccctgccgncacatgcagtggaggagaggcctgtggcccaaagaacctggtagcgcctcctggggcagcacgtgggtggcgcagccttggtaacgccatggactgcagcgacaatcaatggatggngctgtctatgcacaggtgtgagtcctctgtttgcactggacatattccctacctgtcttatttcataggtacatgaagtattgtgtataaaaaaagagataagatttaaccaacatcaacaaaataaaaacccaaaatagtgctgtatcgaaaaaaaaaaaaaaa

SEQ ID NO: 18 - DNA, 395 bp, Homo sapiens
gatccagtacccttcaaccatgctgtgatcggtgcaagtcaagaactcttaactggaagaaattgtattgctgcgtagaatctgaacacactgaggccacctagcaaggtagtaactagtctaacctgtgctaacattagggcacaacctgttggatagttttagcttcctgtgaacatttgtaaccactgcttcagtcacctcccacctcttgccacctgctgctgctatctgtccttacttgtgggcttctccatgctgtgccaatggctggctttttctacaccctcttttgagtgtagtttggtattttgtaattgagagctcatttcaaaagcagaaaaagacaacaaatattaaagcaaggaaaagtgtaaccgaaaaaaaaaaaaaaa

SEQ ID NO: 19 - DNA, 351 bp, Homo sapiens
gatccggtaccacaacagaaacagaaacaatcgccaaatatgaaataatggatggtgcaccagtaaaaggtgaatcaattccaataaggctatttttagcaggatatgacccaactccaacaatgagagatgtgaacaaaaaattttcagtaaggtactttttgaatttagtgcttgttgatgaggaagaccggaggtacttcaaacagcaggagataattttatggagaaaagctcctgaaaaactgaggaaacagagaacaaactttcaccagcgatttgaatctccagaatcacaggcatctgccgaacagcctgaaatgtgaactgaacacgaaaaaaaaaaaaaaa

SEQ ID NO: 20 - DNA, 565 bp, Homo sapiens
gatccagtacgcaccacatttggagcaggagcctacaaatttgagagaatcatctctaagcaaaatgtcctatctgcaaatgagaaattcacaagcgcacaggaattttcttgaagatggagaaagtgatggctttttaagatgcctctctcttaactctgggtggattttaactacaactcttgtcctctcggtgatggtattgctttggatttgttgtgcaactgttgctacagctgtggagcagtatgttccctctgagaagctgagtatctatggtgacttggagtttatgaatgaacaaaagctaaacagatatccagcttcttctcttgtggttgttagatctaaaactgaagatcatgaagaagcagggcctctacctacaaaagtgaatcttgctcattctgaaatttaagcatttttcttttaaaagacaagtgtaatagacatctaaaattccactcctcatagagcttttaaaatggtttcattggatataggccttaagaaatcactataaaatgcaaataaagttactcaaatctgcgaaaaaaaaaaaaaa

SEQ ID NO: 21 - DNA, 345 bp, Homo sapiens
gatcgcattgcccacgagattgaagcactgaagtcattctcaaaatcagacctggtcaactggttcaaggcccatagagggccaggaagtaaaatgctcagcgttcatgttgttggatatgggaagtatgaactggaagaggatggtaccccttctagtgaggattcaaattcttcttgtgaagtgatgcagctgacctacctgccaacctctcctctgctggcagattgtatcatccccattactgatatcagggctttcacaacaacactcaaccttctcccctaccataaaatagtcaaataaataaactgcagtcacgttggcccgaaaaaaaaaaaaaaa

SEQ ID NO: 22 - DNA, 316 bp, Homo sapiens
gatcgcattgaccgcacaaacttttctaatgaaaaaaccatctctaagcttgaatactctaattttagtattagatactaatagaagagaaaaattgtaacacactgaacaatagatactgtatgttcttaagactatgtatactagaataatagtagcagagtagggtgaaaaggaactttctgttctgaaagctaagcgactgtacgtgctactaaaaatgtctgacactgaaataattttactcaactatgttttcaacaagcaaaaatatagtattctaagattaaaatgtcattacgaaaaaaaaaaaaaa

SEQ ID NO: 23 - DNA, 849 bp, Homo sapiens
gatcgcattgaaaacattgatcacctgggtttctttatttatcgactgtgtcatgacaaggaaacttacaaactgcaacgcagagaaactattaaaggtattcagaaacgtgaagccagcaattgtttcgcaattcggcattttgaaaacaaatttgccgtggaaactttaatttgttcttgaacagtcaagaaaaacattattgaggaaaattaatatcacagcataaccccaccctttacattttgtgcagtgattattttttaaagtcttctttcatgtaagtagcaaacagggctttactatcttttcatctcattaattcaattaaaaccattaccttaaaatttttttctttcgaagtgtggtgtcttttatatttgaattagtaactgtatgaagtcatagataatagtacatgtcaccttaggtagtaggaagaattacaatttctttaaatcatttatctggatttttatgttttattagcattttcaagaagacggattatctagagaataatcatatatatgcatacgtaaaaatggaccacagtgacttatttgtagttgttagttgccctgctacctagtttgttagtgcatttgagcacacattttaattttcctctaattaaaatgtgcagtattttcagtgtcaaatatatttaactatttagagaatgatttccacctttatgttttaatatcctaggcatctgctgtaataatattttagaaaatgtttggaatttaagaaataacttgtgttactaatttgtataacccatatctgtgcaatggaatataaatatcacaaagttgtttaacgaaaaaaaaaaaaaaaaaa

SEQ ID NO: 24 - DNA, 590 bp, Homo sapiens
gatcgcattgccatacctgatgcctctaagctgcaattcatcctagcagaggagcagtttatcctttcccaggttgcactcctggagcaggtgaatgccttggtgcccatgctggacagtgctcacatcaaagccgttcctgagcatgctgcccgcctgcagcgcttggcccagatccacattcagcagcaggaccagtgtgtggaaatcactgaggagtccaaggctctcctggaggaatacaacaagactacaatgcttctctccaagcaattcgtgcagtgggatgagctactttgccagctagaggccgccacgcaagtgaagccagcagaggagtgatagctgctccccatcccaaagtgggcctgggcagtcaggctccagggccctatgccaacctgcctttgttacaaggcagaggaagctttgtatttattggcttcaaggcccacctctctgtactctgggctctaaagttggaggtcaggttacctgaggtttgcaatttgcaacacccaccctccccccaatcagtgttcttattttagtgacaataaaccatagagatgaccgaaaaaaaaaaaaaa

SEQ ID NO: 25 - DNA, 536 bp, Homo sapiens
gatcgcattgctgagaactttaaggtctttgactttgaactgagcagccaggatatgaccaccttactcagctacaacaggaactggagggtctgtgccttgttgagctgtacctcccacaaggattaccccttccatgaagagttttgaagctgtggttgcctgctcgtccccaagtgacctatacctgtgtttcttgcctcatttttttccttgcaaatgtagtatggcctgtgtcactcagcagtgggacagcaacctgtagagtggccagcgagggcgtgtctagcttgatgttggatctcaagagccctgtcagtagagtagaagtctcttccagtttgctttgcccttctttctaccctgctggggaaagtacaacctgaatacccttttctgaccaaagagaagcaaaatctaccaggtcaaaatagtgccactaacggttgagttttgactgcttggaactggaatcctttcagcaagacttctctttgcctcaaataaaaagtgcttttgcgaaaaaaaaaaaaaaa

SEQ ID NO: 26 - DNA, 508 bp, Homo sapiens
gatcgcattgctggaaccaccaaggagtgtggaatgtccttgagtgtattatttatgcaagtcacagtcacgttgccatcatggcagctatgtgaaacactaataaatgtgtttttactttttattcccgttaaaactgatgtaaaacaggataaaggcttgttatagtcacttataagtatctgggtctaagtaatttccttagatgtttctaaagaaacattttcagctttgctcccattatgattccaataaggaacgctttcctagtgcaattttaggagtaaagtttgaagagataaaaatagccaaagataggagacgtctgaattttgaatgataaacagtgatgttttaaaaaagctgttgttcttcaggaggcatttgcctaggatattgctggattataccccattgggggcttttaattttatttgtatgaattttccaggatttcattaaaaattattattgtattttttaccttaaccgaaaaaaaaaaaaaaaa

SEQ ID NO: 27 - DNA, 444 bp, Homo sapiens
cttgattgccttagacaagtaagaatttatagaatctatgctctattgcactatatagaaactagaaatgcagttatactgatgtagtgcagtttgtgggaaatcaggaatggtgttctccagaatacatgaagattctcattgattgttgcaaggaatcacgaaagagatctttggtccaaagaaaacgtctctgaaagaaaatagagcatcaattaaattcaccttaaggctaagatattagttagtctattctaggttaaaaatagacagtgatttccaggatttcagttatgattattctttttaggtgactaaaatgtaaagagatgagtgggaggacttgccacagaagcaacaaagaccgagaaggtaatcgatttgtctactttccccgcatttgccattgatatcatgctatcattttacgaaaaaaaaaaaaaa

SEQ ID NO: 28 - DNA, 676 bp, Homo sapiens
cttgattgcccatttgggggtatttccttccaaagcagaacggaaaatcacatctcgtcctttcaccaggttccgcacggccgtcagggtggtcattgcaatattaagattacgttttttggttaggaaatggcaagaagtagatcggaaaggagctctggcacaaggcaaagcccctcgcccagggccccgagcacgacagccgcagtctccacccagaaccagagagtcccccccaacccgggatgtaccctctggccacaccagggaccctgccagaggccgcagactggcagcagcagcctccccacacagtgggggaagagccactccatccccaaattcaagattagaaagatccctgactgcttctcaagatccagaacattccttgacagagtatattcaccatttagaagtgatccagcaaagattgggaggggtactaccagattctacttcaaagaaatcctgccacccgatgattaaacagtgaataaaatgtcatggctctttcctgcgacaattctatttgaggaaaagatttgtttttcccttttcccaaggaagctcgtgggtcagcatgggcactactcttcatgtgcggtgacaccagcccccagatgccttgaattaaagtgtcctcaccttttatgcatgactgcaaagccagctt

SEQ ID NO: 29 - DNA, 558 bp, Homo sapiens
cttgattgccttagggacggaattaccccagcctcttgagctgaagtaacgtgtgggccgcatgcataaagtaagtaaggtgcaatgaagaagtgttggttgccaaattgacatgttgtcacattctcattgtgaattatgtaaagttgttaagagacataccctctaaaaaagaactttagcatggtattgaaggaattagaaatgaatttggagtgctttttatgtatgttgtcttcttcaatactgaaaatttgtccttggttcttaaaagcattctgtactaatacagctcttccatagggcagttgttgcttcttaattcagttctgtatgtgttcaacatttttgaatacattaaaagaagtaaccaactgaacgacaaagcatggtatttgaattttaaattaaagcaaagtaaataaaagtacagagcatattttagttagtactaaattcttagtaaaatgctgatcagtaaaccaatccctttgagttatataaccaagatttttaaataaatgttattgtcctcaccttcgaaaaaaaaaaaaaaaa

SEQ ID NO: 30 - DNA, 256 bp, Homo sapiens
aggtgaccgtgacctgagaaggaaagaaagatcctctgctgacccctggagcagctctcgagaactacctgttggtattgtccacaagctctcccgagcgccccatcttgtgccatgttttaagtcttcatggatgttctgcatgtcatggggactaaaactcacccaacagatctttccagaggtccatggtggaagacgataaccctgtgaaatactttataaaatgtcttaatgttcgaaaaaaaaaaaaaaa

SEQ ID NO: 31 - DNA, 600 bp, Homo sapiens
  variation 573, 580, 591: n is a or c or g or t.
aggtgaccgttacccagatgttgaagggattaaatacttccatgcctgaactggtgattggacttgttgaaatgtttttccttttttcctcttttgtcccctggcactgggatggtggtggtctgtgggtgctgtctcagggtgcccttagaaaaggacaactcagaagagtttcgtcagcgagagctgcgtgcggcccagttggctcgagagattgaatcaagcccccagtaccgcctacggatcgccatggagaacgacgatgggcgcactgaagaggagaagcacagtgcagtccagcggcagggctcagggcgggagagccccagcttggcatccagggaggggaagtatatccctctgcctcaacgagtccgggaaggtccccggggaggagttcgatgcagcagctctcggggcggtcggcctggccttagctctttgccacctcgtggccctcaccatctggacaacagcagccctggcccaggttctgaggcccgtggtatcaatggaggtgaagttatgaggtgactttgaggaagagggcaggggaaaggggatgccaaggangtggganggtaattggangggtttggg

SEQ ID NO: 32 - DNA, 643 bp, Homo sapiens
  variation 511, 529, 551, 589: n is a or c or g or t.
aggtgaccgtcaagttcaagtggtggggcctgcagaacaaagtggagaacttcatccataagcaagagaggcgtctgtttacaaacttccacaggcagctgttctgttggctcgataagtgggttgacctgaccatggacgacattcgaaggatggaagaagagacgaagagacagctggatgaaatgagacaaaaggacccagtgaaaggaatgacagcagatgactaaagccgcctttcccctctgcactttttgcaagacagtggtcaacaggaagacccagcagctccaccctcccaagtgacaggccattttcggacgcagccgttctgtgcccattcttcaggcaactttcgattccttactgtagctaattctagatgcccttcagtattgtgaacaaatagaccgtctggtgtcacagagcccgcgtgtgcaggagcctgctccttcgagatacgtggcgtgtaggttgctgtagttacagcgccttcgtctctcttcattgngttccgatccatttctgngtgttcccccaacctttatttncaagtgacattttcagtctttcaaatactggcttttgngatgtggtgatttaaatgatttaagttcattttggtctcaaaaaaaaaaaaaaa

SEQ ID NO: 33 - DNA, 480 bp, Homo sapiens
aggtgaccgttatcggggctggggtgaccctgcacgaggccttggccgctgccgaactgctgaagaaagaaaagatcaacatccgcgtgctggaccccttcaccatcaagcccctggacagaaaactcattctcgacagcgctcgtgccaccaagggcaggatcctcaccgtggaggaccattattatgaaggtggcattggtgaggctgtgtccagtgcagtagtgggcgagcctggcatcactgtcacccacctagcagttaaccgggtaccaagaagtgggaagccggctgagctgctgaagatgtttggtatcgacagggatgccattgcacaagctgtgaggggcctcatcaccaaggcctagggcgggtatgaagtgtggggcgggggtctatacattcctgagattctgggaaaggtgctcaaagatgtactgagaggaggggtaaatatatgtttcgaaaaaaaaaaaaaaa

SEQ ID NO: 34 - DNA, 622 bp, Homo sapiens
  variation 553, 597: n is a or c or g or t.
ttttggctccgggaacgtgtgttgttgatacaggaataacaaactcacagaccttaattcctgactgtcagaggaaatggattcagtcaataatggatatgctccaaaggcaaggtcttagacctattcctgaagcagaaattggattggcggtgattttcatgactacaaagaattactgtgatcctcagggccatcccagtacaggattaaagacaacaactccaggaccaagcctttcacaaggcgtgtcagttgatgaaaaactaatgccaagcgccccagtgaacactacaacatacgtagctgacacagaatcagagcaagcagatacatgggatttgagtgaaaggccaaaagaaatcaaagtctccaaaatggaacaaaaattcagaatgctttcacaagacgcgcccactgtaaaggagtcctgcaaaacaagctctaataataatagtatggtatcaaatactttggctaagatgagaatcccaaactatcagctttcaccaactaaattgccaagtataaataaaagtaaagatagggcttnttcagcagcagcagaccaacttccatcagaaactacttttcagnccgtctaccggaaaaaaaaaaaaaa

SEQ ID NO: 35 - DNA, 423 bp, Homo sapiens
gttttcgcagcggaccgaagagaagaaaagtaggccagagccgaactctcttcctgccaagatgtctattggtgtgccgattaaagtactgcgtgaggccgagggccacattgtgacatgtgagacgaacaccggtgaggtatatcgggggaagctcattgaagcagaggacaacatgaactgccagtggccgcaagaggaagaggacgtggaatgggacgtggaaacatctttcaaaagcgaagataattttctaagttgaacagaactttgtccttttttctttcaggttatctgagttcattggagtgggtgcttgtgcatatatgctaggtatcttttgccatctttctctttagatcaggggaaatgtttaagctaaataaatctggggggttttttgttcccaaaaaaaaaaaaaaa

SEQ ID NO: 36 - DNA, 286 bp, Homo sapiens
gttttcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcaaaaaaaaaaaaaaaa

SEQ ID NO: 37 - DNA, 271 bp, Homo sapiens
gttttcgcagcgagagcgaggaggaggtgctactcgtgagcagtagtcgccatccagacagatggattgtccctggaggaggcatggagcccgaggaggggccaagtgtggcagcagttcgtgaagtctgtgaggaggtatgcatggagaagagagggcacagctgtgcctcgtcatcatgaacctgtcctatctgtgtagttctttaatatgtcagcactcactgaataaatatacagtcacagaaaaagggcgaaaaaaaaaaaaaaaa

SEQ ID NO: 38 - DNA, 466 bp, Homo sapiens
gttttcgcagtggagaactcttggccaaaggtttttgcggggaggaggaggaaaccagctttctggttaaggttaacaccagatggtgcccctcattggtgtccttttaaaaaatatttactgtagtccaataagatagcagctgtacaaaatgactaaaatagattgtaggatcatatggcgtatatcttggttcatcttcaaaatcagagactgagctttgaaactagtggtttttaatcaaagttggctttataggaggagtataatgtatgcactactgttttaaaagaattagtgtgagtgtgtttttgtatgaatgagcccattcatggtaagtcttaagcttgttggaaataatgtacccatgtagactagcaaaatagtatgtagatgtgatctcagttgtaaatagaaaaatctaattcaataaactctgtatcagccccccaaaaaaaaaaaaaaa

SEQ ID NO: 39 - DNA, 630 bp, Homo sapiens
gttttcgcaggcaacaggcagactttgtggacctgagcttcccaacaatttccagtacgggacccaacggcgccatcattcactacgcgccagtccctgagacgaataggaccttgtccctggatgaggtgtaccttattgactcgggtgctcaatacaaggatggcaccacagatgtgacgcggacaatgcattttgggacccctacagcctacgagaaggaatgcttcacatatgtcctcaagggccacatagctgtgagtgcagccgttttcccgactggaaccaaaggtcaccttcttgactcctttgcccgttcagctttatgggattcaggcctagattacttgcacgggactggacatggtgttgggtcttttttgaatgtccatgagggtccttgcggcatcagttacaaaacattccctgatgagcccttggaggcaggcatgattgtcactgatgagcccgggtactatgaagatggggcttttggaattcgcattgagaatgttgtccttgtggttcctgtgaagaccaagtataattttaataaccggggaagcctgacctttgaacctctaacattggttccaattcaagaccaaaatgatagatgtggattctctt

SEQ ID NO: 40 - DNA, 666 bp, Homo sapiens
gttttcgcagcaaaagccctcatagccttccaaaaggcactcaggtgattgctagaaaccaagttcgccttgcagaagtctggatggatgaatacaaggaaatattttataggagaaatacagatgcagcaaaaattgttaaacaaaaagcatttggtgatctttcaaaaagatttgaaataaaacaccgccttcagtgtaaaaattttacatggtatctgaacaacatttatccagaggtgtatgtgccagaccttaatcctgttatatctggatacattaaaagcgttggtcagcctctatgtctggatgttggagaaaacaatcaaggaggcaaaccattaattatgtatacatgtcatggacttgggggaaaccagtactttgaatactctgctcaacatgaaattcggcacaacatccagaaggaattatgtcttcatgctgctcaaggtctcgttcagctgaaggcatgtacctacaaaggtcacaagacagttgtcactggagagcagatatgggagatccagaaggatcaacttctatacaatccattcttaaaaatgtgcctttcagcaaatggagagcatccaagtttagtgtcatgcaacccatcagatccactccaaaaatggatacttagccaaaatggattaagtgttcctt

SEQ ID NO: 41 - DNA, 662 bp, Homo sapiens
gttgcgatcccaatgaagaggagaaaaagctacaactatctaggtgaaatatctacttatcctagaagaagccttttgacagcaacataaaattcatacctataggtttttaattcctatggatgccgtagcaaataacaaaaacttggatggtgtaaagtaacagatgtttactcttttccagtattggagaccaggaatctgagatcaaggtgtgggcaggggtgcgctccccctggaggttctaggagaggatccttcctgcctcttccagctcctggtggctccaggtgtccctgggcttgtggccacatcactacattctctgcctccatctccacgtgaccttttcctctgtgtctgtgtctcctcttctgtctcttagaaggacactggtcattggatttaaaggccacctgggtaatttatagtgatctaatctcaagaatctttccttaattacatgcaaatactctttatccaaattagtttgcattcacaaattctggagcttagtacttggacatatattttggggggttgatggttggaggggcttttattcaactcagtacatcttaataaggaattaatgccccccaacttgccttacaagtcatatattaaaaacaatgttggcctggcgaaaaaaaaaaaaaaaa

SEQ ID NO: 42 - DNA, 564 bp, Homo sapiens
  variation 512, 550: n is a or c or g or t.
gttgcgatccgtgttgcctcacccaaactggttatggaagaggctcctgagtcctataagaatgtgacagatgtggtaaatacctgccatgatgctggaatcagcaagaaagccattaaactgagaccaattgctgtgatcaaaggatagaaccttggacagcagggctgcctgacaccaccaaccctctctgaagtggaagtggactgacatgctcttctgacatcagactcaaggcgggacaagttgcaaagtgtgcagctgtaactgctcacgccaaaatggctgatggggaggctgctgctttcaggggcccgtgcttgtaaaataaccttccaggaagaggcacattgcccacctttggaaagggaggaatatgccttctccttggttgttccacagagttttaggaaaatctgttagggatgggtagatgtcaaaccgccttacgcagtcatactgatctttagccatcagattgatcttcttcacaccaagctctgtttacattncgagaggtgtcatgaaagaaaggtctgttcaataaccnaaaaaaaaaaaaaa

SEQ ID NO: 43 - DNA, 522 bp, Homo sapiens
gatctgacactaaagggcatggtcaccggattgaaactgtctcaggaccttgatgatcttgccattctctacctggccacagttcaagccattgctttggggactcgcttcattatagaagccatggaggcagcagggcactcaatcagtactcttttcctatgtggaggcctcagcaagaatcccctttttgtgcaaatgcatgcggacattactggaagcaatggcaaaaatgagcaaagttgggaaagttgtgttcccgagactacaggataaaaaatactatgataagaaataccaagtattcctgaagctggttgaacaccagaaggagtatttggcgatcatgaatgatgactgaacagggcttgcaggtgctgatgccagaagcttctgtgccattgcattaaagacttctgtcatttgatccatgttcaagacccttgaggtattgtttcatcatttctgtattgtctttcaataaagaaaacaaacatgtgcaacccaaaaaaaaaaaaaaaa

SEQ ID NO: 44 - DNA, 375 bp, Homo sapiens
ctgatccatgggtagaacatgccaagtggtttccaaggtgtgagttcttgatacgaatgaaaggccaagagtttgttgatgagattcaaggtagatatcctcatcttcttgaacagctgttgtcaacttcagataccactggagaagaaaatgctgacccaccaattattcattttggacctggagaaagttcttcagaagatgctgtcatgatgaatacacctgtggttaaatctgccttggaaatgggctttaatagagacctggtgaaacaaacagttcaaagtaaaatcctgacaactggagagaactataaaacagttaatgatattgtgtcagcacttcttaatgctgaagacgaaaaaaaaaaaaaaa

SEQ ID NO: 45 - DNA, 504 bp, Homo sapiens
ctgatccatgtatgaggacatctcccggggcctccagggcacctaccaggatgtgggcagcctcaacataggagatgtccagctggagaagccgtgacacccctactcctgccaggctgcccccgcctgctgtgcacccagctccagtgtctcagctcacttccctgggacattctcctttcagcccttctgggggcttccttagtcatattcccccagtggggggtgggagggtaacctcactcttctccaggccaggcctccttggactcccctgggggtgtcccactcttcttccctctaaactgccccacctcctaacctaatccccccgccccgctgcctttcccaggctcccctcaccccagcgggtaatgagcccttaatcgctgcctctaggggagctgattgtagcagcctcgttagtgtcaccccctcctccctgatccgtcagggccacttagtgataataaattcttcccaactgcgaaaaaaaaaaaaaaa

SEQ ID NO: 46 - DNA, 376 bp, Homo sapiens
ctgatccatgccttacagggaaagtattctgagtactttcagtgaagaaaagagtcaggggatataaacgatggcttacgctgggtgtggtggctcacgcctgtagtccctgcactttgggaggcccagacaggcaaatcacttgaggtcaggagtttgggaccagcctggccaacatggtaaaagcccatctctactcaaaatacaaaaagtagctgggtgtggttgcacgtgtctgtagtcccagctactcaggaggttgaggcaggagaattgcttgaacctgggaggcggaggctgaagtgagctgagattggaccactgtactccagcctgggtgacagagcgagattccatctcaaaaaaaaaaaaaaaa

SEQ ID NO: 47 - DNA, 323 bp, Homo sapiens
ctgatccatgctgaggagaagcaaaatggcacagggctagttgccaccaaccaatttactgatgaatgaagcccaggggactgccattttataaatgccagcagttggaaaaatcgtcacgaattgacttagagcaagggtcagcaagcttgtctgtaaagggccaaacagtaaatattttagggctgggggccataaaatatgttgcaaccacccaattctgccattgtagtgcaaaagcagccatagacaacacatacatgaacgaacgtggctgtattccaataaaactttatttatggacacccaaaaaaaaaaaaaaa

SEQ ID NO: 48 - DNA, 322 bp, Homo sapiens
ctgatccatggggccgcaatgaagcttggagatacatgggtggctttgcaaagagtgtttccttttctgatgtattctttaaaggattcaaatggggatttgctgcatttgtggtagctgtaggagctgaatattacctggagtccctgaataaagataagaagcatcactgaagataatacctggaagcatcatagtggtttcttaactctccaaaataagatttcttctctgtagcctacttgtctggtttatcccttacagaatattagtaagatttaatcaattaaaatatatatatatgccgaaaaaaaaaaaaaaa

SEQ ID NO: 49 - DNA, 580 bp, Homo sapiens
tggattggtcaataaggactctgtgaaaaaactgggaactggcactacagcggtattcattaataaaaacttaggcaaaaagccaggaactatcactacagtaggactgctaagcaaagattcaggaaagaagctaggaattggtattgttccaggtttagtgcataaagagtctggcaagaagttaggacttggcactgtggttggactggttaataaagatttgggaaagaaattgggttctactgttggcctagtggccaaggactgtgcaaagaagattgtagcaagttcagcaatgggattggttaataaggacattggaaagaaactaatgagttgtcctttggcaggtctgatcagtaaagatgccataaaccttaaagccgaagcactactccccactcaggaaccgcttaaggcttcttgtagtacaaacatcaataatcaggaaagtcaggaactttctgaatccctgaaagatagtgccaccagcaaaacttttgaaaagaatgttgtacggcagaataaagaaagcatattggaaaagttctcagtacgaagaaaaaaaaaaaaaaaa

SEQ ID NO: 50 - DNA, 493 bp, Homo sapiens
tggattggtcccagttcaatactctgtatctccgtgtacgtggggatggtcggccttggatggtgaatatcaaggaggacacagatttcttccagaggacgaatcagatgtatagttacttcatgttcacccgcgggggaccctactggcaggaggtcaagattcctttttccaaatttttcttctctaatcgaggaagaatccgggatgttcagcatgagcttccgcttgataagatctcttctataggattcaccttggctggtaaagtggatggtccattcttcctggagatggattttattggcgtgtttactgatccagctcatacagaagaatttgcctatgaaaattctccagagcttaacccaaggctttttaaataaagatcatatggtagttttgttttactaatctaagggtactagcatctacaatgatatagacaaaataaaatatttctttaatggcatccgaaaaaaaaaaaaaaaaa

SEQ ID NO: 51 - DNA, 368 bp, Homo sapiens
tggattggtcacttagagccgactgtctcctctgccttttgtttttcagcttcagagacagatccaatatagtcccagggacctgggtctctgggagaggaaggaagagggagggagcaaagagattggggtatgtcccctgtagtacactcttacctcttacttcctagactttgatttctccggcagcccagatgttcagttctcttggcccctctctaccccttactgggatctggttttcattttccggtccttttgccatacacagttacagagatcagtcaaatccataccaccactgagatctcatttattgccacagatgcacaaaataaataacccaaaatcacgaaaaaaaaaaaaaa

SEQ ID NO: 52 - DNA, 271 bp, Homo sapiens
tggattggtccagaacagtttccggtcaatgagcactattttggattagtcaattttgggaatacctgctactgcaattcagttcttcaagcactttatttttgtcgtccatttcgggaaaaagttcttgcgtataagagtcaacctaggaaaaaggagagccttcttacatgcttagcagatctcttccatagcatagccactcagaagaaaaaggttggagtaataccccctaagaagttcatcacaagattacgaaaaaaaaaaaaaa

SEQ ID NO: 53 - DNA, 626 bp, Homo sapiens
tggattggtcagcactcatgtctccgttgtaaggcttgtttctgtgatgatcatacaaggagcaaagtgtttaagcaagaaaaaggaaaacagcctccttgtcctaaatgtgggcatgaaactcaggagactaaggaccttagcatgtcaacacgctccctgaaatttggcaggcagactggaggtgaagagggagatggagcttctgggtatgatgcttattggaagaacctttcatctgataagtatggtgataccagctaccacgatgaggaggaggatgagtatgaggcagaggatgatgaagaggaagaagatgaaggcagaaaggattcagatactgagtcatcagatttgtttactaatttgaatttaggaaggacctatgctagtggctatgctcactatgaggaacaagagaactaggggagctgctctggtggccgtgtgtgagaggagcaggagtgagtgtgtgtgcttgatgaattgtgtgtggttgttcaaaagtaccttagccacttagccttgcgcagaagactagttacacttaatgggccaagcaatagggtgtagcgtttttatagaactgataatcaggcttatggcattagaaaaatgagtttcaa

SEQ ID NO: 54 - DNA, 384 bp, Homo sapiens
tggattggtcctcatttgcaaagcatgccttcagagcagataagaaaccagctgacagccatgtcctcagtacttgcaaaagcacttgaagtcattaaaccagctcatatactgcaagagaaagaagaacagcatcagttggctgtcactgcataccttaaaaattcacgaaaagagcaccagcggatcctggctcgccgccagacaattgaggagagaaaagagcgccttgagagtctgaatattcagcgtgagaaagaagaattggaacagagggaagctgaactccagaaagtgcggaaggctgaggaagaggggctgcgccaggaagcaaaggagagagaaggagcgtatcttacaggaacatgaacaaaaaaaaaaaaa

SEQ ID NO: 55 - DNA, 334 bp, Homo sapiens
gatcaatcgcattcattttaagaaattatacctttttagtacttgctgaagaatgattcagggtaaatcacatactttgtttagagaggcgaggggtttaaccgagtcacccagctggtctcatacatagacagcacttgtgaaggattgaatgcaggttccaggtggagggaagacgtggacaccatctccactgagccatgcagacatttttaaaagctatacaaaaaattgtgagaagacattggccaactctttcaaagtctttctttttccacgtgcttcttattttaagcgaaatatattgtttgtttcttccgaaaaaaaaaaaaaa

SEQ ID NO: 56 - DNA, 396 bp, Homo sapiens
tcggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaattctgttgattaaagatgagctttgtctacttcgaaaaaaaaaaaaaaa

SEQ ID NO: 57 - DNA, 313 bp, Homo sapiens
tcggtcataggcagggaccaaaattccttgtcagctgaggaagtcctgaagaaacatcctgaagatgatgactgcactgccatcgtgggcagatgcagcttccatctacctgagggctgaaggggaaaacctttcacacacgtgaggaaggcgcagctctgtggaaaggtcactagaatggcagcggcagcaaatagggctccaatgcacgtttgcagttaactgggtccaaggagagcatggccctccacagcaagtttgctctatagaataaagtcctgagcttgtttttatcacgaaaaaaaaaaaaaaa

SEQ ID NO: 58 - DNA, 301 bp, Homo sapiens
gatctgactgctggctataggattttggacttaatgactgaaattgcaaattgtcctttttcttggcattacagattttgccaaaataactttttgtatcaaatattgatgtgtgaaagtgaaggagctagtctgctgaaccaggaatagtttgagatattgaactgtcatttttgcacatttgaatactttgcaggctggctttgtataaacttatcctctggtttcctatatgttgtaaatatttagaccataatttcattataaataaatctataaatattcgaaaaaaaaaaaaaaa

SEQ ID NO: 59 - DNA, 218 bp, Homo sapiens
gatctgactggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtggctattaaacaagagccggtgagccccgaaaaaaaaaaaaaaaa

SEQ ID NO: 60 - DNA, 675 bp, Homo sapiens
  variation 547, 583: n is a or c or g or t.
gatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccagtgaagaaacaccacccgtccagtaccaccaaagtagttctctgtcatgtgtgttccctgcctgcacaaacctacatgtacataccatggattagagacacttgcaggactgaaangctgctctggtgaggcaagcctctgtttaaaccggncccacatgaagagacattcctttgagacgaatttggagactgggattagagcctttggaggtcaaagcaaattaagattttatttaaggaat

SEQ ID NO: 61 - DNA, 560 bp, Homo sapiens
gatctgactgcatatgatatgaccctggcagagagttatgcccagtatgttcacaacctctgcaactctctctccattaaagtcgaggaaagttatgcaatgccaaccaaaaccatagaagtgttgcagttgcaggaccaaggcagcaaaatgctcctggactcagtgcttaccacccatgagcgagtggttcagatcagcggtttgagtgctacgtttgcagaaattttcttggaaataatccaaagcagtcttcctgaaggagtcagactgtcagtgaaggagcgcactgaagaagacttcaagggacgattcaaagctcgaccagaactggaagaactgttggccaagttgaagtagctactgtagaccctttcatgccagcagtggtcatattgagtgccaaagagaagagcttactgggtagttagagttcatcaggagaccaacccttagatttcataagtacccattcccatagccagtaatgtcctcactcctctgtggcttggctgtcttgccatttcttaccacttacctatgaggtaatgcttggtatc

SEQ ID NO: 62 - DNA, 551 bp, Homo sapiens
tcgatacaggccaaagaggtgctgaaaaaatatttggagtccaaggaggatgtggctgatgcacttctacagactgatcagtcactctcagaaaaggaaaaagcgattgaagtggaacgtataaaggctgaatctgcagaagctgcaaagaaaatgttggaggaaatacaaaagaagaatgaggagatgatggaacagaaagagaagagttatcaggaacatgtgaaacaattgactgagaagatggagagggacagggcccagttaatggcagagcaagagaagaccctcgctcttaaacttcaggaacaggaacgccttctcaaggagggattcgagaatgagagcaagagacttcaaaaagacatatgggatatccagatgagaagcaaatcattggagccaatatgtaacatactctaaaagtccaaggagcaaaatttgcctgtccagctccctctccccaagaaacaacatgaatgagcaacttcagagtgtcaaacaactgccattaaacttagctcataatcgaaagaaaaaaaaaaaaaaaa

SEQ ID NO: 63 - DNA, 616 bp, Homo sapiens
tcgatacaggccataattgaaatgggaataccttttaagtttcattaggggtggggtgggagtgggacaggaacaagacttgcctagatctttgttgtatcttggggacttttactttgttgtttgatgcttaaacttcaaaattctctgtattcaaatttgattgtggcgaatctacttcaaaaaggaaaaataatccaactttgtggatattaaatggaaggtttgctgttttgatctagttgtttccagtggagcagttttatgaaatatgttctataagatgtacattttttcattgtaacatagaaattgtaaataattgattaaagtgctgcattttgatgaattttttctagccatttttaaagagaaaactaggaattgagtattttgtgtacggtatgtttccatcctccctccccttcctcctcccctcctctctctctcttcctacctatttaattttcatttgtcatgaggtttttggatttgccaatgatctgctggacatcatgccccatgtcatagagaataaagctgatgattgtaccagtcttaaattattcatgattcaataaaattgatgcttatttattcaaaaaaaaaaaaaaaa

SEQ ID NO: 64 - DNA, 354 bp, Homo sapiens
tacaacgaggacattgctgacaactttgaagctttcgtgtgggagccagctatggtgcggatcaatgcgctgacagcagcctctgaggctgcgtgcctgatcgtgtctgtagatgaaaccatcaagaacccccgctcgactgtggatgctcccacagcagcaggccggggccgtggtcgtggccgcccccactgagaggcaccccacccatcacatggctggctggctgctgggtgcacttaccctccttggcttggttacttcattttacaaggaaggggtagtaattggcccactctcttcttactggaggctatttaaataaaatgtaagacttcgaaaaaaaaaaaaaaa

SEQ ID NO: 65 - DNA, 628 bp, Homo sapiens
  variation 465, 529: n is a or c or g or t.
gatcaagtccatcaatgaaaagtttgctgggtctgctggctgggaaggcacagaatcgctgaagaagccagaagacaaaaagcagctgggagatttctttggcatgtccaacagttatgcagagtgctacccagccacgatggatgacatggctgtggatagtgatgaggaggtggattatagcaaaatggaccagggtaacaagaaggggcccttaggccgttgggactttgatacccaggaagaatacagcgagtatatgaacaacaaagaagctttgcccaaggctgcattccagtatggtatcaaaatgtctgaagggcggaaaaccaggcgcttcaaggaaaccaatgacaaagcagagcttgatcgccagtggaagaagattagtgcactcattgagaagaggaagaagatggaagctgatggggttgaagtcaaaagaccaaaatactaatcactagntacaaccagagatgcttcacaaggatatgcttcccactggtttctttctacaatttccaaaggntgcaagacgtttttttgtggatgaatataaaaattttattggggtaattactttggttccattaaaattgggttaacttggcgaaaaaaaaaaaaaaaa

SEQ ID NO: 66 - DNA, 540 bp, Homo sapiens
gatcaagtccatacccagcagctatcaaggatttcaaggtctctccctacatctccctccacctactcctgttagaggtgttgaaattgtgctgtaaatgtacgtgcatggttatagctccaccatttagttcatgccattccctcagcccaaattcttctttccctcattctttgcctgttgaaagttttcttttcattcaaaaccagcagagcgcctattcctttctgaagtttttcctgatattcccaggcagagtcactcactcattccttcaaccatgtacctgttggtgtcacttgtacctcagtttggtgtgtttgtcagattgcatttctgcagacttagttgttgtcacatctcttactttcattagattgtaagcacattaaaggcaagggctgtattttcatcatatcttctagaagtactacctaacacaattgccagtacagagtagatgctcaataagttgctgattaaattaaatttcagaggaagtaatgtgataaacacctttaggtaaagcactgagacacg

SEQ ID NO: 67 - DNA, 536 bp, Homo sapiens
gatcaagtccaacaaaaacatgaaaagaaagacacacatggccaagtacattgtccttcccaggaaggtaagtaaggttggtttaacatccaaaaatgcaatgaatgtaactcaacatatttctaaactaaaaagaaaaaccacacaacaatctctaaagtggcagaaaaagcatttgacaaaatccagcatgtatacttgattaacactctcacagcctaatagaataaaacttcctcaagctgataaaggacatctacaaaaaccctacaattaacatgacacttaataatgaaggtctgaatgcatttcttctaagatcaagaacaagccaagagtatgttctcactactactaatcaatgttattctggagattctagccagggcagtaaggcaagaaaaagcaacaagtatagtaataatactgtcttgatgggcagatgacatgtttgtctatgaagaaaattcaatagaatctttaaagaaactcttgaaactaacaggtgagtttagcaaagctataccctatcaacg

SEQ ID NO: 68 - DNA, 511 bp, Homo sapiens
ggtatttttagtagagacggggtttcaccgtgtcagcgaggatggtctcaatcttctgacctcatgatccgcccacctcagcctcccaaagcactgggattacaggcatgagccactgcgtctggccgaaaaatagctgtttttttaaaaagccaacattatgctgaaacatggctaaatggttaacttacacttaaaattattcactggtgtccaataatacaagccaaaatttgtttatctagctgttagtcctctcctacttcttatagtcctgtctgctttgagtcggtccagtctatttggagaaatcagaccacaatgcttcaaaattactattacaacaacaaatcagatcacagaacagccaaaccccattcatatggtcaaaaagttacattttttcttcattaaacagaaaatagctcaactggtcaaaagaaaacatcagttctttggagatgaggattcctctccacatgtttagaaatagcacaccaggcaaatcaag

SEQ ID NO: 69 - DNA, 404 bp, Homo sapiens
cgagacagaatctcactctgtcacccaggctggagtcctgtgatgtgatctcgtctcactgctacctccacctcctgggttcaagcaaatctcatgcctcagcctcctgagaagctgggattgtagagatagggtttcaccatgttggccaggctggtctcgaactcctgagctcaggtgatccgcctgcctcggcctcccaaagcgctgggattacaggcgtgagccaccgcacccagctgcaagtttgattttaaacatgctaaatttgaggtgcttttaggacatccatgtgagggcatctgggaggcagctgacaatctggaggtctggagagtagtcagggttgaagacatcaacagaggtgatggctgaaatcttgggaggaggtaagggcaatcaag

SEQ ID NO: 70 - DNA, 157 bp, Homo sapiens
gtaaatgatagcatgatatcaatggcaaatgcggggaaagtagacaaatcgattaccttctcggtctttgttgcttctgtggcaagtcctcccactcatctctttacattttagtcacctaaaaagaataatcataactgaaatcctggcaatcaag

SEQ ID NO: 71 - DNA, 364 bp, Homo sapiens
cgagaaggagtctcactttgttgcccaggctggagtgcagcggtgcaatttcagctcactgcaacctccacctcccaggtttacgcaattcttgtgcctcagcctcctgagtaggtgggattacaggtgcccgccaccacgccctgcttatttttgtatttttagaagagatagggttttgccatgttagccaggctggtctcgaactcctgacctcaggtgatctgcctgcctcggccccccaaagtgctgggattataggagtaagccaccgcgcccggcccaataaagctctttttaagtgatcatgtctgaactctacagtaatgaggctttaccacattcccatgtgacccgacgtttg

SEQ ID NO: 72 - DNA, 625 bp, Homo sapiens
ctccacctcccagcctcaagcaatcctaccacctcagccctccgagtagctggggctacaggtgtgttgcacaccagcacgcccggctaatttttgtaccccctgtagagacggggttttgcttttgccatgttgcccaggctggtctagagctcctgggctccagcaatccacctgcctcgggctcccaaagtgctgggattacaggcatgagaaaccacactcagcctagtaccccattccttattattgttgaataatatttcattgtatgggcacaccatgtattttatctatccattcatcagttgatggacatttgggttgtttccactctggactattatgaataatgctcctatgaacatttatgtacaggtctttgtgtaacatatgtttccatttctcttgggcatatatctaggagtggaattcctgggtctttgcttaaccttttgagaacctgccaagatgttttccaaagcagctgtaccattttacattcccaccagaagtgtataaaggttccagttactccacatatattatgtctctctttctttctttcttttttgatacagagtcttgctctgtcacccagaatgaagacagtggggcaatcaag

SEQ ID NO: 73 - DNA, 457 bp, Homo sapiens
cgagatgaagtctcactctgttgcccaggctggagtgcagtggcactatctcagctcactgaaacctccacctcccaggttcaagcaattctcctgccttagcctcccaagtagctgggattacaggtgtgcgccaccatacacggctaatttttatattttgagtagagacagggttccgccatgttggccaggccggtttcaaactcctgacctcaagtgatctgccctcttcagcctctcaaagtactgggattataggcgtgacccaccacacccagcctctttttccttacttagagctaagaccaaacaatgcaaggaaacaaagtttaatattaatgagcaccgcccacatggcatcaaaccctgatgacagttccatgaagctatgattacaattcctattttataaatgaagaaatggaagctcaaggtcccttgctcacggtcacct

SEQ ID NO: 74 - DNA, 457 bp, Homo sapiens
aggtgaccgtacagttatcagatgataaattctaatcatgggacattcgcgtgggtcgtttgtaattgaagacagtgcatcctttgtaaccgaagctagtgcaaatgaggacaggagtggagggacagaaagcggagaggtgtacagctgctcttcttgcactgtacccctcatatccgcagccgggatcagtcaagatctcattcgtgatgcttgggtgccttgggggcttgggaagccagctcaggccttgtgatccactcggattagtgttgtctggggttctcactttctgactcctggaggacgaaacgccacttgtctgagagggagtgggagtgattcttgagaatgaaaacatgatttgggtagtgaagaggtggagaggagaggcagaggggatgtggaaaagaggtagaagcagggaagtaagcatgaataaagagtaaattgggcg

SEQ ID NO: 75 - DNA, 402 bp, Homo sapiens
cggcaagtcaaaccagagaaaatggtgaaacttttaagtgacagctgactcgagacagacggccctatagctgaggacccaggtaaggttagatgatcaatgtgtagcggcaccactctgcaacggtgcgacttctccagaagttgttcgaggaggaaagagaagtcattagagtagcccacagtggtgtctgctggggaaacgccgcagaccaacgcaaggatgggcgtaaacaaccccaagaagagctgggctgcctcctgtctctgcattcccatcacttctttccttcttggttttatccttaaccacgaagagcagttctcacccttaatgttgactcaacatttccttttgataataaatacttgcttagcgtctacttttactgcccgacgtttg

SEQ ID NO: 76 - DNA, 501 bp, Homo sapiens
cgtagagacggggtttcaccatgttagccaggatggtcttgatctcctgatctcatgatccacccgcctcggcctcccaaagtgctgggattacaggcgtgagccaccgcgcctggccgtgctgggccaagttttctatttttagtagagacagagtttcaccatgttggccaggcttgtctcgaactcttgacctcaagtgatcctccctccttggcctcccaaagtgctgggattgcaggtgtgagccaccatgcccagccagtatctatatatttcaaatgagtaagttaagggaatgaggaattgaggcaggagctagagagcaatggttcgtaatagtggatggctctggctgggtggagtatcttcatatgaatcccatgaaaatgctgaatattcccaagtacaacctacaggtgacagacaaagcctggcctttgtacacacgcgcaccactcatcacaaggtcccctcctacaagccccatgacggtcacct

SEQ ID NO: 77 - DNA, 502 bp, Homo sapiens
aggaacactgaatgaagtttattgaagagaaacacgtactgaaaacgttgaagagaagggaatcacaacacagagagaaactgcaaacggccaccacaggacacaggtcccacacaggtcccgggccccctcctcctggggttcctggcaagacgcctcatctgctcctactcctgctgcagcccccgctgccaactccctccttcctgagggccccggggtctgcctcttgccaggaaggtagactctggcagtgtccactggctttagggagccccctcccaagcccatctgctgggacagggccctgccctatggcccgcacaggacagtcacaggacactggaaaacacagcagggaacaccctaccaggtcaaggaaactgagtcatgagtaacaacaggggtggaacaccaaagcacaaagctcctcgggagcctcaggcagggcagccccgctccgtcctgggtccagttcttcctcccacagtcacggtcacct

SEQ ID NO: 78 - DNA, 393 bp, Homo sapiens
caaacgtcgggtaggaatgctgatcatgataggtttggttttctacagattctgttccggtgcctttcctatccaggcaccacctgagaaagttgtcatttgaggtcgcacttggaagttacatctgtgaagtttctgtcattcgtccagatctgtgtgtgtagcatgtgctgaggaagcacgtgctgggctgtgcctcagacagtgcatcaccgggcacccagaggcttgcctggctattcctgttctggtgtgtgtggagtgttggggaggaacagatgcagatcaacctgtggctgttttcccgtctaggttctcacaggtatctcctgacagaggtacttaacaatggctctgctggaaatttctataaataaaatgtccaaaatggcg

SEQ ID NO: 79 - DNA, 381 bp, Homo sapiens
gatcatggtcacaaacacgggtaaattctgtggattaaagaaagatatctagagatctacaaacacaaatctaaagaatccttgatttcactggaaatcaaagatgttgatgaaggtaatcatgagctattgtttttcactcataaaactggaggtgaaagacaaggattaacgtttctagcgatggtaaaagtgtaaaaagtatttggctgcgcggctggtggtgaataatttttacaaccttctcagaaaacaatatggaaacgtccagcacaagccagcagagaaagcctgtttgaggcctctgctgtggatagggatgcctgcctgaacacaggggaatcatgtgcatccatgctagctgcagtcattttggtaatc

SEQ ID NO: 80 - DNA, 266 bp, Homo sapiens
cggggggaagtgggaataattccaaaggaaatttaaaaaacaaatacatgaaatacaaaattgtgcagatagtacagtacagggacaaagatcccatagtgggagtcagattatctgcactcaaatctggttctgccacttactgactttggatgagcttcttaacctccctaggcctcaattttaagaattaagtgaaacaacccaagaaaaatttagcacagtactgagagacagaggcacacacacaagcacagaccatgatc

SEQ ID NO: 81 - DNA, 523 bp, Homo sapiens
gatccatggtcctttcttagaagcatttttgaacactaggaaggacattgagatgccctgtaacctttcagacaactacacataaaacatgctagatagttgataactcagtttgttaagcactgagtgttctctctgctccacacccagttctctagactctagggatacacagataaataagtatagctcttgttcttgtatgttatcagtgtgttaggaaatatttgaaaggacattttataaacctctagtttagcatttagtacccaaatgtattctctttttctgcacctagtagcaattgaaaatggttaatgcccattttctgagaaaggtccttgatattctcagaaaaattattgttgaattcttctttctgggttaagtaataccagctcattaaacatttcttcataaaattcctagctagatatggcaatttatagacctctctattccatgagaatgaacagacattgagacataattttatacatcagtatagaataggagtcacccg

SEQ ID NO: 82 - DNA, 611 bp, Homo sapiens
  variation 165, 486, 503, 504, 512, 584, 599, 609: n is a or c or g or t.
ttttggctccgtgggacgttgtaatgtgcacagacatttccaaggaaattctaaacagtcacccttcccttttgcattcccccaaatcttaagtgtatacataaaaccctgtgtacatattgttgtggtgataaaagggaattggttaaacagtacacttgggtnatggaactttctgtggccacctacgaaagacaagttaacaaactgtcatggagggctgttgttgcccagccagggccgctgcattttgacaacatttccaccctggccactcagcacatttcatggaggtcatgtcttttcactgatacttttttgatagtttttatataacaaaatccttattctatttataacttaagatgataaggcactataaattaatggcctaaaataatatatttgtctgttatcttttgctatttctacttcactttaatttttagctgtaaaattggtaaatggattcttacgactatctcnccccccccccccccccnnggtttttnaaatttaattaaatggcagaatttcctttttttaggaacaatgtgtattttcaattgcacctatttaaaaantaaactcaaaatttngcatgcccgnaa

SEQ ID NO: 83 - DNA, 480 bp, Homo sapiens
gttttcgcagcacagtcttatccagttccattattggggatgcaaagatgagtaaggaatgaaaacaggacaaaacctccgctcttgatgatcttcatttagccagagagaagtaagaaaagagttaacatcaatcatgaggtcctggaaggaagataagctctgaagtcaaatagacctgggatccaccagtaatgatggaaatttagaaggtaacttaaacctctcaggttcagtttcctcatctataggaagcgaatacaaatcattacaaatactagcaaactgaatccagcagcacaccataaaattaattcaccatgatcaaatgggctttattcctggggtgcaaatttggttcaacatactcaaatcagtaaatgtgattcaccacataagcagcattaaaaacaaaacagacatgatcatctcaatagacgtagaataagcttttgataaaatttaacatcatttcatgac

SEQ ID NO: 84 - DNA, 333 bp, Homo sapiens
cgagtaaaatagaaattctttaatatacagtgcagtttttataaagatgctctgtgtgttatgctgaacgcacaaacatccttagaaaatagtaatgtaaaaattcaaaagggtttccttattgcagatattcttggagatgttaatttatgcatctcctcgaagaggcacagccatccaaccatgttctcttttctttacaacagcttcaaacatttctcttctgagatgcatatttgggaaatgctggaataaattatatccagtctttcatatttgcatctatgcaaatctgctgaaaagcttaagtttattatttacagctgcgaaaac

SEQ ID NO: 85 - DNA, 243 bp, Homo sapiens
cgtaagagctggaggtgtttgggaagtgcctcagacacgctgttctctagccatggagtaccatctcctacctacttcccgtcacattccctgtgcaaaccccatctgttctctaaacctcgctcatgcagtctacccctcctcccagttcccatgtcgttgttttctcttccctacagccttaaccatttggctcctgaattgtaattctctgcccctgtgtcttctctgtcctgcgaaaac

SEQ ID NO: 86 - DNA, 857 bp, Homo sapiens
gggtgcagacagggttttaccatgttgcccaggctggccttgaactcctgagctcaaatgagtgctaggattatagggcatgagacactgtgtacagccatcattacataatattacatcagtcaattatgtaactaaaaccaaaaagattttaagatctcttttcacccccaggcaaaccaagaacactgataattgagtgaaggaggtcatttttaaggaattaggctccatagtgaattcacaaatattccaaaagacatcaccctcaggccaattttccaaaagtactatactatgctgcctttcaaatagcgtaatattaaaccgatgattccagtatgaatcctctagaactagaaccacattagtctaaataaaaacaatttagacaaagtccttggttgtccaacttgttccctcaagtcgttgggatctttactttctttctagtccttttctaattcagttgagtaaaaatcagtttgaaaaattaaccagtcagttactaactataaagttctgtaaatacttcctatcatcattcactgttttaatcacaccaacttgggaaatcaacacttgtaacagacacgctattccaaaaagcaagattcatttgggctccttgaaaagaactcaaaatgaagagaaagcattttcattaactcctaatttacacataagttaatcttttacagtatattaatgctattttgcatttttgagtagggatttggggatgtcgtcaaatagctccaaactcaaagtgcacctcagcagcagcagaggcatgagcctttacacaagtgagtcagagggaatccatgctcttgaatgtctagaaccgacgtttg

SEQ ID NO: 87 - DNA, 786 bp, Homo sapiens
ggggtcagaaatatgtcttattcacttttcaagacttttttcctgtgtttttaaaaaatgaattgaacggaattgattggcatcacctgaatgctgactaggacattgaagatcccatttcgtcactcatctttgtgagaaagacactaatacgataatgatataaccaccttcatttttgtatgcttagcagatactagaccatgcattcagcaccatcattagtattatctcatttatgatttttaacaactggagggtgtaggtactgttatcatccctacttaacacagattgaaaaacttccataagagcccaagctggtaagctgtggagctgagctagatttcaaattcagatctgtctgactccaagtcagtgttctgtcctataatgttctacagtcactctcaattagctggtgagacgttagaagctacaggtaaaaatggctagtccccaaagttctaccaactcaataagtgcacataattagtagggaattagttaggaataaaaggatgttcacagcctgggaatttcaatatctaaaacttccctaggcacctgacctttcctggctgtaggcattactcccaagaccagtaatccaggaacccagataatcaatcacccacagcacagcttccttgttgccatcaccatggcttttcagagcatctgtcaccatgctttgtcacacaaatagagagtgcagtcttcaactccgtgaaagtttatttctctgctctttgctaaaaacagggctctgtctactgcgaaaac

SEQ ID NO: 88 - DNA, 782 bp, Homo sapiens
cgtagaaatggggcctcactaggttgcccaggctggtctccaactcctggacttaagtgatcctcctgcctcagcttcctaaaatgttgggattacaggtgtgagttactatgcccagcctgaaaatataaagccacagattatccacaggctaattagtgaaaacagattgtggtatattgtgggacttagtatagaacaaggcttgattcataaaccagaggaaaatgagagactgattgttcaataaatgattgactatcaggtaaatagaatcagaagtgtatgttgacccctctttctataccaaaagtatactaatgatttcaaaagttaatgtaataaacaaataaagcctaaggaaaataataagtaaaattacatgtttatttatgttcagcaattgtaaggaccttgtgaacaaataaaagcaatggaaggcaaaaattaacaactttgacacagcatttttttatctgaatatcataaatcagaaacaaaattaaaagacaaactgtgaggaaaccgaataacaaatatggcaaagaattaaaaccctgaagaagtcatgtagatcaaagggtaacattaaaataaaagctcaatagggaaattgacaaaggggaggaacataatctatctaaaaagaaatacagataaccagcaaacaaatatactcagcctcacagagaaatgcaaattaggtgtttttttcctatcctttatttaaagatttttaaaagtgactactatacccagtttggtaagggggctgcgaaaac

SEQ ID NO: 89 - DNA, 539 bp, Homo sapiens
cgtgacggagtctcgcactgttgccctggctggagtgcaatggcgtggtctcggctcactgcaacctccgcctcccatgttcacgccattctcctgcctcagccttctgagtagctgggactacgggcgcccaccaccatgcctggctaatttttggtgtttttagtagagatggggtttcaccatgttagccaggatggtcttgatctcctgaccttgtgatccacccgcctcggcctcccaaagtgctgggattacaggtgtaagctaccacgcccagccacaaaaagcctgttttataataaggtgttgtacatctcatgtcatttattgaatactgtactatattatgtcaaaactgagacaggttcacaccattgtaaggtcaaaaagtcttaagttgaaccacccaaagtcaaggactgtctatatagcctattgttcctgtttctctgaagaaccctaactagcacagcgttatccactagattggatgaaagataatgtgtcttggacaaaggtggttgcttgcgaaaacc

SEQ ID NO: 90 - DNA, 534 bp, Homo sapiens
gttttcgcagtaacataccatgttagttcattttacaaatgagaaaatgggttatacaggaagtacataagttatgcagagtcacttacgagtggtagtaccgtgtcaagctctatggctctagcatctgtgcctaaactcctgtgttacatggcctatcaaataatgaggaaaggaaatttcaaaaattgtttgctctttaactggttggaaaaagaatcacaatgaatcacataaattttttctattggttgttagatatccttctgaatctccgtgaacaatctctgccccaaaagtagtgggcattcaattcagaaaagaaagataatttaaaaaaagataaaacaattacccttagttacacaattcattgttaacatgttatagttgttttaaggaacacatttatggctggacatggtagcgcatgcctgtaatcctggcagtttgggagaggctaaggtgggaggttcacttgaggcagatgtttgtgaccagcctaggaaatgtagtgagagtgctatctttacg

SEQ ID NO: 91 - DNA, 322 bp, Homo sapiens
gttttcgcagcagctgttctgtgtaggtgagagaatgtgtctttacaggtgggatttctgtttggatgtgtgggaaaggagaaggcatattgataagcagacaaacagacatatctatgcaaatacgcaactgcagaaataggcgaggagagctagtttattgcatgaaatcaataccgaagacttggacatccagtcaagaaaggagaggccaggcacgatggttcacacttgtaatcccagcactttgggaggctggggcaggtggattgcctgagctcaggattccaagaccagaacattgtaggaccctaactctacg

SEQ ID NO: 92 - DNA, 206 bp, Homo sapiens
cgctatcacattcaggctattcttaagcgtttttctgccatgatcacattgtgatgaagaacatgatggtcactagtaggtaactttctgtgtcattgccttactctcagtgaggtgctagtggatttacctacccctgcttttgcatcaccactgtaaatctaatagtgaaaaggcaaatgatgtctcagtatcactgcgaaaac

SEQ ID NO: 93 - DNA, 479 bp, Homo sapiens
cgaggtagagtcttactctgtcacccaaactggtgtgtggtggtaagatcacggctcactgcagcttcaatctcagcagactcaggtaatcctcccaccttagcctcccaagtagctgggtccagaggcccacaccaccacacccaactgattggtattttaggtagagacaggatttcaccatgttgcccaggctggtcttgaactcctgggctcaagtgatccacctgctttggcctcccaaagtgctaggattacacgcataagccactgtgcctggctgagattttatctacttgtaacctaacaaattagcatgacagtttcctgggtgctagcaggaaacctgagattcctgataagagaaaaaggacagtttatttctcccagtaatgatggtagccagactaccttcatttttgcatcagttccccagtaccaaaggcaaggtgaaatgggacacttgacactgcgaaaac

SEQ ID NO: 94 - DNA, 877 bp, Homo sapiens
cgacactggcagctcttctctttccctctgaatagagtcccaaaaatttgccaaagttgctaatctcgtttccctcttaaagggagcacgctacaattgaacccttctctccggcttttcagtgttttcctgatactttcacggacatccttaggcaaaataatttttctccctccaaattcagtcttgaaaacatcaaggcagaacatgcagctcttgccatttttagcatccatggtataataggccaaaggatttcccagctcaagtcaaagtgaaatttaaaactgcaacatcaaaatcatcttgagttactcctgtgcaaaacaaacaaaaataaaagtattgtagccaattttaacagcttagtaactattagaatatgtgtttttcacagtctacctgtgctagatgggatgaagggattataacgggggactttagggaatcctgcctctctattacatagaaaatgcagtatgaataaatactattcctgacataaagaccccttggaactaaccagggaaattcatcactaaatatgtgttcagcaccttctctgctgcaaaaggacagaaagagttaacttccctccagtgaaaaagaatcaattttcttttcctccagggatcaggcccactttacaataaactctttgtttaatgcaccacttacaaggatggactccacttaaagccacaaactcccgtaaggcatgtatgggtttatctctgtcctctatctgcctgcacttcactcacccatcatccagatcagttttgatctgttacatcctctcgtttgtcagcacttcaacccaatttggggggatccaattgtataatttcaagatgtgacctagggctgcgaaaac

SEQ ID NO: 95 - DNA, 876 bp, Homo sapiens
  variation 375: n is a or c or g or t.
cgatgtaaaaatgtggagagggagtacttggagagggggtgtattttcgttttgttttgttttgctttgtttgtagagatgggggtctcatgttggccaggctggtctcaaactcctggcctcaagcaatccttccagcttggccttccaaagtgctgggattacaggcgtgagccaccatgccaagccaagaattttaaaaaattagccaggtgtgttggtgcacacctgcagtcccagctcctggcttggctgaggcaggagtgtcgcatgaacccaggagttcaaggttacggtgagctatgatcgcatgtgtactccagcctgggagcctgggtgacacagttaagaccctgtctcttaaaaaaagaaaanaattaaataaatgctgatggaaaatctaaatagatctggacctatcttccttaggcactcatcacatctctatttttaatttctccaaaggattatttatggtcttatcttggcctgaggatactaagtggaagttaacataatgctgtttgaggaaaaataaaaataaacaggcacttacaggaatctgttaccaacagtatctaaacaactgaagccaagtgttttctggaatcatatctaagatttgctgaattttctactgtgactgaggagctgcattcttgtgaccaaaaaaagctattaaaaaagaaaagaaaaccagacacgtacacacaaacttgggggcagacttgttggcaatatcgatgaaaatgacctacaaaaagaacttggactcacacaaggaccataggagagtgggaagaaggaaaccacgcaccgctctgaagcaacataaattgcaaaaggaaacaactggagatttaacactgcgaaaac

SEQ ID NO: 96 - DNA, 802 bp, Homo sapiens
gttttcgcagcagtagagagggacagcctggggaacgctggggccctgctgaatcccgggctctccaggcacgaacaggggcatcccgcgtccacgccgcggggaggagggtttcaccgtctccagggacttggctcgaggaaattaaactctaatgagtagctcgagaaatgttccgggatggtggatgagcgatcatccttaaaagaaaatgctattctgggagctccaacctgcaattaacctacagaaggaaccttttgagaggctggtgcagcgcttcggggaggcagattaagaacttagttggcctggttgaagctctgtgaggagcaaagcagccctctccaggtgaactgcttgactttaccacctgaaggagtatttactgcaagaattaacaaagcaggtgttcctgatccaagcagaaccctgttatttgaagagcctgagaaatcttatcccgtttacaaatgtcctgggagtgcatccaaagtgaaacttgggtggatgaaccgcatcaaagacttccagaagccactccagtgaccataagataaatgtcagaatcgcataacccccaggatcagagatggagtctcgctctgtcacccagcctggaatgtagtggcatcattgtaactcactgcagcctgaaactccttggcttaagtgatcctcccacctcagcctcccaagtagctggaactataggcacaagctaccatgcctggctattctttttcgttaattttttttaaactaaggaatgaagaaaataaaacattacatgactggtacg

SEQ ID NO: 97 - DNA, 783 bp, Homo sapiens
  variation 663, 670, 679, 684, 688, 691, 692, 706, 714, 733,734, 775, 777: n is a or c or g or t.
cgtagaaacaaggtcttactatgttgcccagggtggtctccaactcctgggctcaagcaatcctcctgccttggcctctcaaagtgccgggattacaggtgtgagacactgcacctggccccttattttgtaactcttatcaccattataacaacaccgatggtatctttttaatgtcgtcagtctgccctactaaactgcaagctgtgtggaggcaagaaccatgtctgttcactgctgtatttctagcaccaagtggagcgtctggagcacaggtgagagtaaccattataaacggtcctcaccattgtcccagccatccagaataaccagctaacttcacagagttaagaggtaaacactttcagatcagttgggttcacagatgtgcaatattgtcagatgaaaagtaaattcaacatcttcctggtttccaaaggcagaatcaagaaaaacctgaaaaacaactgcggaccctctgaatttaaactgacagctaacttcagattcaaaatttgggaatcacgcctataaaaccaagcaaactagtagattgactctggtatgaaacaccatcatgggtaacagccctgcctcccatctactcaatctggacaaaaagagggaaacaggcaaatgtgtgtgtcttctgcttgcccttanttttcanccttccacntttantggnacnnccgaaaaccctggngtgaaatngtaacccggtccaattggnngaccctcaaggcatgcaagcttggcactggccgtcgtaatngncatcaa

SEQ ID NO: 98 - DNA, 272 bp, Homo sapiens
cgtaaatgcctgatttgtaactgttgatgtctacttttaatgaaacaaacaacatgtttaatgacaattacacttccaaagtgaggagaaacggcaggcagctgccacctaaaaatcagccctgattcttttctcaggtctcaggctggctttcctctctgataactgcaatgctggtctttggaggggacaaaaggagagcccacagggtgggaaaccgtctccccatattgctgtggagtatgtgtgtctttaatttcctggacttgatc

SEQ ID NO: 99 - DNA, 684 bp, Homo sapiens
  variation 663, 678: n is a or c or g or t.
acaagggttttcgcagatgagaagatgagaaaggtcactagcacgtgtggcctgccacacagcaggcacccagtgagggtggactcaacttccatgaagcataagaggcatccgtctgtcccaggcctttgggtgggcacttgggccttttggaagtgcctgatgggtttagggaatttgtcttctctggaaatcagctgacggctcatgaatttgtgatattttaaagtctctggctgggcattttggctcatgcctgtaatcccagtgctttgggaggtggaggagggaggattgcttgaacccaggagttcaagaccagcctgggcaacagtgagatcgcgtctctacaaaaatttttttaaaaaattagctgggcatcagccaggcgcggtgtctcacgcctgtaatcccagcactttgggaggccgaggcaggcggatcacgaggtcaggagatcgagaccatcctggctagcacggtgaaaacatgtctctactaaaaatacaaaaattagctgggcatggtggtgggcacctgtagtcccagctgctcgggaggctgaggcgggagaatggtgtgaacccgggaggcagagctggcagtgagccgagatcgcgccactgcactccagcctgggcgacagagcgagactccattcgnaaaaaggatcaccgngtaccg

SEQ ID NO: 100 - DNA, 536 bp, Homo sapiens
gttttcgcagctactaggtaaaactgatcttaaggttatagcaggtcatttcagcagctggcttgccagataatccctgggcaggtgcttgtgccctaagtgcttttcttttccttttcttcctccctcatctcatgactgtaatttaattgggtatgacaagataattccaatttatataatcaattttcacaatatccaatgcatccaaaattaatttttctttatatttttatgggttacttgagttccatgtacttgttataatgtattgaagtttataattaatctttcgaatgtaaacttaggataattagaatgttgaagatatgatgactatattatagaaatactcattgggctatgcagttaatatgcttcagacgtggtatgtaaatgttgatggaaacaatatttcatgtcggttcagattcagtgttttattttttgaaacattgcagtggaatttttatgtttaattctgttagctggtaacagaacttctggggcaattttgtttcagtttgttgtaactc

SEQ ID NO: 101 - DNA, 619 bp, Homo sapiens
ggtagggaatggtgtcttactgttgcccagcatggtcacgaattcctggcttcaagtgatcctcctacctcagcctccaccctgcctggggtctccctgagggctgggactgagatctgttgtgtgtactgccatattcccaaaacctaaacgatgccttgcatggaacaggggatcagtggtgatttgttgaaggaaagactcctgtctgtccccactgtcctggcctccagacccccctacctgtgaggggtacaggaccctctggggaggggccgctccaacagctggcttcgttggtaacagtggctgtaggggtcttgtcaggttctgggtcttctgagagtagagggtctccagccagcatctcaagggtcctgggggataaaggtgtgtcaggatgccaccttacacccagtctgtcctgccaaggggtgctggggaccttagggttgtttgtggcaaacatcacctctgtggcatctcttgtgtctgggaggtctgaagaatggtgttttgggggaatcaggtgagttttttttaaataggacttgaaaaagcatctgaagggccccagataactttgaggttcatagagtgggattatagggatcgcaac

SEQ ID NO: 102 - DNA, 788 bp, Homo sapiens
cgatatcagtcatgtggggattctcagtcattctttagatcaggctagatttaggacacaacattcagtgatagctcttgacctgagctgctgatgaccactgccctggtctcgcaagaggatcaatcttacatgtggatgacagtgcacaatatgatgggtaaaccaaggtatcatgaaaaatatggggctcttcttccaaaccaggttgttcatgctgatgacatcaacatacccaaatcatgatttgctggttacaaataaagcattgcaaaaaccacactggcctggaatagcagggatcatcacaaaactaagaaaagttctggttattcttccacaaactcagcgcaacattcgattggtagggcacggtgggggaccaggaagagatagagatttagccaagatatgccccaaagccactgcttttctggactcaaaccaggggattagggaaacaacattccccagcttctatgcaaagagccaaagggaacacagacttaaatatcatgacatgaaagttacttcacttgatgaaattacaaaatcattggtaattgctgagaatctctcacctctagggttttacaatgcaaaaccccaccactagagttaatgcgctatagtcaatctctcttctatgagggagggttgtcggggagtgggatatggtattgcctgggaatccctaaagaagacagagaggcacaaaatgatggagaagggcaccagagaaacaatctcctcaaactacaattactttattctttggccgacgtttg

SEQ ID NO: 103 - DNA, 481 bp, Homo sapiens
gggctcactcatggttagctattttcttatgctcactattttcttatgctcaattcacaaacttgctttttatctggttcagatggcagatagacatgcgtggagaataaataacatgtcatggaaagagcccttgagttggaataagaaaaccttgattaaaatccaagctcttaataacattattaatgacatgaggggtatgtgttctttaacctcgctctctgagtctgtttcctaagatgcaataattacattagctaatgcatttaaaaaggtgatcactatgtacaagttatatattatgattagtttgaaagaatggggtatatcttatctctaactttaatcctaagcttcagaaatagaattgttcatcagacaagaacataagggggcaactgggacacacaataggttcacattggcctcagtgagctacgaatatagaggtaggcaaacagcagagacggatcgcaac

SEQ ID NO: 104 - DNA, 192 bp, Homo sapiens
gttgttgttacaagaagcatgaggacttagattttcattttataaattttttaatcattggcattaagtagacctgcgaaaatgagaacgaatatatattaagtaaaattaagttcaaaagttagtagtaaacaccatgtgacagaagctgggccagtatatatgtaaatttcacgtgtttaggatcgcaac

SEQ ID NO: 105 - DNA, 821 bp, Homo sapiens
cggacaaagatgatttattttcatacctaaagcactttataatattctctggaaaatctaaaatcattctgttatcctaacatttttatactatcatcattttagaaaataaaaggcctgcgttatatactagaaaaatttcttcattatatgcaaaatatttatctcctctagtaaaggagattaaagaacaactgcaagaggaaggaaggtcctgaaagtgtttcatttggtatctacctaccccaaccccaagacataaagacagataaaggcactaagatgctagtatgtggctagtcctttcaataacccagtcagtccatacagataacccatgggatatattcaagccactctttgagccatcgatggtcattatttggttagttcacccaaggtaaggccataccagctgttaaaatgatgtagagattaatcaacagggctgccacttgcgaatcccctccaaggatgctgtgcaaagggtctcattggtcctgatgagtaatcttgtgactgtacatattcctgggtgcatgtccacaaatactgaggtatagcctgcatgccactaaaaataacaaaggtttcagggctggagacattgtcaaccacactgtcatgaccatctatagctccaggactcagtggtgggggaacaatatttaacggatgtccctggcagaagaagcctgtgagtacttcagcctcaaacacatagatcagcttatctgcagcagagattttcttggccttctctgccaggtttttgaggttcttggtgaagtatatgccagctccgtattttggatcgcaac

SEQ ID NO: 106 - DNA, 669 bp, Homo sapiens
gttgcgatcccctttgacagttttgtaatgaaggcaggaagtgaatttgagcaaggcccccacgtagccatgtgacccgagcaaggcagcacccgccttggagcccctttcccgcctgcgagggagacctggcgtctgccccgctgctcgcagagtcacgttgccggcggcatcaaagggcgaggaaaggatgaggaagcgctttgtggtctgaggcagcccctctgtcatgtggtgctccctattcctcctcctcgtcttcctcgtcttccccctcctcctcgcgcccggcaggcaccctcgctactcaggagttctgtccatcctcgttttgtgtggctgactcagttctcgagcccccttcctcccatgcagaaaggggacagtgtggcaacccgcccccaacctctattcttggtgaatttaaaatgggggttcccagagtgggtgctggcaccccaggggacaccacacagaccaccaggctgcaggagggagacacaggccgtgctgtctgtgttatggagtgcaccctttaaaagttccgaggtggtctagccagggggcggtggctcacgcctgtaatcccagcactttgggaggccgaggcgagttcaggagatcgagactattctggcttggctagcgtggtgaaaccccgtctctacg

SEQ ID NO: 107 - DNA, 398 bp, Homo sapiens
gatctgactgctgttgggatgagagactgaaatagaagagtcaaagttgacctggaagttataaccctgtttaaccaggagagttaacattgacattgatagaagtaagggtgaacttagatggggagcccattcataattgatttttgttttttcacttgcatgcatagtagtcaaacaatatagaaaagtattcagtgaaagtctctctctcgatctatctctcatctatgcccagtcccctccttggagaggatcactgttaccagtgtctgctacatccttcctgagaggctgtaagctaggggtcggccaactgtgacccatgggccacatccagcccactacctaattttgcgtgacccttaagctaagaatggggtttatatttggtgccc

SEQ ID NO: 108 - DNA, 424 bp, Homo sapiens
  variation 2: n is a or c or g or t.
cnaatagaccttgtgcattctactgctgcccacgctgctccaactcagtgcccactggaacctaaatgaaatcccacatcctaaccacgtgcaactggcctttgccatctctctcaccccaacttcttccaatccccattttattcattatctccagccatgctggactttcttctacccctttcttctatgttgaatttatctctctcaggcctttgcattgttcttctttctgcgtggaatgttttgaagatcctggctaaaagcagtacctctcgcacaaatcattctgccatgttactcgagtttatctttgtcacagccctatgagagcttatttgtgtttactgtctatttatccccattagaatataagttccaaggggttatcttactgactgctgtacccccgtgcctgtatcga

SEQ ID NO: 109 - DNA, 788 bp, Homo sapiens
gttctaggttaatctgagcaatctcttcacttttgtctgtgatgtattttagcagtgaattactcctcctggctaagagcttcacatcttcagtgttaattgtggttcttttcgcatgtcttgcaaacatttcaaggtctttggcaaaattttcacactgtcggaaagtcagctccgaaatggccgcaatggtctgtttgctgaacttgcatctctttgtccaatgcaactttcctcgcaaagacaacccacagtatagtgaactgctgcctttagcctcgtgttcttgcaacttttctgaaggtttgaacttctcaaaagacaaggggcagggtagacagctgctgcagtgtaactttgtagagaggaaactcccgcgtctggagctcattcccgcgtttcttcagtccgtcctctttaaggtcgccactccgggtcttgcttttaagcctcttcacggcaacgttctcagcctctgacgccccatatgcccggcgctgatgaaaggaacgaatgggatgcccgcagctaccctgacaagtgattattcttatgacgattttgcagagaagaaaattgcgtctgggcccgagtcgatacgatcacaagttgggcggggcagagattcgaacccacatctaaggacgagctctgggcctgactggtccgcaccgcgtccctgcatcagtaacgaggggtcgacgcctccagggccagcgttccagagcttcggcccgaaccgcccaccgcgtacgggcgtcacctggaatgtctaatgccgacgtttg

SEQ ID NO: 110 - DNA, 831 bp, Homo sapiens
ttttttttttccccaggagaggggagacaggaaatctgtatctgctacttttcataagtcaccagcaaatatttttaactccttttttctctcaatcttcaaatccaatcaatttccaagacctttagattccacttttgcagtttctcttcatcacatcaccttctttccaatccctctacaattatcaagatttcctttcttcttcagtaaccccacccaccaataacttcttctcatgccaggtctctcccttactcaatctatcctataattagcaccaagttattaggttacaattccctcatgtgtaacatggagatagaaattactgtacagtactgatttgaggattaaacatgattatattaaccatctaacatagttcttaacatatagtaggaactcaataaatgtcacttctctttttcttactctctgtgaaatcaaaacagtgacctggcattcaggggcctctgcaatctacccagttccctagctctgtccatcaaatattcccctagctgtatggtgcacgttccaaccaaactcatactgtgtgcagtattctttgacttggcctggcatttttatgcagacagatcactgctcatgctttccatccttacattttgagtgacctttccttccatcttccgcctaagtcttactgaccctcaaaggtttattatctcaatagctgctgcctcagaaggcctttcttaagtgcttcatacaagtgagaccttcccctctctctcaatcccctgaaaccaatggactacgctgacagagccttttggtcagtctataaatatgtgcctgtatcga

SEQ ID NO: 111 - DNA, 866 bp, Homo sapiens
  variation 859: n is a or c or g or t.
tcgatacagggcaaactgagttgatctgcatgagagagttaacaggcatttgggggctggaagctgacagaccagagaccgggaaagtctcggaaaaggttagcagcagagtgagaggtcaagaagagagcacaagtcagatcctggttaggaagctcccatagtggcaaatgaccaaggcgtttctaggacagtaatgcctcatgtccagccttaaattgctacctaggttatgctgtggtgtcgccttttgttaccatcaaacctccaataaaactgcataacacggccgggtgcggtggctcacgcctgtaatcccagcactttgggaggctgaggagggcagatcacttcaggtcaggagttcaagaccagcctggccagcatggcgaaaccctgtctctactaaaaatgcaaaattagctgggtgtggtggcacatgcctgtagtcccagctactcaggaggctgaggcgggagaattgcttaaagccaggaagcagaggtggcagtaggccgagatcgcgccactacattccagcctggacaacagagcaagactccatctcaaaacaaaagcaaaggctgggtgcagtggctcacgcctgtaatctcataactttgggtggctgaagcaggcggatcacatgaggtcaggagttcgaaaccagcttggccatcatggtgaaaccccttctctactaaaaatacaaaaaattagctgggcatggtggtgcacgcctgtagtcccagctacttgagagactgaggcaagagaattgcttgaacctgggagtcagaggttgcggtgagccgaggttgtgccattgcactccagcctgggcaacaagagcgagatnccatccg

SEQ ID NO: 112 - DNA, 495 bp, Homo sapiens
cgaaattcaggcgatttattgtaaacaggataaccctagcactccaccatactggctgtgtcacatgctccctctttcacatcaactggtcaaaaaagaagactttcttaagaagcctcaggtaactaataacttagttccacaaatctcatttactaagcactgcgtaaactgcagtatctggggtagtctgttattgctcatggactgcagcttggacccatctaaatgaatccattagtatgcctgagtgatcaacagcagctctagatccaacctggaaattggaaaatgctgacgcataccaaacaattgcaggttcatggaagactacatgagattgctctcagatctcttcatgtgtcattctcataaaaaagacacttaaatttcacccattttttaaaaaagagctaaaagttactcaatagcacataattttccacataaaaggtaggtggatttttatttactagtacagtggtggatcgcaac

SEQ ID NO: 113 - DNA, 360 bp, Homo sapiens
cgggacgtgcagtgaccccacttggcagcagacactcatttctgatatttttgtatgccaagtcttgggtaaaacaactaagtgatctcttaaggacccaggttccttttttgtccctgttccttgcccctcaccaccactttttccatgtgccaccctctcataagaactcagaagcccagggtggagtcaaaggggtcttttaaatcccctagtggcatatgaaattctggatgtttgtgaattttcctggggaaagggtctatgtgtgccattagattctggaaggggtgtgtgacctcaaaaaaaggttaagacccactggaccgagtcctctttaaatggaagtgcatggatcag

SEQ ID NO: 114 - DNA, 468 bp, Homo sapiens
ctgatccatgtgatagaaactagggcagcttatcaatttcatgtggcgtaagttagcagtgggtgggctggagctgaaggatccagtatggccttgctttgatgtctggtgcctcaacttggatgcttcagtcatctgggggctggcttgagaaggctggtcctcattcatgactgtaacacttgataggaggagttgttggtagccagctttgtagccaaactaccagagctcttaaacaaatcgtacatcagggagaaagcttaaatcttgattttaaatgactgttttgggcattactttcagaataattattagaagtgccttgattaaacttagataactaacgttaaaatatacgaaatcagatagtaaagtgtttcttgggaaatggttgaagtaaccagttttgatattatgtttgataggtgcaaaaattagtatttcgccaggggcggtggctcacgg

SEQ ID NO: 115 - DNA, 404 bp, Homo sapiens
cggcagagctctgttacagatatgaaaacaaaatgtgacacaatgaagaagctgcataaaaatactgaaacctaaggtatctgtagtatataggatgtaccctggcttaccatagaaaagtctttagatagtaaatagcactaagtctaaaagcaaggagataagatttcatgttggatactgtttcaagaactgtatttggaatattaaaggaaatccttatgttagtaagtttaaaaaaccaaacatcatatacaaaataaaattttagtatgataaaacctagcttttcttattttttaaaaggacatcaaactatataattgctcaagagcaaagtttagtgttttggtgagctgtatttcacgttacttgtagcaattatatttggtgacatggatcag

SEQ ID NO: 116 - DNA, 731 bp, Homo sapiens
ctgatccatgaccgggtaaaaaatgtcttgaaatcacatcaggctcatcaaagacatttatatgatgagagagacctagagaaagatgatgaactggaactgaaaagaagtcttttatgtagagactctgcctatgacagtgaccccgagtatagcactgttcacaggagcaaagtcttggaaccaacccaaatgcccatcaacgatagactggatacagaaatgtggaatgcaccatatgtcctcatctgaaagtctctgcaggatggtctgactcacgcgagtggttttgagataagttgtgttatacctcaagttaggtaaagtcgagagatgaactaacttaaaaagtagtgatgtttacatagaatctaaattggaaaagacaataatatcagcattagaaagtttgcctttaagtaaaaaaaattattagccataataggttttacattcttcgtaatagaatatcctaaagcaataaaacctccactatgtgaactctaagttctgaatatccgaatttttcaaatagttctgagttatatttaatttgttctttactgactagaacacttcctaatttatcacatcatttttaacctcctcaaacatactgaatatattttttccaggtttattgaggtataattgacaaacaaaaattgtgtatgtaaggtgtacaatgtgatgttttgatatgtgaatacatttgatgattctggcttccg

SEQ ID NO: 117 - DNA, 676 bp, Homo sapiens
cgtccagggttctttatttttgtctgctgccaatgtcgtctcgcatgcctgcaccctcgcatgcacgctgcccgcatgccacgtgccacgctgtagccacagaccccttgctcgggcctcacccaaggccaaactccaaacacaatcagaaccagccaaagaagcacttcctgggcacggccaccagctctcccgcctccagtgtgggccggctcctgcagggtccgagggctgcatctctaccagccagcccagggctcttcccagggtctcgcattcaagggcaattacattttaaaaagaaaaacagaaaaaggttaatcacaaaaccaaccctcacttcacagggtctgtaagtcactcatagaactttgctcttcccgagacagggtcccttccccagctcaggcacaacagagtctggcaggctctggcaccctgggcctcctccgggagcctcccatttgggcagtggagccataaacggggatccgagaagagagtatccactttttttttacaggaagaagggactcacagcataaacgggggtgggggggatcctgattttgaaaataatctatttgtagcttctcttctatcaaaaccaacacatcctcttctttctgccaatcctctcccccacgggacacctctctggttcgggaccaatcca

SEQ ID NO: 118 - DNA, 280 bp, Homo sapiens
cgaatgtaggttgttttattgtaaatgtcatcaaaatccagaacagcagaaacatattaatcagtttgaaattttagaaatcctttagcacttgaaaaagagtattacaaatgcatctatatcacatagaaagtcagcgaatacaaactagacaagcaggacatagttcttttctggcattccaggataataagaatatttatcaattaaaaggtcaatatctgtcttcctgaaataactccaaacctgagtcaacacacattcttttcggattggttcc

SEQ ID NO: 119 - DNA, 282 bp, Homo sapiens
ggctagtcaagtgaagcaacaggaatgtagaagaacaaataaatctgtaagtagctgtgatcaattagttgtaaacagcaccgcacttggaccagcctgtggtcacccttaacatctatccaaaagctgatgaaagcagcagcctgaaggcatcacgtggtacaagaggagcaggtaggcctgaaagagcaccccaccccaacttcccctttctttcttattctctactatttctcacatttctttctactgcctcactggttcttacacaggattggttcc

SEQ ID NO: 120 - DNA, 317 bp, Homo sapiens
ggaaccaatcccagtgtccagcaagaggggtaaagttaagtaaattatggaagattcacatggtaaaacagcatgaagccattaaaagtactatttctgaagagttcataacatgaaaacatggttatgtgaaaaatggggcacaacagtttatatactgtatgaagataaatataaaatatgcggccgggtgcggtggctcacgcctgtaatcccagcactttgggaggccgaggcgggcggatcacgaggtcaggaggtcgagaccatcctggctaacacggtgaaaccccgtctctactaaaaatacaaaaaaa

SEQ ID NO: 121 - DNA, 453 bp, Homo sapiens
cgagacagggtctcactctgttgcccaggctagagtgcagtggcactatcatggctcactgcaaccttgacttctcaggctaaggtgaccctcctacctcaacctcttgagtagctgggactataggtgcacaccactatgcccagctaatttttgtatcttttgtagagacagagtttcaccacgttgctcaggctggtctcgaactcctgggctcaagtgatccacacacttggtctcccaaagtgctagaatttcaggtgtgagccaccacacctggccaatacctacttttctttcatgtctccaaggaggtatcatttccttatggaaatctttcctaaccactcaagactcggttaagtgcctctcctatgtgcccctgcagcaccattaatgaatttcccctatcacagtatggttatccctcagtatttgttggggattggttcc

SEQ ID NO: 122 - DNA, 491 bp, Homo sapiens
ggaacccaatccaaatgtccgtcaatgatagactggattaagaaaatgtggcacatatacaccatggaatactatgcagccataaaaaaggatgagttcatgtcctttgtagggacatggatgaagctggaaagcatcattctaagcaaactctcgaggacagaaaaccaaacactgcttgttctcactcataggtgggagttgaacaatgagaacacttggacacagggtggggaacatcacacagtggggcctgtcgtcgggtcgggggctaggtgagggatagcattaggagaaatacctaatgtaaatgacgagttaatgggtgcaacaaaccaacatggcacatgtatacctatgtaacaaacctgcacgttgtgcacatgtaccctagaacttaaggtataaaaaaaaacaacaaaacttgggtctcagtacttagaaaatagttaaccatgatcaataaaatatttgccaattccttcaactcg

SEQ ID NO: 123 - DNA, 255 bp, Homo sapiens
atggcccacaaatattccttttgaaaatgaacccaaatataaacttaatatgcacagagcaggcacagacatccgcgaaatgtcactctcgaggatgaacatatacatactatacacaaagcaatcaatcatgacgacagagattcaaccaaatctaagttaactgtcgaagaaaggtaaagatggttttattagcttcattaaactagtaactgcaactagagttatcctcagtattctaaggggaatgggttc

SEQ ID NO: 124 - DNA, 403 bp, Homo sapiens
aatgttcattactttaaaagcttacatttaggaaactcaagctttgcttttctataatggttaacttttccatattatcataagaatcaactttctgattaatatttctcccttttttcttaagaagtcaatagttcttcatatccacagtcatgagtcatttttgaggacatgccagaattaccaatgtaactgtgaggcaggaaaagtacactcccagggaagtcagagtaagcctgtttccaccgcagcacagcagtgagcacagctaggcagaattccagcagagtgcaaatcagcatcagagagagagttcccagggcagacagagcactcagaatgcttccaaccaggctgctatgcaccaaaagcttggttaaccttttctctgtggcgattgatc

SEQ ID NO: 125 - DNA, 523 bp, Homo sapiens
cgtagagatggggtttcacttgttggccaggctggtctcaaactcctgagctcaagcgatccgcccgcctcggcctcccacagtactgggattacaggtgtgagccactgtgcccagcctaaaggttttaattacagaaaatctcaatcttctgcatttaaaagatcagtttgactgttctgtaaaaagtaagaaaaggacagagaaatactttaggaagctattgtagcagtccagaatgagataataatttgaactacagttcttgatatactttgggtatctgtccccacccaagtctcatgttgaattagaatctctagtgctgaaggtggggcctggtgggaggtgcctgcatctggattatgggggcagatccctcatggcttggtgcagtcttcacgatagtgagttcttgtgagatctggtcatttgatagtatgtggcacattccccgccccgccttactcctgctttcaccatgtgatgtgcttgctccccctttgccttccactatgaccga

SEQ ID NO: 126 - DNA, 465 bp, Homo sapiens
cgaggaaagatggggtttcaccatgttggccaggatggtctcgatctcttgaccttgtgatccacccacctcggccccccaaagtgctgggattacaggcgtgagccaccgcgcctggtgtaagtctgtatttgtattctaaatgaaatcttaggacactgaattaaatttagtaaaaggctttgaggagggtaccaatggggatccagaactcgctaatgctctttacaaagctgacatcaggaaagtaggcatacacactaggtgttaactatgtaataatgtaaaattatttgaaatggattaatgacactaaatttgaagcaaaccaatatagtttaatgggtttttttatattttcataaatattttaaaggttttattgatcatgtttcatgttgcttctttgacttgtgagtagcatggagagtaaagtttgtggagtaatcaagtggctatgaccga

SEQ ID NO: 127 - DNA, 417 bp, Homo sapiens
cgttcaaagtagctaacattgcctttggtcttttaataaatgattctgaattgttgaggaattctggtttttttcttcttgttgttgttttgttttttttagatagggtctcaccctgtcacccaggctggagtacaatggcacgatcccagcttactgcaacctccgcctcccaggttcaagcgatcctcccacctcagtctctcgattgcaggtgtgagccactgtgccttgctggaatgctactcttaatataaaacaaatactatgtttactaaccacattcattcttgggttcactgtatcctttttgtcccactagagtctaccttctgattctccccttccaagcaaagagaatcttgatcctttactgtcagtcttgattcgagtgaatgagtactggcctatgaccga

SEQ ID NO: 128 - DNA, 403 bp, Homo sapiens
tcggtcataggctctctgcagggggaagcacatcacacgctgttggctcattctggcagtccaacctggcattgtctttacacaatcctgcatgcaactttgtattcacaataatcaggagcatttcatcttttattccgtagcaatagtttcagggggtctgcctacaatgattattgtaaaaattcaaataaaacaaaatttttatcttagaaactgaaagttcccctgtcatctgaaccttaaagacaactattgttaatatattattatttcaaaaattgtgggccagcagtggctcacgcctgtgatcccagcactttgggaagctgaggtgggcggatcacttgaggtcgggagttcgagaccagcctggccaacatggtgaaaccccgtctctacg

SEQ ID NO: 129 - DNA, 400 bp, Homo sapiens
cgtcaatagcatttctttccatttatggtacaaacattgtcattaaatacatttataactctttccataaaggtcctatatttcctggttcacattactgtaaggtctcatattttcattgtttttgtgaaaggtatctctattttttaagttgtatcttttaatttttttatgatggaaaatttcaaacatacacaaaagtagagagaacagtacaatgacccctaggtaccatcagcagattcacagcttatcaatttagagccagtctcttctatttagggttgccagataaaatacatgttcttgtttctgggccccctactcctttctctaagtttatctgtctacctttgttgtgctagttcaactctgtctcaatcagtacccctatgaccga

SEQ ID NO: 130 - DNA, 390 bp, Homo sapiens
tcggtcatagcaaagaaagatgagggatagtaggacagaggtatattttgtacatagacaatcaaaacaaagataaaagtcatgagtatgattacaggcgtggtggctcacgcctgtaatcccagcactttgggaggccgaggcaggtgaatcacctgaggtcaggagttcgagaccagcctgactaacatggtgaaaccccgtttctactaaaaatacaaaaaattagccagatgtggtggcgcacgcctgtcattccagctacttgggaggctgaggcaggagaattgcttgaatccgggaagcggaggttgcagtaagccaagatcgcaccattgcactccagcttgggcaacaagagcgaaactccgtctctaaaaaagaaaaccg

SEQ ID NO: 131 - DNA, 307 bp, Homo sapiens
tcggtcatagtcacatatggaattttgagaaaataaagcatgctgtctttaggaatttttatacttctttgtctttcttccttaatatttgcttctagctgctcttggcaatgatgaattgttatgtatgcattaatgttttgcagcccaaaagttgttcacatttttcctatataagatctgtggagtgtgtgtttcaaagagagaactacagaaatgttaaagcaggaaaacctgaatgtgatgtgcacattttcatcccacatggacaatgtatgtgttttaataaatggaattttcagattcg

SEQ ID NO: 132 - DNA, 678 bp, Homo sapiens
  variation 651, 674: n is a or c or g or t.
tcggtcatagagtgaagaagttaatgggtaaataatctgacactgataaccagtatgaaaataatagtgttaacagtactttaaaaatggcagtggaagcaacccaagtgtccatggatggatgaatggattaacaaaatgtggtatgtacatatagtagaatattatttagccttaaggaaggagtctgacataatgctacagtatgaatgaccttgaagatactattctaagtgaaataagccagtcacaaaagtataaatactatatgatttcatttatatgaggtacctatctatagaagtcaaattcagagagaagtaaagtagaatggtagttgctaggggttgaaggagaaggggtaggaaatttgtatttgaagagaacaaagtttcatttgaggaagatgaaaaaattctggggatggatggtggtagttgcacaacagtgtgaaaatatttaataccctagaactgtcacttaaaatgggtaaaatggtaaattttatgctatgtatattcatggccagtaaaagtaatagcaaaaactgcagttacttttacaccaaccctaataagcacaataaaaagaatttaaaagggcctggtgtggttgctcacacctatagtcccagcactttgggaggcttangtgggaggattgcttgagcccangaat

SEQ ID NO: 133 - DNA, 873 bp, Homo sapiens
cgtagagacagggtcccactatgtcacccaagctggtctccaactcctgggctcaagcaattatctcgtctcagcctcccaaagtgctgagattacagatgtgagtcaccatgcccagccctataacaaaagtttagagatattagtgatcttctaaatcctatgataaacgcccttgaacaaatcaataaaataccaaatatgaactgttggcagttttaaattgcattttcaaaatgcagaaactgttacgttttaagaaggaaatgttctgcaacctccttttatcacttgactttttatatttggcagacagtcccagtaggcaaagtctggcttccagaacacagactgttggcatttggatctcttttatcaaaggagggcaatggatgtccattacaatttagattggaatttcctacaggattctttgcaactccttcctgcttcccagctagctttgagggagagcttagctagaaagtgaaatttttggatggaattcttatctacaagcaagataaaagagtaattaggtagtcctttcatattttagtggggacatagaaaatgacaagtcatcttgtaaatcaaaaacaaaaataaatatgcacaggataatggaacttgaatctacctgacaatatgaaggcaccttgtttgtataaattttgagataacttaatccagtaccatgtgctgataaatcttggataaagagactcttgggaggcaataggctccgtatttcttaatgctacatgacatctattagaagcttttggtcttgggaaattgtggccaattttattttctacagatggccacacagtatctccaatcccatagattcttatgcactatgaccga

SEQ ID NO: 134 - DNA, 620 bp, Homo sapiens
tcgggtcataggcatgtttcagtacttgatagcaggagattacctggaagaagtcagagagccatcttgaatgactgtggagttagctggttttattatactcttgggtgacatttccagctaataaaattagaattaaaattataatcttagaagcaagcctctgggaaatatgtaattaaccttttggaatttaacaagcattgtgtgtacctccagtacctatatttaagatgctttaacctagcatttgaattgatgccaagatgtgcaagacatagtctctgccctcaaaggctattaagagacagactatatgtacacagatttttttaaattatttaggtttaattcataaaaaccctagaagaaaacctaggcaataccattcaggccattggcatgggcaaggacttcatgactaaaacgccaaaagcaatggcaacaaaagccaaaatagacaaatgggatctaattaaactaaagaggttctgcacggcagaagaaactaccatcagagtgaacaggcaacctacggaatgggaggaaatttttgcaatctgctcatctgacaaaggactaatatccagaatccacaaagaacttaaagaaatttaccg

SEQ ID NO: 135 - DNA, 545 bp, Homo sapiens
aggagtgctagggctttattacaaatggagttgactgctagagaggcccttctccaatctttcttctgtaccttcttccctcccaaagacatccctctaggggaggtcagtaggccattaggtaggaggaaatctggagagtgaaaaggggccttgcttttgtcaaagtcctctgaaacaaccactgagtctgaaggctggctccagttgagaatcttctagtggaagaggtttagctctcatcttcaaggtccttcatttctacatcctggggggcttttgtcttcttttgccttttgagctgtggttcactagtcctggctggctttgaaggggcttccacttccatggctgtcttctctttctgggcaagccggatctgctggaggagttttctgcgcttcttccctgacagtgtaatgttggcacgtgcactggacgcccgcttcttgaggtggtgccgcgtgatcagcccttggtctatcacagccccgaccacccggtgcctcagacgccgctcccgactcaacacccgccgacgtttg

SEQ ID NO: 136 - DNA, 575 bp, Homo sapiens
cggtctaaatgatcagtttattgaattttagtaatgtaagttgagatcatgaaacatacaatctaattcaaaagaagaatcataggtaccaattcaacaccattagcactactttcacaatagtgacaccccacttataacactcttttttgggtctctgactgtgaacaagattgtggaaggttggttgaggttaggcctaggcattacggttaagaaataacagatgctaattagttagtggggaagtgaagctcatgtttaggccttttcataatgtctttatgctaaataaattaaatgaagtaaacagccatggctattgaactacagaatacataaaaatacattctgtgattcacctataaacaaaagagtaaagcagaatgtgtcaaattttccatagcaggtaaagcatagaaaaaaaatctagctgatatagttgttatgtttcaacaacataaaatgaaatgtttctaggatagaagaaattggtaccattaacatttggatggttatggtaccataacattttgatataatatgcaaaatgccgcctccccaactatgaccga

SEQ ID NO: 137 - DNA, 546 bp, Homo sapiens
tcggtcatagcactggaaagaaagaagcagccttaattctcctaacattgccgtttagattagttggagacaaattcattttccattaagtcttttattcattccacaaatatttcatgagcatcaaatgttatgtccaaagcaccatattaagtgtggagtgtaggcaaagtaaataaataaataataccgaaatgataccaaaaacaaaccccccaccaccaacaaaaaccaaagcagaacaaacccatttataatagcatcaaaaagaatacttaggaataaacttaaccaaggaggtggaaaacttgtacaaaaagtataaaacattgctaaaagaaatcaaaggtaccaataaatggaaagataccctatgtacatagattggaaaatttaatactgttaaaatgtcaatactacccaaaacaatctataaatctatgtaatcttgatcaaaatctcagtggcaattttgcagaaatagaaaaagctattctaaagttcatgtggaatctcaaggaaccctgaatagtccaaacaatctcg

SEQ ID NO: 138 - DNA, 516 bp, Homo sapiens
cgcagagatggggtttcaccatgttggccaggctggtctcgaactcctgacctctagctaggttttaattactaactggcataactaccatgagcctctcccacgtttctagcacttaaaaactccagatttttcccttagaaaactactcagaaaaaaagtttagctaattagttttcccttgcttgtgaagtaacaaatgcagtctttatattttttgtagttctggtggtcttcgttctattccctgccaatgctaggacctgggggtggagcaggggtaagggagaagcacccccgacagctggaagtacctggctgccaccggcgaggctgtggtgtcccctggtgaacacaaacaagttcacagggttaagatcagatgaggactcaccatgactgcgctggaacaggatgaaagccaatcagcggccatgcctgaacagaggtcttcttttttaaaagggctctatatgaccacaaaaatgaacaaatgtccctctcccctatgaccga

SEQ ID NO: 139 - DNA, 452 bp, Homo sapiens
cgacttgaacaattggtttatttctaaaaagggtgacatttataagttgggctagataagtaacaacatagaaaaattatttcctccgttaacaagagctaaaacatgtcaaacatatcacatacaattcttctattatatagctatccagttggaagttttttttaacacttatgtacagggcactgatctaaccatttatgtgaatcaatttacttaatcattaataataacatgaggaatatgtttcaataatgcagaataccagaaaattatagttttagcataaaaaaatttttttttttgagacggagtctcgctctgtctcccaggctgaagtgcagtggcgtgatctcggctcactgcaagctccgcctcccgggttttatgccattctcctgcctcagcttcccaagtagctgggactacagacatgcaccacctatgaccga

SEQ ID NO: 140 - DNA, 419 bp, Homo sapiens
tcggtcatagcaccccaaaatgtttttggttatatgggtttatatctatagatactagtttagaaattaaagctgagaatttaaaaaatacattggagtttttgggattgctttttaatgaattaaataagccaataagtattaacataaataacatcttctgaaaaatgactttttcaaaacaaaataatgagtggcattgttttacatttttgcacatcacttcgtgtctgttaataggagatagtgggttttcatgtctcctttgaattcagtgtgttgcggagtttattcagagatcctttagctgggcatggtggagtgtgtctgtagtcccaaatactcaggaagctaatgctggatcgcttgagcccaggagttcaagagcagcctgggtgacataatgagaccccatctcg

SEQ ID NO: 141 - DNA, 390 bp, Homo sapiens
cgtagagatgatgttggccaggctggtctcaaactcctgggctcaagccatctgaccgcctcagcctcccaaagtgctgggattacaggtgtaagccaccacgcccagcacaagtggacttatttttaacagattcccaatgcctccaggataaaatcccaaattctaagcctgtgactccagatatcacacgagctgatccttctgcctccttcccttacaattgaatttattatttttgcactaagagtccttcatgaatagcttccactttcccttcatgtctgctcaagctgattcaccttccctttcctttcctttttctctacatttatctaacttcctttcttccttctgggcaccgttcaaactggatctgcctatgaccga

SEQ ID NO: 142 - DNA, 321 bp, Homo sapiens
cgcttattgttttgtttgttttctgtagagatggggtctcaccacattgcccaggctggaggcttcatttctaatctttctatcccctcttcattggctttgagatacccctgctgcttcgggctcctgagggccttcagggccccacattaatgaggcagccacctagcaagtctgacatggctgtcagtgacccactgttccagtctctctccatgttgcattccacccaacgggaagcagaagacaccgtccggcagttggaggtgctctccacataaagcgacttcccctggtgcttcccagccaccctatgaccga

SEQ ID NO: 143 - DNA, 319 bp, Homo sapiens
cgtagaggcagggttttgccatgttggccaggttggtctcgaactcctgacctcaggtgatccacccacctcagcctcccaaaatgttgggattataagcgtgagccaccacgcccggccttatttggtaattattttccttacggatgcattacttactgccttttatactttgtttcagcttttttaaaaagttaaagtaattactgtatctcaatttcaaaaggggggcaggggtggttgtgaaaggaaggtatgtatggctttgacatttggttttataaacttacatattgtttggattctttactatgaccga

SEQ ID NO: 144 - DNA, 772 bp, Homo sapiens
cgcaagagtgaaagacacctatttaatggcaaaatagtgatggaaagcagcagaagtaaaaccctcatttttcctataacagacaaatcgcttgaaaatccagtttaataaaatcccatgatccctctgaacaccagcttcctgatcctcatctctaccgatggcttcattcatattccttttccatctcatttcctgtttttttcgtttctcctatttctccaaattgtgtttttcccagctcttctggctctcttctgcccacttctgaaagtttctttagttcttctggttcctgtctgctgaattctcctgagagtctttgctctaattctcccgaaggcctcctataaatttctcttgactctcttctgccagcttctagtagcttcctgcctgtttctcctgatcttgtgcctaattctcctgacaggcccttggcaactgctcccaaatcgcttctggcttttcctgcttccacactctgctttccgcttgctgggctctgcaaagcttccccaaaagcctcatcgtcctcatctgtctctttctcatcctcactatggtcccactgcttcaccaccttctcgggctcatctctcactctcctcatgtccttctctcttgttattattctatcttcatacagtgaagcttctccctcgcttttttctttgagattctccacgtagttggttttcctcagttctctgatgatacccttgttggcatcgtccaaggcactgatgatggcctgctgcgcctcgttgta

SEQ ID NO: 145 - DNA, 474 bp, Homo sapiens
acaccacgcccagccatctcctctagaattcttttcatgtcttttgaatctcctctctctctcttccatattcttgcctttttttttgtttttatctctttatcatctcctctgagttttgaaacaattcaaacttgtctgttaattcaccaatttggcattttgcagagtttaaattggctgttactgcattcactgtgttttaaaatctggaactatgttttgcacttctaagctatctttcctgaacacaaatggttcccttttagaactgctgacaaattaccatgatacaacatcttaccaaaactttaagcaaaaattacaaattaaaaatgttctaagttttgcttgcctgtttcttgcagcagctttaacaaggagactttattctgatttttcagattggtcccttgtctttataacagctggctgttcataaatgtttggtgaatcttctgtttcctcgttgta

SEQ ID NO: 146 - DNA, 277 bp, Homo sapiens
gtagagatggggttttgccatattggctgggctgggttggccaggctggtctcgaactcctgacctcaagtgatccgcccaccttggccgcccaaagtgctgggattataggcgtgagctactgcgcgcagcctggttcatttatacttgacataaatccaaaagaaggccacccctgcacgatctccaagagcctctgtggaaagctctagtttaaccctaggaaggagaacagctcacctctggcatgatctcgatcttctcgtaccgacgtttg

SEQ ID NO: 147 - DNA, 305 bp, Homo sapiens
ggtgatgcagatttcaacagtaactctggaaaactgtgaaaaatgttatttaaaaatatatatgtatatgctactgcacagtttcaaagatgtgattcataaataatgttggctgcactgattaattttataacaattactgcacttccaagttgatgcgaacacgcagtgactcatactcaatattaggcactagtaatatccttcaggcgtactacagttttatgttagctgtattgtacatatatatttttaaatgtatgcatttatacaaactgtgtatattatgtatggggtgtcagatc

SEQ ID NO: 148 - DNA, 457 bp, Homo sapiens
gatctgacactggaaggcaacccactcgcatgtggatgctgaagggcactttattttgtatcctgaaaagggcctacagaggccagtacaaaactctcaaacaaaaggaagttgactctgctttgaggggaaggtggcatgccagcactagcttaaaaagggaagtgctagccgggcgtggtggctcacgcctgcaatcccagcactttgggaggccgaggcgggtggatcacgaggtcaggagtttgagaccagcctggccaacatggtgaaaccccgtatctactaaaaatacgaaaaaaaaattagccgggtgtggtggcgggagcctgtaatcccagctattcaggaggccaaggcaggagaatcgcttgaacctgggaaggcagaggttgcagtgagccaagatcgcaccactgcactccagcctgggcaacaagagcgagactccatctcg

SEQ ID NO: 149 - DNA, 222 bp, Homo sapiens
cgcctcttttacccatcagtttccacagctaccatgctgtcttttagaatcatactaatgtcgccaagtctggcaaaaccatggatatattctcacacgcagatgtgatgtcaaatctgtctatcctctgcccccaaactctccacataatgaacacttttcctctgcaaacatgtattttggtttgtggaggaactcaagtcattaatgaggtgtcagatc

SEQ ID NO: 150 - DNA, 217 bp, Homo sapiens
gatctgacactaattctatggctggagcacatcccttaacctctccgagactccattttctttattataaaattagaatttggaccagttggcctctaaatgtcttccatctttgactctttgtcttttctgtgcacataaacatcagatctaatccttgttagtggcattattttgaaaacatgggacagaaacataggaacaatattctttatct

SEQ ID NO: 151 - DNA, 740 bp, Homo sapiens
cgtaaacaatagaaatttattggccgagcatctcagcctcccaaagtgctaggattacaggcgtgagccaccgtacccggctggatttacagactttatctttattttttcaattataaaattattcacccacttagaacagaaagcaatatggaaaaaggaggaaagaaaggtcttctgtacttggatggggccaggttggggcaaggaactggcactgtggagcttagtgcttgcttacaggaccagcaggctatgaggcaactgacatgccccagggaaagtcatcttgagttgcttcttgatagtgagtgggatacgggtccaagctgcaaacttcttaagtcattctctaaaacatccacacttcaggactgctattgggtgctatgttccagtagattgggtgactcagacctcagcatcacacaatatatccatgtaacgaccctgcgcacgtacccctggaatctttatcctgtcaagccgtcgctttatggctcgactgtttttatctagctcctctaacatcttataggggttgaaggaaagcggtttctcgtggtccacaaagttttcccagcagtcagcaaactctgagagggaagagggaaaccagccaggccccaggggcagcagctgcagtgccaggagctcttgcttcctttcccctctcttctcttcctctccctggacattgctctgtccggggaattctccctcccgggtggtgtcagatc

SEQ ID NO: 152 - DNA, 327 bp, Homo sapiens
agggtaagataggtatttggaccacatctgagatagttgacctgggctctatggaaccccccatcctgccaccagtctcatcgtccatggatggatttcaaggagttggcaaactaaacctgtaccacctgtggaacacatgcacttttctaagcaggaggttcagagacaatatcagaattttaaagaagtctgtgataaaaaattattgacaacaaacaaatgaataaaattagaatatactgcctaataaatacctaggatcaagtatttttaggattaaatgtggttatgtaacaagaaaagctccttgcccagtgtcagatc

SEQ ID NO: 153 - DNA, 503 bp, Homo sapiens
gatctgacactggagctaacactcataaaaagtgagacagtactttcagtctgaacctaagtggattaatttcctggtaaaacaaacaaacaaacaaacatttttcagatgatttttaacaggattcagagtctcacaacatattaaaaaatccaggatacaattcaaatttactcaatatcaaaacaacctggaaaatctaaacagtactcaaaggaaaagacaattcaataggcagtaacactgaaatgacttagatcgtgaaattattagacaaataaagtaggtatgaccattctccactcttgaaattagaaagttcttgaaattaggaaagatagaagttctcagcagagaactagaaactgcagaataatcagatagaaattttagacctgaaaagatgatatgggcccagtagcggaataaatataacagagaaaagagtcagtgaacctgaaggtagatcaatagaaagtatacacaatgaaatatacattacg

SEQ ID NO: 154 - DNA, 714 bp, Homo sapiens
gatctgacaccaaaatcactgcctattaaagaaaaaaatcatagtttggactttaccaaaattgaaaatatttgctcttcaaaagtcaccattaagaaaaataaaaatacaggcacacatgagagatactgcaggtttggttttagaaagccacaataaagtgaacatgtcagtaaagcaagtcacatgaatttctggtttcccagtgcttataaaagttacgtttacatgacatgtgtgcaacagtgttatgtctaaaaactatgtgtctgaattaaaagtgctttactgctaaaagctgcttacacagacgcttgaagtgagcacacactgcggggaaaatggcgccgacatacttgcttgaccagggtcaccacaaaccctcaatttgtgaaaaaacacagtatctgtgaagtgcagtaaagcaaagcacaataaagccaggtgttcattcctgtgcaagccacaggcaaacaaaatattttcaagtcttagactgatgtccggaatacacaaagacacaaagagctttcaaagtgcaattgtaaaaaaacaacaaacaaacaaaaacataaacaaacagaaaacccagtttttcaaatgggcaagagggctggacacaatgggaagcacctataatctcagtgctttgggaggccaagcaagaggattgcttgaggccaggagttcaagaccagcctgagcatctctacg

SEQ ID NO: 155 - DNA, 499 bp, Homo sapiens
cggagaaggagtttcgctcttattgcacaggctggactgcaatggcgtgttatcagctcaccgcaacctctgccactcgggtccaagagattctcctgcctcagcctcccaagtagctgggattataggcatgtgccaccatgcccagctaattttttttttttttttgtatttttagtagagggagtttcttcatgttcatcaggctggtctcaaactcccgacctcaggtgatccactcgcctcagcctaccaaagtgtggggattacaggagtgagccaccgtgcctggtctgacaaattcattttctgtttggtttggtctgctctgtttccaacaagagcctacttcattatctccccaatatatctacctagtccatagactggctcagagcaggcctttgacaatatttaattttatcccacaatggcatttcaattcctttgtcattctgagaaaaatactaacagcatgggaatttggtgcatggatcag

SEQ ID NO: 156 - DNA, 499 bp, Homo sapiens
cgtgagcaataaagctttttaatcacctgggtgcaggcagactgagtccaaaaaaggagtcagcaaagggagatggggtggggcagttttataggatttgggttggtagtgggaaattacggccaaagtgggttattctctagcgggcaggggcgggggtcacaaggtgctctggtggggagctcctgagtctaattgtccaggagaaggaatgtcacaaggctaattgatcagttaaggtggggcaggaacaaatcacagtggtggaatgtcatcagttaaggcaggaattggttattttcacttcttttgtggttcttcagttgcttcaggccatctggatgtatacgtgcaggcttgggctcagagacctgacagctccctaatttgcttctaaatattctaagggagaaggaagagctgtgagaattgtctttgagaggaatcattctctctggagtaaatgagacaaatcttctgtgttgtggtcatggatcaa

SEQ ID NO: 157 - DNA, 485 bp, Homo sapiens
cggagtgctttcttcttattgagttttgagagatcttttactattctggattcaggccctttatcagatttgtgatttgaaaataacatttcccagactgtcacttggcctttcattctcctaagagtatctcttgaagaacagaagttcctaattttgatgaagtctaaattataatttttaaaaatgtggattgctctttgcctaaaccaaggtcacagtttctcctatgttttcttctagggcttctatagttttaggttttatattcagatatatgatccactttgagttaatttttacatatggtgtgagatatggttcaaagtttatattctgaatatatgaatattggattgttacagaaccatttgttgcaaaggctgtccttttctcactgcattatctttgcatgtttgtggaaaaccatggaactgtctttgcatgtgagtagaaaattaattggctgcctatgcatggatcag

SEQ ID NO: 158 - DNA, 600 bp, Homo sapiens
cgtagagacggggtttcaccgtgttagccaagatggtctcgatctcctgacctcgtgatccgcccgtcgtggcctcccaaagtgctgggattacaggcgtgagccaccgcgcccggccggcttacattcttttaaagtcagaagaaaaaaatgaacttttaggtcagagaaaatattttgttatataatattgatacgtcatgtttatatcaatgcagttgtaaaatatgttaaatatttatacatatatttaacttacatgtgtgtgaaatatatgtgtgtgtgtaatgtacaagccacaaggcaaaagtgctcagggtccaagaaagtcaaaacgtgcccctgcacccgaaaagcacgaaaccttctggggcagtgattatggcagagcaagttggaagatggcaggtggtagacaaattgtgaaaaatatcacatttctctcaccctgtcaaaggcaaccccaaagtgaagtcaaaatagagatgaaatgcagggtcacttcctagctgcgtgatcttggacaagagagttgaccaacctgagcctcagtttctccatggatacaatgggggatgcgttgcctttccgaacgtttgc

SEQ ID NO: 159 - DNA, 306 bp, Homo sapiens
ggtcagctggagaaagttatgtttttctaagaatttgtccacatcatataaatttcaaacgtattgccctatagttattcattagtttttcacttaacatttgaatgtctacaagaaaagtgatatgtttccggtttcctgccaaagatttcaaggttggcttttacttctctgagtatagtaagcatggttgctttatagtatacgcccggtaattccaatatttgaagaatctgcgaagtccatttcttttatttctagtcacttacattcatggaatctcatatcttcatgtgcatggatcag

SEQ ID NO: 160 - DNA, 295 bp, Homo sapiens
cgtagagacagggtttcaccatgtcggccaggctggtcccgaactcctgacctcaggtaatcccccacctcggcctcccaaagtgctgggattacaggcgtgagccatcgcgcccagtcctatttcaattctttttagtgtgtcaataagtgaaatgagttttttttaatatgtagaggttatttcagactatataatattttcttttgtctcttttaccctactggggccagaggaagttcattatctagaattacagttaagaaagaagtccagcgataactgcatggatcag

SEQ ID NO: 161 - DNA, 295 bp, Homo sapiens
cgatttcaaggcaaggcacgtaatgtggacattatatcttcgtgcaaattaggattactggaaagagtatttttaattaaaattttaagagacatagaggcaaaatgtgtctgcccatgcacactatggatctgtcaatacaagaaatttgttgaacaaggctaatgtctgaaagcaccatgcaagttttcagcaccctgattacatttgttttctcaagagtgcgtttttatatcctacaccctggcgttcccagtttgtaaactgtaagctttacccttgtgacatggatcag

SEQ ID NO: 162 - DNA, 254 bp, Homo sapiens
acaatggcttttgtacattttctcctctcgataaacctcagaatggtatccatcataccttctggcagatgaccgtgtctcctttttagagtaaatggaagttgccagacagaaacttccttgatttccttccataaaaatgagaaatttacccacatggatgccctttttggggggtctctcctgctattgcattgaaagaagtggccctcttctttatccaaggtcaatccctccacctgtgcatggatcag

SEQ ID NO: 163 - DNA, 553 bp, Homo sapiens
ctttcgtatgttacaaatattttcctactctgccctttaattttgtggtgcttaataaagtggaccacatataaacaaagtttcagtacattaaaattttcctttgtgatttttacctcttgatgtatgatcactttttttataattttatagatttcttttgagtcatgctcatatgtaagtagcttcaagctaatttagaaaattaaactatatacagttaatcatctgccaggaacaaatgtcacagaattctggcttctgttttccctaactaaaatttactggcagaatgtcaacaaaactaaaatgagagaagaaacacatgccccttagcaagtgacatccagtattgctattcaaatgaactggggtaattccactagcctgaatgaatttgaagtcagtagtttataccttttaactttaattcctcacattgccacacttctgaatatcttgggcaaaggactttcctgttcatcattgcaatgttcaatttgacactgtgagataggaaaggcaggtatcatctccctttgcatggatcaac

SEQ ID NO: 164 - DNA, 473 bp, Homo sapiens
  variation 5: n is a or c or g or t.
ggtanagatggggtttcaccatcttagccaggatggtctcgatctcctgacctcgtgatccgcctgcctctgcctcccaaagtgctgggattacaggtgtgagccaccacgcccagctaaattattcttttataaagataaaggatgctggattccatgatttgcaaaatcacttctagttctaaaactgtaactcaatgaatatctgcaatctgaaggatacttgtattagtgtaagtacctatttcagcagcaaaagagcaggacattcaaagattttaatgcccaaagccactgaaatatacttggaccagatctatttacaggacaggggctgggtcacacaactagcatctttaagccacagactcttagatgtaaaatggtcctttacttttatgggttacctaagtgaacatccctgtcccaattttatgaagtcatccttccctagctccatatggaccaatcca

SEQ ID NO: 165 - DNA, 386 bp, Homo sapiens
cgtagagatggggcttctccatgttggccaggctggtcttgaactcctgaccttaggtgatccacctgcctcagcctcccaaactgctgggattacaggcattgagccacagcgcctggcctacagttgactttttcaatttttggatgggactcagattatttctgcaagagtagtgcagttttgctactgctttattaagaactacatctctgtaaacacaagcatatttaaaaatcataatattgttttccttaagaaagaaaaacttttaataagactgatcagcaagaatcacaacatcaaataggactgattaatattctctaccagtagttcctaaacctgggtagctttttaaacatatacactactggaccaatcca

SEQ ID NO: 166 - DNA, 610 bp, Homo sapiens
cgaattttaaatttttattaaaaatcacagtttttaaaaatgtccagtgacacaatgtagctcttctgaaatgaatggaaaacacaacacactgctaaccagtggagggagctctctgcagggctgggagacctgacaatgcagctgtagcctctgcaagtgaaaatccaggccgacttgcagtcattggactgatgtccaagtgcaatcaccatacagcagctacaggcagggctggctgatagggagtatgggagaaggacacgctcagatgaaaacatgcatgcaacgattttcaccactgaacacactgttttctgtgatagaaactgtcggccctgctgggggacaagatattcacggcctcactagccagtgtgatgccaccagggcagcctgcccctgatgctcctttgttacctgctaaagaaggaccataaggtaaaaggcaccttaccttatggagtgagcccagaccccagggaaaagcttgggtagaacaatccaaggggcagcctggatgtgagaatccagcccaagctagctgctctagaagcctggaggcctttcccagcttcccaaatgtagcccttttcacatgaccaatcca

SEQ ID NO: 167 - DNA, 479 bp, Homo sapiens
caaacgtcggggctacatgagtatctgtgttgaatgcggtctgaaatgatcctatggattttcccggctggttgccactactgtacaacattcagtgcccacatccatctgtgccattaagcttttttgagacatgagagatgcctcttccctgctgtatgacatgcatttgagaagttggaaagaaatgacaaaatcagggagaaaacatccaagcttcttacctgtagatagaatcagccctcacttggtgcttattaccagttattcaagaacaataacaacaacaaaattagtagacatccaagaagcacatattaggaccaaagatagcatcaactgtatttgaaggaactgtagtttgcgcattttatgacatttttataaagtactgtaattctttcattgaggggctatgtgatggagacagactaactcattttgttatttgcattaaaattattttgggtctctgttcg

SEQ ID NO: 168 - DNA, 279 bp, Homo sapiens
tggattggtccagtgggaagtggagagcaacaagagagaatggaactcccagccgcaatgaagatttgcagagtgaaagctgccttcaacgtatcatgaagccggcgcaccatgaggaacactttcaaaagtcagtaataaatatctcaaatgcacatattttattgcacctttaaaatgcatacaaaagtgcatttgattcgttttaaatacaattatcttaactcttttttaacgccaggattaagccaattctactccataaggcttttgataccg

SEQ ID NO: 169 - DNA, 254 bp, Homo sapiens
cgctcatcagccattattagtgttagtgtattttatgtgtggcccaagacaattcttcttccactgtggcccagagaagccaaaaaagttggacactgtagcatgagtgttttgcacacttagaaacatgcatcacctgcacccttttgtccagacaatttctggacaattcgtacaaatgatgtgacttacaacgaacccccgattttcttccggggttctgtagcctcctgtgtgcctatgggaccaatcca

SEQ ID NO: 170 - DNA, 465 bp, Homo sapiens
cgtttgcatgtattccttctcttttagataattcttcttcataaaagatacttttcttttccaaatcagtctttagtttggttatctcttgctgatgttctatgctgcatactcctggtgagtaactaatttgtttttgcttcagtccctccaattcattttccagttgcttagaatagtgctcactctgttcacgtagcttcctgtctttagatgcttcagcagctagagcttctgtatgaacttccagctcttttttggctctttctgttctgcgcagttcttgccttaagctttcaactttttgcatcaccaggtccacctcttcttccttatctcggacatggcgagcaagtttctgtttttgggtgtgcaattctgttagccgctcattgatctccatgaattcctgcatggccagtttcctctgacagtgtgcgtctttcagctctttggattggttcc

SEQ ID NO: 171 - DNA, 212 bp, Homo sapiens
cgctgcagaccacactcttgatctaacactttaatattacctcagaggaatgtcacaagtagactagactttaaagagacatttgatgaagtgtctcattatgttcttgtgaatgaaagagacacatgagctgggtaatactacagtatgtggatttatattttttctatctactataccaaaagaataataattgactgatgattggttcc

SEQ ID NO: 172 - DNA, 509 bp, Homo sapiens
gggaaccaatcccccacagataccaaggaacaactgcattgtttttaaagctaaaaatgttttcagagtgagttccattttgcctaccagataatcaagatctaatataaagctttagtaattgagaaaatgtggtttggtgtaacagaacagaataatgaaaacagacctacaatacatgacaaagctgactttgtaggccagttagaagaaaacagaggagactatcactatgaccatgaggtagggaagaattttgcatataagacacaaaggatgtagttcttaaggataatcattaattaatatgactgcactaaaattaagaagctcttaaagctcttaaaggtaaaacccaaatgtccattgaaaatataggtcgggcatggtggctcacgcctgtaatcccagcactttgggaggccaaggcgggaggatcacgaggtcaggagatcgagaccatcctggctaacacgatgaaaccccatctctactaaaaatacaaaaac

SEQ ID NO: 173 - DNA, 427 bp, Homo sapiens
ctgcttgatgaccggtgaaacatttacttctcccaccatgggcgagaccttggatgtgtcctttacctctctctttgtcacttctgtcctgaactgcccatgagtgaccaggggcttccgacagaagtgtctcacaatccaaccacacagcagaaaggcattttcctttggatttgagctttgtccctgcactgactttttgtgtcatctcctcttcattcaatgtgagatactcaccttgattcatagaatgttttctcattccaggtttgtatttttttaatcaccaaaagaagcacttatcataaaagactgtgatgatgtataactgtgtatttagtttgaaaatcttcaccttatagaagcaataaacaatatttaggtgtattatcctgtgaaatttttataaatataagactttattacg

SEQ ID NO: 174 - DNA, 326 bp, Homo sapiens
cgtacaggataatgggttggctgggtatatatttaataattctatatgcaggctcaaggcattttcccttaaccctctgaagacattattccataggcttctgttgtttctgcctctttgtgctcttctcttcctcttcagagttctgcaattgcaatgttaggtcttctttttatttaacctgcttggtactcactgtgtttcctgaatttgaggtttcatgttcttcaacaattcaagaaaaaatcccagccattacccctcaaatattatctttccccaattctatctcttctctcattttgtaatttggatgcaatgcgatc

SEQ ID NO: 175 - DNA, 415 bp, Homo sapiens
gatcgcattgcataccttcggggtacaagtcagtttctacactgggtgctgatccttgcacgcgccctttctaccatctcacggtggggatggccgcagggctgtgcacccagaagaagatggctgttagcgtttcggccttcataatggcctgagactttctttctgtaggtggtctggagctgtccggctggtggccccctattttgccatttagcgaacaaccacaggaattttaaaaacaaaaacatcccaagattttttcatttcaaaatgcttcaaagtccacattagatcagatactccgctgtcggcacattcagctgaggttcattacaatcgagactgcaatgtgatctatgtttcatcttgtttttataataaaaagcttcagggaacaagcccaaagccctcg

SEQ ID NO: 176 - DNA, 567 bp, Homo sapiens
  variation 4: n is a or c or g or t.
cggnaggagtcttactctttctcccaggctggagtgcagtggcatgataccagctcactgcaatctccacctcccaggttcaagtgattctcctgcctcagcctcctgagtagctgggatcacaggtgcctgccaccacgcccggctaatttttgtatttttagtagagacagagtttcaccatgttggcaaggctggtctcaaacttctgacctcaagcagttcgcccgcttcagcctcccaaagtgctgggattacaggagtgagccaccgcacctggcctgtaccattatttgatcaacatctgtcttcccccctagactggaagctccaggtgggcagggaccatgtttgtcctcaccaaggtatgcaaggcctggacacggtaggtactttgtaaatatttgttgactagatggatggacagaaaatgaatggatggatgggccctaagctaagttctgtacattttttaaaaaaaatcagatttccaccccactaatgactaaacttgggactgtagtttctgtgtccttctgttttgcccctgcacatggcaatgcgatc

SEQ ID NO: 177 - DNA, 574 bp, Homo sapiens
cggattttaaaggaaattaaaacattttcataggcccctaagtgttatgggcttggacactgtaccctgcccaggtaagatcactcgtgggtaagaacatgaggttctcacccgtaaggcaggatttttatagaaggaaggtaggtctttcaacctatgtcctccttctgttccacaaagtggaaagccacaagccctacaaaagccttgcaagtcccagaggctgcagccgtatttattcttcaggccaagactctcaggacagagagcacccatgcaccccgcaggctgcaggccatctccctgcatttgggactgtcctgaggatggcggcttcatttttgtccctcctacctctgagtactccccattccttatgtgaagctgagccttaacccgcacccactgtattagtccgttttcaggcagctgataaagacacacccaagactgggcaatttacaaaagaaagaggtttaatggactcacagttccacatggctggggaggcctcacaatcatggtggaaggctaaaggcacgtctcacagggtggcagataagacaatgcgatc

SEQ ID NO: 178 - DNA, 283 bp, Homo sapiens
gatcaagtcccactatcaaggataagccagtaagtgtggtttagcataaaatagcttcttgcttttgcctccattcatctgttatatttgaaaaacccatagacaacttgatgtgtttggggtacttaattgacatgtttgtaagcagactaagtagtcttgttgtgaagttcttaacattgacattctgtgtatttgcacatctcaatttgacatcgaatttttggttagttgaaatataattggtaaatctttatttttaaaagatttcacggctgggcat

SEQ ID NO: 179 - DNA, 596 bp, Homo sapiens
caaacgtcggagataaaggtgtagatgcaccagctgaacgtcttgctctgtttcccattttacgtataagatgggtgatggcatgctgcattcagatccccatgggtgtatttgggatttggggtgggggattgtgtgtagaaggcaggaaagattgcccagcaactctcatttgaattgagggtggaagcggttctcgggcacagtatgccaaggcccagggcgtccaagggacagagcagagatcacagatgctgaacgggtggggaaggagcactaggcagggcatttctgatgagactttgatttgggaggtacgaaagttgtatgaggaatcttgataaactgcttagagtgaatggagatgagtaggaatcaccaccattcacatactggctctgcaagccactcagcacagctctgcacctgctcttatggaggagaagacaagcagaagagaatgttctgatctagaagagttcgtgtaagagcatgaaggcaacagcacttgagatgtggtgcacatgagcaaaatttcagtttatgtggaatttaagattaacagattaaaaggtaaaattctctccctggtaccc

SEQ ID NO: 180 - DNA, 861 bp, Homo sapiens
gggcttatcagatgcattgtggtgatgaactcatcctgctctctggaggactgcctttcatacaccacattgccttgtacaactgctctgcatttgagaatcatacttaaatgtttacgtttcaattgcctgaaagcagggccagtgtcttatgtcacctctgaccttttacggaaggggacaagtgaggtagtagctcacacctgtaatcccagtgctttgggaggccgaggtgggaggatcatttgaggccaggagttcaaagaccagcacctttgacctgaagaccctctctcctgatcatccagcaatccaggctgttcggctcttgggctcctgagtattctgcactctttacctttttttttaatcagcacagagaaaggcaattaccctcacagtacccgatgaaataagtatttttaggttggagtgattacttagtgagttccctgtaatcaaaactggtttctttttttaaggaagattcacatttctcaatctattttcctttcaaaagagagaaattagcactttatgttttaagtatacacattctgcctttctcccttcactttaaaaaaattcaagtcctggcccggatgcagtggctaacacctgtaatcccaacattttgggaggccaaggcgggcggttcacgaggtcaggagatggagaccatcttcgccaactggtaaaatttcatctctactaaaaatacaaaaattagctagacatggtggcgctcacctggaatcccagctactcaggaggctgaggcaggagagtcgcttgaacccgggagtcagaggttgcagtgagctgagatcatgtgtggcaacagagcaagactccggtctc

SEQ ID NO: 181 - DNA, 289 bp, Homo sapiens
cgaaatttgaagtgtctgcattataatttactggttcagcatccctaaatccagaaaccaaaatctgaaatgccctaataagcatctcctttaaatgctgccagcactcaagaagtttcagattttgaagctcactggatttcattttttggatttgggatgctcaacctacatatcacaacttgctaattttccagaaaccacttaccagcttggaaaaaattcttttatatctgtcatttcttttttcccttctgggaaaagagtgaaatttctatggtctgagatc

SEQ ID NO: 182 - DNA, 363 bp, Homo sapiens
gatcaagtccacaaggtcatggatgccagggaaaaacttccccctgaaaggcgcacaagcaggcggaacccagccaataatgtaaatacattaggatgcaaccagaaggtacattgcaaacccactgcttgaaaggtaataaattcatattgagctaataatgtgttaggtgaattgataatgaatttatattaagtgaatcaattaagtaattctgaattggcttttgaaagtctgcgagaggctgggcaccatggctcacacctgtaatcctagcactttgggaggccaaggccagagggttgcttgagctcaggaatttgagaccagcctgggcaacatggcaaaactccatctccaccg

SEQ ID NO: 183 - DNA, 752 bp, Homo sapiens
cgactctcttccgcaacctccagtcctccccacaacatacctagccaagtacacttacatgaactaacttagataatacgaaccacttaaacccagaggagaaactcgtccatcttcattacctgaattatctggcatgttgatattctgttacttatggagagcagactaggtgtgaaaagcacaccccagcaacctcagacagatagtcttaagtcattgcctgggattgttaactaccttctagatagttctagatagttccttctagcaggaaggggagttcagcctgagggatcaggtttcagtaagttcttatgtaatactctctccttggtatgtaaagatggtgtcactcaaccaataccagcacacatactttaagtgattttctactaatggcagtgagtttaattaaacatccttccacatgcctgcctcgtccctcttgtagcagtcaggatgctctacggggctggctgggttagtgccttgagacagtgtggacataggagtggttcatgctggtgtcgtgagggtccaatctcctcttctgcaccacccaggcaaaccaggtgtctggacaagatgggtttcagtgtttccctaatctgactttgctgggatgacccttgactcctgaaaccatagaatttcttgtagaagcaaatttttatggtcgtcattcaatgacaagaaatgacctttcaggaaagaatctgatataattggcttttcatacctgcgaaaac

SEQ ID NO: 184 - DNA, 282 bp, Homo sapiens
gctcctcttcttttatcttgtctcttttcatctgaatactgtgatgctgacgagagtatcttatgctttcttcagcctgttgcaatctgaaccgatgattttctgtgcactgatcctttctactccggagagaggctcttttggcacagatcctgccccgtttagactccagctgctggcactgccttctgagttctttcacttccaaattcttatcatcctgcagccccaccacacagtcaatgactttaagttcctctggattttcacatggatcgcaac

SEQ ID NO: 185 - DNA, 303 bp, Homo sapiens
gatcaagtcccagtacactcattagttaagtgtgacggtggtcaagtgatgttgctttgtgtgtctgttttctcttaggtggttcaggtaccctgaaataaatcctaacttatatgtctgttgctcattcatctctgtgtgtggagaaagtgatttaaagtgtcctttctttaagaaggaaagagccagggcatagtggctcgtatctgtaatcctagcactttgggaggctgaggcaggaggattgcttcagtcaggaggttgagactggcctgggcaacatagtgaaaccatgtctctacg

SEQ ID NO: 186 - DNA, 883 bp, Homo sapiens
tcgatacaggcacaaaaggaagtcagctcaacccaatcttcatggaatagggaagtgtagtaagggggaaatgaaggaagacttcctggagaagagaaatcctgaattgagtttgcagaagagataaagcatttgtcaggaagataagtagaaaaagagtgttccaggagaggagataggattttcaaagacattgagatgagagaacaaggtaagtttaaggaaatacatgaggttcaattcacctgagactcaagacagcaaataaacttctgaaagatgagattagacaaataggcaaagtttagataatggagaatctttcatgagtttgaactttatttatttatgaatgctttagtgttttttttttcaaaatattttaagaagtgaggagtgagtggtacaatcagatacaaactgattataaatgtttctggcaacactgttgagaagggaattcagtagagtgtgactggaggaagggagatgggttagggagtagtccaggaagtaatccaagggagagcaataaacaacgatgggcatggacaaggagaagatgcatattgatggactgttaagagtagtagaactaatttaatgaccaagttaatgtgaaaactgcaaaagaggaagtccagtaaaacttctgtttccagtatgagaaagatttgatggcaccactcactaagatacaggtagtgcacagattagagatgagcagagaagagagatctgctgcaaatatgttaaatgcatccacttggagaggtccgatgggtggctgcttatggacattgtggagttcaggcgagagatttaaattagagatcttcagctctaatctttgtgaatccttaagaatatgtctaacttaccc

SEQ ID NO: 187 - DNA, 260 bp, Homo sapiens
gtagagatgcatttttgccatattgtccaggctggtctccaactcctgggcttaagcgatccacccacctcgacctcccaaaatgttggaattacaggtgtgagccactgcacccaacccacgcccatttttaacaaatgaagaaactgaggcttgcagaggttagatcatttgcccaaaatcacatgctggatccagaactagagcccccagtcttgttattctcaacactttttcctccacactacagggacttgatc

SEQ ID NO: 188 - DNA, 964 bp, Homo sapiens
gatctgactgcactaaatagacagtttttaatctcaattggacatctgaagatttctcaagctgttgttggcttcagcagaaacaaatttttctgctttgttgattaaaccagtggtctccaccattgcttatcggcctgcaacgagctgttctttttaattaaaagtgagccaccacgagagagcaattaacagctagtaattactgtgctcttcagaacggcaacattttgcctttcattccaattatataatggacaagtgggtttttgatcttcaaattagtgagctcttgtcctccttgtttggaggcagaattggcaggttaatgatggcctgtgggggcactctttcctctgactgatgtgaggcaagaacgccttaaaggaggcagtgagagcctttcaagacccgcagttccacctttatgataaagacagagaaagagtctttattttcagagggttttgatgaggaggaaagataggcttacatgtggcctccaaacagtgccatgtgcttttccttcttcttatcctccagcaaaaggtgaccaagtttcaaaacatggaaaactgaaagtttcagatgtgcaagatggctaagttctggagatccagtggacagactgatgactatagttagcaatattgtattgcatacttgaaattttcttaagaatgtagatcttaaatgttctcaccatgtgtgtgcacgtatgcatacacacaaacacacactcacacaagattactatgttaggtgattatatatgtcaactagcttgattgtggtaatcatttcacaatatatatgtatatcaagttatgtacctcaaatatatacagtttctatttgcaaatgtacaccaataaagctgaaataaacccaaggaaaactggaattagattatgtgatgttttggtatgcctttggtaagggcataaaataccacatgttggcg

SEQ ID NO: 189 - DNA, 516 bp, Homo sapiens
  variation 509: n is a or c or g or t.
gggacttgggtgtaactggatcagattttctggaattcaagggagaagccgagatacttccctcacagaaattgttaatatcaatgcttagctttcttgccagttcctcatcacttttcagttgttcttccatcgctcttcgccttttttctgcctgtcttttttcctcttcttcctcctctgccaacaacctctgtatgtattcttcactggctttgttttcttcttcctcgctggcccgtcgctctgccgccaccttgcttatttcctcttcatattctcttctcagttccccaggtttactgagcagacgaactggctgatagtcatcagccacttcctctgattcttggccagacgctctaagcttgcactccctgggatagtgtttttgaattatcgtccacagttccacgttgacgaagagaatttcttcgggtatggtacccgagtccacgacgatacccggcggcgacagaagggacagcataaactcgccttttcgacgngtcacct

SEQ ID NO: 190 - DNA, 297 bp, Homo sapiens
cgttctcaggttccccctttggctgctctgcctcactcgaagtagcaggtggagtttcattctcttgttcttctacagtggagacagattcctctgaacttatgtctggttctggcttttcttcctccccttcagcaagcttgcttttgggaggagtttcccgggtagaattcacagttcgacgaatcggcatggtgctatcttctaccttctaagaaaagagatggccataatcaaacatgcattaaaaaattctcttttctctctttctcaaagggcctaactgggtgtcagatc

SEQ ID NO: 191 - DNA, 527 bp, Homo sapiens
gatcaagtcccattatagagaatgggttatatggtatttgcatataacctacatacatcctcctgtgtactttaaatcatctgaagatgattacttatacctaataaaatacctacacatcacttcatttgcatggattcaacatagtgcttggtgttcagtaaattcaagttttgcttttttggaactttgtggaatttttctttttctgaatatttttgattggcagttggttgaagccatggatgcagaacccatggatatggagggctgactgtacttacttacatatctatctatctttgagtaacaaactctttgggggaaaagagggaagtgtattgagcaagagaaggcaaaaaacattacataatattttgattctgttaccgtgtgtctatagattgtttcattctaatcaagacctaagaaggtatgattctgtgactgatttagaattctggggtggaaatatcctggaaccatgttttttaataaattctgtctcttggaaagagttaacaacg

SEQ ID NO: 192 - DNA, 525 bp, Homo sapiens
gatcaagtccccttatataaaatggcatagtatttgcatgtaacctatgcacatcctctcatatactttaaatcatctctaggtgttcttacagtacctaatacaatgtaaatgttatgcaatagttgtcatactacattgggtttttttgttattattttttatagtttttcccccaaatcttttcaatcggcagttggtagaatccacaaatggagaacccatggataagcagggttgactgtattaaagtgtttctaacgattttcaaatcaaacattctacggtgatttatttatgccttgacatcaccaggtgatgaaagtctctcacacttctctaccaaattatctcgtaaaggcaaaagaaaggaaaaaaaccccataatctaggaggtggtctaaccataaaaagtaacatttctataaattgtatcttgagagttcaaggaaatttgaattccctctattccataatgtatttaaacataaaaatatttacctcttccccaccttcttcccaccg

SEQ ID NO: 193 - DNA, 507 bp, Homo sapiens
cgtttgggaagttatttatttctgaaagtgtcatttatatagtttggatgtcccctccaactctcatgttgagatgtaatacccagtgttgggggtggggcctaatgggaggtgtttgggtcatgggagtgggtatccctcgtggcttggtgttgtcctaaaggtagtgagtgaggctctcagaagatctggtggtttaaagtgtgttcacttcctccattcttgctcttccttctgttttggccatgtgatacgcttgctttcccttcacattcaagaagaaaaagtttaattgcttcaaagagaataaaatactcaggaatccaacttacaagggacgtgaaggacctcttcaaggagaactacaaaccactgctcagtgaaataaaataggatacaaacaaatggaagaacattccatgctcatgagtaggaagaatcaatatcgtgaaaatggccatactgcccaaggtaatttatagattcaatgccatccccatcaagcag

SEQ ID NO: 194 - DNA, 509 bp, Homo sapiens
ctgcttgatgagaagaaagatgtgtgtggcatcccctgagatggtttccttctcgtttctacaaagtagataggccttggtagaaactgctgtttctcagtagttggaccattttatagccagattcttcccactctggaatttggctacaaaaggtagcaaattgtttaccaaggtttgtagcagtatcgcttctcaccagagtgaataaaaaatgaaagctgtggccgaacatggtggcttgcgcctgtgatcccagcactttgggaggccaaggtgggaggattgcttgaggccaggaattcaaaaccagcctgggcaacatagtaagaccccatctctacaaaaaatgaaaaaaattagccgggtatggtggcgcacacctgtggtcccagctactcaggagactaaggtgagaggattgtttgagcccagaagttcaaggctgcagtgagctatgatagtgccactgtactccagcctgtgtgactgagcaagaacctgtctcg

SEQ ID NO: 195 - DNA, 501 bp, Homo sapiens
cgatcatgagaatcatttttcactttttttttttttggagacagagtcttactctgtcatccagggtggagtaaagtggcgtgatcttggctcactgcaacctctgcctcccaggttcaagcaattttcgcgcctcagcctcccccgtagttcagtagttgggattacaggtgcgcacgaccacccccggctaatttttatttatttatttattttgcatttttagtagagacagggctttgccatgttgcccgggctggtctcgaactcctgagctcaagtgatctacccgcctcagcctcccaaagtgctagaactacaggcatgaaccaccgcacccggcccatttttcacttcttaagtttttttccctgaggtttcctatctcttcattagggaaacaatagagcccaacaggtcagcctacagacactgttgacaaaaaggactttagttcaagtcctagttctgccacttgttagtatatatgacatcaagcag

SEQ ID NO: 196 - DNA, 497 bp, Homo sapiens
  variation 3: n is a or c or g or t.
ggnaggattacacaaggtttaaatgagataccacatgtcaaaacacttagtatatagcacataattctaaatagtatttaccataattagccattattggccgaggacacacaaggaatacacgattaagctgaaatagtaatacaaacctaggtctattttttggccctaaagacttcccctttttgtgtctgtttcatgcacatctgattagtgaactgaagcctcatggccagcccagcttcaggtgggtatagacaccgagcccccacataataataagctgagcctaataccttccccagggatgttcttttgatctcctggtttcctgacaatttcggatcgatgattaatggttagtttacttgatccatggtccaatttcttcaaagtgtcatacgctaaataactgtatgaaccaaatagttctttttacttcagtaactttgtagtcactatgtgtattttaacaatgtaactgtaccatcaagcag

SEQ ID NO: 197 - DNA, 413 bp, Homo sapiens
cgatttctaaactttatggaaaaagccaatgacaatcaattagccaaaaacgagacgccaaatcccatctcactccagcgccatgccctcttctcaaacgctcgccggctctaggacctctccaccagcacagttctcaggagtcctgctggtctcccagtgctgttctaagggtccacatgaaggcaggtcaggcgggactccccgggcaagcacttctgtacgcggaaagcacgaggcgcaggagccatggagagccggtcctttggagacaggaatacttaagaggattggcaaaccggagaaacgctgggagaggtgccccgcactgcggtggtaaggcagagtctcagttctcctcgggcttgtcggctggcggtccacaaggctgaagcatcttttccatcaagcag

SEQ ID NO: 198 - DNA, 411 bp, Homo sapiens
ctgcttgatgatggtaataactaacatgtaaatctttaataaacagtgtacattcttaatctgtccatcaactcggtacagtcagtattgttgtccgtatttcacagattaagacaccagcctagaggccgagtgcagtggctcacgcctgtaatcccagcactttgggaggcccaggcgggtggatcaccttaggtcaggagtttaggataagcctggccatcatggtgaaaccccacctctactaaaaatataaaattagccaggtgtggtggcacgcacctgtaatcccagctgctcgggaggctgagagaggagaatcagttgaaccagggaggctgaggttgcagtgagctgagatcacacaccactgcactccagcctgggcgacaagagtaaaactccatctcg

SEQ ID NO: 199 - DNA, 893 bp, Homo sapiens
gggcgaggcagtctcactctgtcgccaggctggaatgcagtggcacaatctcagctcaccacaacctcacgattctcctggctcagcctcccgagtagctgggactacaggcgtgtgccaccatgcccagctaatttttgcatttttagcagagacggggtttcaccatattggcaaggatggtctcgatctcttgaccttgtgatccgcccgccttggcctcccaaagtgctgggataacaggcgtgggccactgtacctggccttttttttttttgaaacggagtcttattctgtcatccaggctgcagtgtggcaatctcgggctcactgcaacctccccctcctgggttcaagcgattctcctgcctcagcctcctgagaagctgagactacaggcacgtgccattaggcccagctaatttttgtatttttagtagagacggggttttgccatgttggccagggtggtcttgaattcatgatctcaagtgattcgtccaccttggcccaaagtgctgggattacaggtgtgagccactgtgtccagccaagacttcctaaacttttatgctacaataccctactgactcttcagggaaagggcagcacaaatgtgatgagttcctggccataggtcttatacttctccaacctcacgtaccctaagaaatctgtctgacaaattgattgccattaccatccttcaccatcacatcctaagataactacttccaaaaggtagttcaggatttaactactaccagtcttaggacaagtacaagttcagcaagaggttataatgagaccttatccatggcagagacaaagtgaagaagaatgtaattcaggcaatgcagggtgagtggacctttatcttccccatcaagcag

SEQ ID NO: 200 - DNA, 858 bp, Homo sapiens
ctgcttgatgtcagtttaacaattttatgaaccagtttctttaatagtgttgtggaaactcttagccagagcaatggtataatcttaaagttataagaaacctgtacttgttcagagtctcttccatgaatctccttgaagacgaaacactttaggattatagttgcatgcaaaagctttcaggaaagcatcagagtaaaaattatctctggatgacaggacttaaaatggctatgataagaatttattatgcaatcgacaaggaaattttattttttgtgacatcagtattttaacataaaaatcaaaattatgactggtaacatcataacaggacatatcagatttgtaggaatttcataccatttctagaacactcaataacatacccaggcaaatataactcaaggaaagttaaacatcattgcttatttgacagtgcttaccgtgtaaatttaacatactgaataaggtagtttaacatctctctttttatgaggagagagaacagattcttttgagatattctaggaatcccctggaaactaccgaagttagttcaaggtgagaaagacctaatggaggctgggcatggtggctcatacctgtaatcccagcattttgggaggccaaggcaggcagatgtttgctgaggcaggaagcagcctgaccaacatggtgaaaccctgtctctactaaattaaaaaattagctggacgtggtggtgcatgcctgtggtcccggttactcagaaggctgaggcaggaaaatcgctcgaacctgggagatggaggttgtagtgaaccgaaatcgcaccattgcattcaagcctgggcaacagagccagactccgtctcg

SEQ ID NO: 201 - DNA, 780 bp, Homo sapiens
cgtagagacggggtttcaccatgttggccaggctggtctctaactccagagctcaagtgatccacctacctcggcctcccaaagtgctaggattataggcatgagccaccgggcccggcctcagcttgcatttttatattaaaataacctcacaggctgcttttagttagcaacagcttgggtatttaatagtgtgttttcaaattatgagtagtgttacccattttgctcttaaaataggctcagcgctgactctgaaactctgtgccttggagtagcatggtgttttgtgcactcatcccggaaataaatgttctaaacacagagtcaaagtcgcaaagaaacaaagaggaggctgtggacaagggaggccaagcagcctgtattcctacataaccattaaagaacaatttgcgtggctatataaaatctaattttaaaaaatatttagaaattttaagacaaccatttcaaaaaaacatttaaaaaaggcattttccttttcttgagtaaggggagtcctttcaggcctttgttttaaatttgtcaaagcaacccatgttcatagtaaagtaagaattcatgtgcaacacaaagcgaaaattccgaatcctgtatgcgccccacccctagtataattactattagtggtttcttgtgcttacaacaagaaaaacgcctttcttaatagaagagggcttgcctggtaagggggcccatgggtcttctggcgttggggatgtacaggttatgttcatgccatgtcaactcatcaagcag

SEQ ID NO: 202 - DNA, 560 bp, Homo sapiens
ctgcttgatggcaaaaggggagggtggagggtgggagaggaaatgtcagggcatgtgctctgataaaataaaggcagggaaacaagctgaattacttgaaattacttgaattttcttgtcttaaaactgaaaaaaaaatgtagttacacgttaaaatctgcaaatggtttttacacctctgattttaacatgaacttatactaatgtttggaatcttatgtcagaaatataagcagctatgtacttagaataggttttgaatgggagaggtagaacagagagagaattaagaagggatctgacttataaaagactagaatgtgattagagtgatagaacataccaatgttaccaagaaattgacaagctgctggctttaagcttatgcaagtggtagttgggaaagtaggaggtgtggaagagggtttgcattttggattaattcatgcaaaatgaaggaggaagcctggtctaagaagatactgtctttcaatagaaatgatttctaaactgctacagattaagaatagataatctgattgctgttgttttgtttgttc

SEQ ID NO: 203 - DNA, 877 bp, Homo sapiens
ctgcttgatggtattgaaaggtgactataatgagggaagaaaggaggaggtaaagagagaagaatttgtcccagatctgtttaaagtttcaaaatttaaaaagggacccattaaattatgggaaaatggctatagagtgtgagcctcctttgaccatatgctcaaagaccgtactctgccacctgccttccaggtagctattctagaaactcagtcctttgtggaaacccaactaccttttaaaagtctctttccagattccaaaaggacaagagatcagagagtcacatatacgcctcttgttttattttcttgctttcaccggtattattgccaagaaaatcgtagggaaaaactttaaacttttcttttcagttgatccctttgacatcacctctcatgtttaaaatcaggaaaacacacccctaaaatttgcactctcttccgttttgaaaaagaaaacccacacacaaatgcacactattaccgtctttcaccctgcgctatatttccaaagtgtattataatccagatattgccccatctcaaacatgttaagtcagactgtgctgaaagactttccagggacggtcaacagggtatatgttcagtggctgccctgaaatcctggtggggatgaggatcacgcttcatcatcaaaggggatgcccatcccctgataagctcccagtccttttggaagatttctttgaatgttaattgcattttcagttttgctcatttcccaccccaatgttttgtctgcaacatcgcttacactggattctttctatttttattcctatcattaaatggtagtgctgtaaattctgcattctgcaattaatgttaaataaactgctttaattcattgacg

SEQ ID NO: 204 - DNA, 486 bp, Homo sapiens
cggagacagagtttcactcttgttgcccaggctggaatgcaatggtgcgatttcgactcacggtaacctctgcctcccaggttcaagcgattctcctgcctcaaactcctgagtagctgggattacaggcatgcaccaccatgcccggctaattttgtatttttagtagagacgaggtttctctatgttggtcagactggtctcgaactcctgacctccggtgatctgcctgcctcggcctcccaaagcgctgggattacgggcttgagccactgagcccaacgctgtttgtgattgttaaaggaggtacatcctgctctttcagtattatattttcttaattgctcttaaactttctcttatttttgccataaatgaaaacaaaatgcattattactattcatatcatacaccctcctgtatcctaggtgacaggtcaaaaagtgactcacacacacattattttctagtattggcatcaagcag

SEQ ID NO: 205 - DNA, 397 bp, Homo sapiens
cgaattaaagagatgaatttattggtaaatagataaaattaacacctattttaatatatccaaaccccttccttatatatattaggtaaattaaaagaaaaaaattatcaaagcaatactacagccagctacatcgccaatttacaaatgagttagtaagtaccataagtttgtttgaatatcaggtgcttcagagtttttctcaaacagttacaaaagagattaggttcccaatcagttcacaaaagctaatttaaagaatgtagcttaactacagtactgaggttgtcacacacttaactttcggtctcttgcttatttattcatatctgaggttcactgtttctactaggatacattccgcccacacccacaccttggtccccacatcaagcag

SEQ ID NO: 206 - DNA, 397 bp, Homo sapiens
ctgcttgatgttaatcatactgggactaagaggatagagaatggtaggagctgggatacccctaaacattcacattaaaacaaaaaaaacccaaagctaaaaaacaactgggcaggagctaaataaaaatctaattttgagaggctgtatctggctcaggcctcctactttgtaacccatggaatatgtgaaagcatttgaaaaactatagcactgatctcacatgggcagacacactctcagagagatgtggtgggagccatggcgcagtctgcctaggcagtggcaggagcgcagaagactctgattcctctcctcggtcctaagaccgaatgtgtgtcaggacatgtggtcagggaagagaagctatttaactgaaccagtaatagtagcaccg

SEQ ID NO: 207 - DNA, 652 bp, Homo sapiens
  variation 645: n is a or c or g or t.
cgtctattatttctttatgcttactctgccatttattaaaggagggaaaatgtgaacgatgaaaatatttacttcaaattttgactatttaaactttggaaaacttttcccggagaatgactttcttggttgaggtcaatttcctgaaggactgagatgaggttaattatgaggcacacagtgaagtcttaatggcttccttaagttcacacctatgaattcctggagtggccagagaacatgttactactccagggtcaatggtggaagcatgttgatggaagcatgctgatgaaaacaagaaagacttgtggaaatgcttagcttgctcctcaaaatggaaaaaaattcaattctgtcccaatcatactggaacaatacatatgagacttcaattgtagaaggtcaacaacttcaggtaaataaccatgaagtcaacaggatagtcaccataacctcacataatccattaactgtaatacactattcaccaggtctttggtttcagtgaagaaagtggggcttgtagcaaaggacccagatacaaacacaaatagggaactaaaatggttcttcaaagacatgcattagagtgcacatcagagtgctaaaccaccaagtccttttaatttgcaagtggacatnaaagcag

SEQ ID NO: 208 - DNA, 659 bp, Homo sapiens
  variation 6, 648: n is a or c or g or t.
ctgctnaatgggattggggccaaggagtcagtgcatcgtggttggtatggaagatggaatgggaggcagagcagcttctcctgttccttagcctgcgatgtttcagctgttaggaagcaattcaggcaggaaagcaagtctgctccatttaggttagtcccacatttgctgtttgtttgactttgagcaggacacagaagtccttcaagccccacctctccccctgtgtaatgggcagattttactcgctcatccctaactctccttagcactccagaggcacaggcataaggtacaagttttgcagctggaaatgcataaatgtggatcccagttccagcactttctggctgagtagtcttggatctatcagctcacttctttgagtatcagcttcttcctctgctatgtcctggtttccatgttttccttgcagagctgcaataagggtcagagatgctatagaatgggtcatagtgcctagcaggctggtaaccaatgctagccatattttcagtctaaagattgggcaaatgttggtctgtccttggttgaccaacttcttcctttgagaagcacatagcatctgtacacacagaagcgtgtgcatgtgagggattcatacatatctaagaaccttcttgcaanggaagtttacg

SEQ ID NO: 209 - DNA, 564 bp, Homo sapiens
cgtgttctccaggtgattttaggtaggaaatcagggttgagagtcactgctttaaaggaagaaaattaaggagcagtaccttagaagaggatattaaacaggggtggagtttgctctaggacagagtagaacatattcttgttgctctgagatacaaggaaacctgactagatacatttaagagatctggttgagatacagtggctctttgctcatagtaaaaaagtattaatgaaatattttaaaattgggacttccagcaactcagaattagttggtctatgaaaaagtaaacacaaggaagacctagaaaatcagtatatagaagttaagcagtgaaactaaaaggtttctttttaaatgaatgaatttgtgttgatatacttgtcttttcttataaccattattaaagcaattatgaaggctttatttttaaaacataaggtaaaacaactgagctaaaatcatgacaactctatcagcatttctttatccacgaatacacatcaccaaaaaatggcactactgatcgtccgtccaggggaaataattagcatcaagcag

SEQ ID NO: 210 - DNA, 337 bp, Homo sapiens
gatccagtacccagtaaagtgctctcacatgtgttgatagctggacatcttccataatgcttctttcactaaaacaaactccttgcgaatttaacccatacatttgggttaaattctacataaaatcatggaatttaagagctggaagagggttggcaataacctaatctagtctctgttttaacaaagagaaaactaaattttaaagatggtaagtggtcaggcgcggtgactcatgcctgtaatcccagaactttgagaggccaaggtgagtggatcacttgaggtcaggagttcgagaccagcttggccaacatgatgaaaccccatctctacg

SEQ ID NO: 211 - DNA, 970 bp, Homo sapiens
cggtgaaaggaattgaattagaaagtcaacaatctagaaacttcaaaattactgcagccacctactactttccaaaaataagcacccatcaaaactagactctatcatttgttacccatgcgtgttctattactaatgttctcaaatcaaatttgagcttaaaaacagaaaatctacagtttcgttgacgacccctcccacccccaactattccacccttcagccccatggcatgttaaagggaagaacaatgaattctggttgttacaagagaatatttggagcatctgactctttttgctgtcaaccagggtatcaacttgaggcatccataatgactcgactccctgtttgattccttctctccttttattctctagaaagcccttcctggaccctcttgttggactaaatgacctttctccacattcctaaccaccctgtgcaaacctctctttgagcacctgccacattctgttgaaattaagtcttatcctcccactgtattttaaagtctttgaggacgatgcccatttgcaagtcaccgttgtatgcttggtgcctggcacaaagcaggtgatctgaaatttctggtggaagtggaagggtcatgtgtatgttgggcgggtgtgtgtgagaaggtaaaggacacgtttttcatttgcctttatatagcgaccactcatcttaacttcaacccctattccaaatcttcctctgaattgtacaaatttttaattgttacattctctgtagatgggtttggatgacacaggaaaccccttaaagtcatctgcaatgttctcagccttgagtttgatggattacttctgtgagtgatggtgagtcgtggcagtagacagctcatccaatcccttgagttactgttacttgagactttttcgctttcaagaaaaaggaacatgagctaacataagaaaatgcgaaagtcttatatgggtactggatc

SEQ ID NO: 212 - DNA, 317 bp, Homo sapiens
gatccagtacggtcaagcaggcaagatcagattcacaggtaaaacttgcttgatggcacttcaacgtttcttcctatttcctgagtggtattgatgtgtgagagatagaatgttaaatggtgaaagaaactttataagctagtagttcctttttttggtagtagaaccccagtatatagtacaaaaataaaaaatgtggatggttgcagtggctcatacctgtattcctggcactttgggaggccaaggcaggcagatcacttgaggtcaagagtttgagaccagcctggccaacatggtgaaatcctgtctctacg

SEQ ID NO: 213 - DNA, 763 bp, Homo sapiens
cgatggcagtatttttaatcttgaaaagaaaaatacaacacagctcaaggaaggaagaatattctaagacacaccaaatctagtacttctctgtgtatgaccaagaaactgttttctgagagtggcttagaagaaaatccatttactagaaccctttcttagaggcagtcatgtgatccacatttgctacctctgacctagaggatactgtgtaactgggaacaaaaacttatcagaagggtttcaaaactcttttgatattgctattttactccctagagtttagcacttgcaaaaaatgttagtgattacagagtcagagttgaaaatatatctcacaaggaaagagacatccagccaagaaaacacaaatatttaaaccatactgaaaaaaagcataacatgttgagattattaccacaagataaaagcccttctttatagcccacagtataggagaaatcaacaaattctcttggggaaattatattccaaagctgaccagcagtagtgtaacgcatcacacagcaattctcttcaaagttctccagaatatccaaagaagtcatcaagctgtcccaatccaaacgacaaggccctgggcgtatatggtctattatactatagacaaggtcatctataacacccttgggctttgtagagatatccattaaattcttctgagggttttctccaaacagttacatctttcgttttcttagcaactcgccacttatcttcttcaatgctatggtactggatc

SEQ ID NO: 214 - DNA, 518 bp, Homo sapiens
gatccagtaccagcgacagactttgcaaacatgcagatggttctcacatgtcttccttgtctcattttcagggcacgtgtcctaggttctttcgattacgtctctcaaggcaaggtttccagatctctctgtatcctcatgcttcccttttggatgcaccttaattttaaaatacctctttttctcattaattagatcacttcaagttaaatacaaaacatggcaagatggatttaaatttagagggatataagtatacataagagaagaccaatctctacttttaaaaatgcagttaattaacaataaagtaaaatatagtgaaggtactatatgcttatttgaactttgctgttgttaaggacttgcattcctctattaactttttttaaaaaactcgttttgaaaactagcattggatgtgggtttccaatgatctagaaacatgtaaactacaacataatcgtttcaagaatttaaaataattttgcagtagagaatgttaatgttttcacccg

SEQ ID NO: 215 - DNA, 334 bp, Homo sapiens
cgcttagctccatagccccataaccccattttttaactgagtcagaccctaactcaagaaggatgagttaaatgacttctaaagtttccttctaatcctttcacagtttgatttgactccttctaataaagtcagtctggactgataatgtgacccacccaatactaccctaggagtcaatagcacagatgagctaaaacctggtggtctctcggctcccagtgtgggactgtcaccaggactatattcttcacctttttcacaacccagaactgctgctgggcaagccaagaaagcagaaaaaaatcacttacttgttttggccaatgcgatc

SEQ ID NO: 216 - DNA, 304 bp, Homo sapiens
cgctgtatagctaacagcatagccaatgccctgatcttctagccacactgtaatttcttgtccttgcatcacaaccccagctgtagtcatcaaagtggaaaaactgacagcattgtctgaaagacacagcttgggaaaattgctccttgtagctcccctgtcaatgaatattatcatatttttctcatggataaggttactgtgttttttttgctgggccgggggtggggaggcttctacatacttagctactaagcacattaccattccgtttgtggaatggactaaacgtgacaatgcgatc

SEQ ID NO: 217 - DNA, 304 bp, Homo sapiens
cggcatacaggccataatttccatccatgaaaatgaaaaatggcgatgcaagctatactttgtctacaatcaccaactaggtgaattctcagtggtctctttagagtctctgaacagttaatctttgtagaaatcaatcaaaataatccatggctcgagttcagtttggccagcacaccaagagcaggtggttaaaaacattcggactcaatgcttttcacaatacaaattcagaggatgaatctttacacaaatcccactacctctattctaacagttctgacttgtttcatccaatgcgatc

SEQ ID NO: 218 - DNA, 730 bp, Homo sapiens
  variation 2, 10: n is a or c or g or t.
tnacacaggngatcgcacttgtggggaggtgaggccaggcagctgggacattctagaaatgcctttttagaattatggcatttctaaaaaggtacttccagaacagttgtttagatggaattcaaagtaaagttcagattagcttttgaggaaaaaggaatgctaccgtaaatgtgaaccttaatccagtatactccagccatcctttggggatgaataaattttcaggtttacctaagtcttaacaatttctttactagccccttaactgttactttagcaggtttgataaaatgtgctaaaaatatgcatttttgagtttttaactttagtgggaagtagatgtttcatcaacttctttggaaaataaagacactggcatataaactatatttttttgagacaggatctcactctgttgtccaagctggaatgcagtaatgcaaacatggctcactgtagcctcgacctcgtgggctcaagcaatcctcccacctcagcctcctgactagtggaaccacagacatgagctgctgcacccagctaaaatggagtatttttaatttctgggtcttttaaatgcatttggaggtctttagttttacctcactgaaattaggattttaattataaataatcaaagatgtgaaccttacagacattttaaagccattatattttttctataaaccctgttctcgtttggaggagaaagaaattggaattttcg

SEQ ID NO: 219 - DNA, 509 bp, Homo sapiens
caattgtaccatctagtttttttaaattataattaattaatttgttttttgagaccaagtctcactctgttgcccaggctggagtccagtggcatgatctcagctcactgcaacctccgcctcctgggttcaagcaattctgtgcctcagcctcccatgtagctgggattacaggtgtgagcaaccacgcctggctaatttttgtatttttagtagagaccaggtttcactatgttggccaggctggtctcgaactcctgacctcaggtaatccgttcgccttggcctcccaaagtgccgggattacaggcgtgagccacagtgcccggccaccatctagtttttaaacagaaaccacaagcatctagcccctgctggaggagaaactttaatttttccttccatatatgtgtgtgatgggtctttcctggtgacatattaagtgggtagtacttggggaaatccaaaactattaagtttggggttttgaactctggacccgacgtttg

SEQ ID NO: 220 - DNA, 487 bp, Homo sapiens
gatcgcattgccacaatccagcctggatgacagagtgagaccccatctccagaaaaaaaaaaagaaattagctggcgcgtgatggtgtggctgagggaggctcaggtgggaggattgcttgagctcaggtgtctgagactacagtgagctaggctctcctcactgcactccagcctggaagatagagcaagaccctgtctcttaaaaaacaggcaaattattgattatagttcagtgggctaaatcctgtggtagaaacattcatgcatagggtggtgcatccacatcagtcttaggtgggaggagggtcatggaaggcagatttttggttgcacttgaactgaactttgaaggatgattaggagtaagctgggtgggggaagggaagaacctaccaggcagaaggactggcataagtaaaggcttatttgtaagaagtagcatggcacattaagggaagttgaggttgttcagtatgaccctaccg

SEQ ID NO: 221 - DNA, 333 bp, Homo sapiens
cgtcacaccaacactgagaaggtagtattttcattttttctgaggctcagagtttaaatagctttcccgagatcacacattggcatgctgggatttgaacccagagagtctgaatccagagtctgaacttcgatcctctgcctcagcctctcaagtagctgggagagaatttttaaattttttgtagaaacagggtctcactacattgcccacactggtcttgaactcctggcctcaagcaatcctcccaccttggcctcccaaagggctgggattacaggggtgagtcactgtgcccagaacagagcctgaacttttaacgacaatgcgatc

SEQ ID NO: 222 - DNA, 763 bp, Homo sapiens
gatcgcattgtcacaggcttgtccttagttggagggtgatctgtgttgggaaacctttttagaagaaattactcttgagctgagatttatgccctgtttagtgtgtaatcccagaattactagtgaaatgggttggtaaatagggtaattttttttccctcctgaatttggtggatcttttagagttgaatagacagcactttctgcaacagatggggatttagacagggaggacactttagttgtgcttagtttcttctaccatccgtggcatggaaataattaggtattataatgatttaaaggacttaagttcagtctttttggttcttttcttttcataagattcttattaggtacgataaagcattgattgcaagtacttctttaaccatacatgatgatgtattttcgtgaaagtagagctgtaaaaatctagcccattcagctgggctcaggggctcatgcctgtaatcccagcactttgggaggctgaggcagaaggatcacttgagatcaggagttcaagaccagcctaagcaacatagcaagaccccatctctacagaaaatttaaaaaattagctggacatgcccctgtagtcccactcaggagactgaggtgggaggatcacttgagcccaggaggtcgaggctacagtgagctgtgattgtgccactgcactccagcctgagtgacagagcaagaccctgtctcaaaaataaaataaatctgactcattaaattgatttttaaatactcg

SEQ ID NO: 223 - DNA, 470 bp, Homo sapiens
cttgattgcccattacaacaactgggaggttagaaaatatatattgatattaagtccctccccagacagattatattagagtctccagtaggagctttctccccaggggacattattttgtagccagggctgagaatcactgcattaaggacaaatggactagggagtaaaaagctcaaaagtgggcttgcagatttagagttgtagatcacatgctgtttacctctaaatttatttaataatttgaaatatgggaaaaagataattacttagttcaaaatttaatttagataaatttccgctatagcttaaaatgataactagtctatgtctcagaataattaattttggctgggcgtggtggctcactcctgtaaatcccagcaccctgggaggccaaggtgggtggatcacttgaggtcaggagttcgagaccagcctgcccaacatggtgaaaccccgtctctacg

SEQ ID NO: 224 - DNA, 286 bp, Homo sapiens
gatctcagaccacgatggaattaaactaaaaatcaataacagaaagataactggaaagtcctcaaatatatggatatttaaaaatgtgctttcgaataacacttatgtcaaagaagaaatctcaagagaaatgtaaaaatcttgaactaaatgaaaatataacctaaaatttgtgggatgcagtgatagcagtgcttagagggaaatttataacactgaatgcttatattagaagagaagaaagatctaacatcaatgacctaagcttctaccttagaaaactacg

SEQ ID NO: 225 - DNA, 851 bp, Homo sapiens
caaacgtcggactcaggaagcctgcagtatatgttcaatgtcccctatggtgtgggcaaagctgcggtgaggacccagtggcataggagctgaggaggtaaaagacagtgatgttaccgatagctcaggaggtacaatccccagtggtcagaatgtgtgacaagctggctgctgactgtgcccacgagctgcggcgccatggggtcagctgtgtgtctctgtggccggggattgtgcagacagaactgctgaaggagcatatggcaaaggaggaggtcctgcaggatcctgtgtcgaagcagttcaaatcaagccttctcatctgcggaaaccacagaattgagtggcaaatgtgtggtggctttggcaacagatcccaatatcctgagcctgagtggtaaggtgctgccatcctgtgaccttgctcgacgctatggccttcgggatgtggacggccgccccgtccaagactatttgtctttgagctctgttctctcacacgtgtccggcctgggctggctggcctcctacctgccctccttcctccgtgtgcccaagtggattattgccctctacactagcaagttctaaccctcctggtctgacactacgtctctgcttgtcttctcatttggacttggtggtttgtcctgtctcagtgaaacagcagcctttcttgtttacccatacccttgatatgaaagagaagccctctgctgtgtgtccgtggtgagttctggggtgcgcctaggtcccttctttgtgccttggttttccttgtccttctttttacttttgccttagtattgaaaaatgctcttggagctaataaaagtctcatttctctttcg

SEQ ID NO: 226 - DNA, 158 bp, Homo sapiens
ctttctacccggcctcaacctttcccaccttctcaggaacatattcatttttcctcttcctcttctctaccctcaactgtctctctgctatgtttttccgagcggataacaatttcacacaggctttctaccccatagaataaaactgacataaaaca

SEQ ID NO: 227 - DNA, 785 bp, Homo sapiens
  variation 687, 725, 734, 769, 778: n is a or c or g or t.
ctgcttgatgtgtagggaagggagggggcctgaataatccctgaggagtagtagaatagcagatggaacactgagaagttatttccttgaggatagatttccacaatggaaaggaaatgagaggttctaagaggcgggctagtggcttgtactatagcataacctgcctttgctggtgtgtggcgattaggcctggtggaaccgccatcaataaatcaagcgtgatcagggtgaggaacaggaaagaaggaaatttggggaaatgtgaatgtcaggtggatcagagagatacggtcatgggggtcaggtgtggtatcaggaataatgtgggaggctggattgaagtatgggccaggaacaatggtaattgtgggagactcaacaaacagtgagtacagctgaaggagccgggaagcagaaagtatatgcgtcaggtatgaggaagaaaatagattttggaagttatgagaactgcagagagtgagttgaacacagtttgtgattttgagggcctctaaaagtattaaagcagtggcagctgctgcacgcagacatgagggctaggctaaaacagtaaggtcaagttgtttggacagaaaggctacagggtgtggtcctggctcttgtgtaagaattctgacccgagctaaccatgcttaggatggaaaggagttgtgggttttgtanaaagtgctggggttttgagaagatcagtttggaccacnaattggcanggaagagcccatgtgttttttaatgagaattattncccagatanggtaccg

SEQ ID NO: 228 - DNA, 156 bp, Homo sapiens
gatcaagtccctgatacaaaatggcatgatgtttgcatttatactgtgcacatcctcctgcatgctttaaattctgtctctagattatagtacttaatacaatgtaagtgctatgtacatagttgccatactgtgttgtttagggaataatggcac

SEQ ID NO: 229 - DNA, 541 bp, Homo sapiens
ctgcttgatgccataaccaccagccacatgttggctgtttcaatttaaattaatgaagcaagataaaattaaaaatttacttcctcaataacagtagccacattttgaattttcagtggccctgtcttctgtacagtcatgggttgcttggtgacaggaatatgtcctgagaaatgtgtcattcggcaatttcaaggttgtgcaagtatcaaagagtgtacttacacaaacctagatggtatagcctactacatacctaggctatatggtatagcctgttggtcctaggctacgagcctgtacagcatgttactgtactgaatattgcaggcaattaaaacacaatggtaagtatttgtctatctacccagagctaaacatagaaaaggtaaggttaaaaaaatacgagtatcggcctggtgcagtggctcacacctgtaatcccagcactttgggaggccaaggcaggtggatcacgaggtcaggagattgagaccattctggctaacacggtgaaaccctgtctctactaaaaataccg

SEQ ID NO: 230 - DNA, 507 bp, Homo sapiens
ctgcttgatgtctcctctggctttaaaaacccacatttgagtttcctccttccctctactgccctgtgcccatgcctgcccacctctcgttcttttgggcttctgacacctcccctcctccccactcctggcacctggctctgtgatacagaccgtctgcactgaatctgtgtgtaggtgtcccgcatgagatgtctgcagaatgagacttacagtgcgctgagccctggcaaaagtgaggacgttgtgcttcgatggagccaggagttcagcaccttgactctaggccagttcggatgagctggcgtctattctgcccacaccccagcccaacctgcccacgttctctattgttttgagaccccattgctttcaggctgccccttctgggtctgttactcggcccctactcacatttccttgggttggagcaacagtcccagagagggccatggtgggagctgcgccctccttaaaagatgactttacataaaatgttgatcttcg

SEQ ID NO: 231 - DNA, 910 bp, Homo sapiens
ctgcttgatgacttagggaaagatgagttgtgagagccacaacagaggcttcagaccattttcttttcttgggagccagtggatttttccagcagtgaagggacattctctacactcagatgactctaccagtggccttttaaccaagaagtagtacttgattggtccattttgaaaaccactgcaccagtgaacttttggcatctcaagaaaacattgaaaaattctatgaattgttgtagccggtgaattgagtcgtattctgtcacataatattttgaagaaaactttggctgtcgaaacatttttctctctgactgctgcttgaatgttcttgaaggctgtttcttatgtatgggttttttttaatgtgatcccttcatttgaatattaatggctttttccattaaagaataaaatattttggacaatgccgataaatgtatgaagttagtatccacatcataaattcagagtgatgtttagcagtaaatcaatattttgaagtgatacacagatgtctttcctccccacaaacttttttaaacaaaaaacaagacctcttttctttagatggtgccacctatgcccaccacaacagagattttacatggaaaccgggctcagtgagaactgatttcctgcccaatatttgtctttgggctgtctctagtgactaattattaaggaatctagctgggtatacagttcaaggctttctatggtggtaaatgaacctcaaaaatagcccgttaagacatggaaaatccagcagcaggttacccaatgcgaacaaggtaggttcgcatttatgtaaaacattcagaaaatgaagttttgaatttgtcggaacattcaaaggacttgagagcattttattgtaacttaaaaaaataaatacaactgtcac

SEQ ID NO: 232 - DNA, 786 bp, Homo sapiens
ctgcttgatggggatgacattgaatctataaattaccttgggcagtatggccattttcacgatattgattcttccctacccattgagcatgaaatgttcttctatttgtttgtatcctcttttatttccttgagcagcggtttgtagttctccttgaagaggtccttcacatcccttgtaagttggattcctaggtattttattctctttgaagcaattgtgaatgggagttcactcatgatttggctctctgtttgtctgttgttggtgtataagaatgcttgtgatttttgtacattgattttgtatcctgagactttgctgaagttgcttatcagcttaaggagattttgggctgagacaatggggttttctagatatacagtcatgtcgtctgcaaacagggacaatttgacttcctcttttcctaattgaataacctggatcccttccttacaccttatacaaaaatcaattcaagatggattaaagacttaaacgttagacctaaaaccataaaaaccctagaagaaaacctaggcattaccattcgggacataggcatgggcaaggacttcatgtctaaaacaccaaaagcaatggcaacaaaagccaaaattgacaaatgggatctaattaaactaaagagcttctgcaccggcaaaagaaactaccatcagagtgaacaggcaacctacaaaatgggagaaaattttccgcaacctactcatcttgacaaagggctaatatcaagaatctacgatgaactcaaaccaaatttacccc

SEQ ID NO: 233 - DNA, 663 bp, Homo sapiens
ctgcttgatgccaatacaaactcgacagtagttccaaatagccagaaaatggcactcccattttttccatcctacaaggtctaaataattcttgtcgtaaaataggcaaatagtctgaggtgcctgatattcaggcactcttttacacagcagcccctctctagtctctgttcccaatgcaactagtcccaaatcttccttctttccctgctgcctgtcctttctgtcccaaccccaagcattgctgagtctttctaatcttccttttctacagacccatctgagctctcacctcctcaccaggctgagctaggtcccaattcttcctcagcctccactcctccaccctataatccttttatcacctcccctcctcacacccggtctgacttacagtttctttccatgaccagccctcccccacctgcccagcaatttactcttaaaaatgtggctggagctaaaggcatagtcaaggttaatgctcctttttctttatcccaaatcagatagcatttaggctctttttcatcaaatataaaaatccagcccagttcatggctcatttggcagcaaccctgagacgctttacagccctagaccctaaaaggtcaaaagtccatctcattctcaatatacattttattacccaatctgctcccg

SEQ ID NO: 234 - DNA, 773 bp, Homo sapiens
gatccagtacataaggagttgccgcatattatatcagactgctttgagaaatctcatccctagtctattgccagttggtttctattaagcttacttgattaactcagtcctgacacccctttttggaaaatgctgatttaaacttcttaactggcaacagttggaacagtaaaccagtttgctaacatattttaaagtcttgaatgttgaagaactcatgtgatttacccttttcaactttttggaaaacgatttaatttattctaattagattaaccctattaatctatggattgggtatcaaaatgaatgccagtccagatgtgccaagacacgaaattggagctgaggactctcacgatatgcaagttcatccaacgtgaagataccataagctttttctctgaaccagagaaatgaaagtcagtttaagaggctgatagatcttggccctgttaaggcatccactttcacagttctgaaggctgagtcagccccactccacagttaggccaagaattagattttaaaacttcatctgtctgtcccagttaactgttaaataaggcctcatcctccactgaagagtatggattgaaaggattgtgaactatgtttagtgtgattgtgaacttggtgcctaatgttccatgtctgaagtttgccccagtgctacacgttggagtatacctatgtgtgtgctttgccactgaagtaagattttgcctgtatggtactgttttgtttgttaataaagtgcactgccacccccg

SEQ ID NO: 235 - DNA, 326 bp, Homo sapiens
gatcgcattgcatccaaattacaaaatgagagaagagatagaattggggaaagataatatttgaggggtaatggctgggattttttcttgaattgttgaagaacatgaaacctcaaattcaggaaacacagtgagtaccaagcaggttaaataaaaagaagacctaacattgcaattgcagaactctgaagaggaagagaagagcacaaagaggcagaaacaacagaagcctatggaataatgtcttcagagggttaagggaaaatgccttgagcctgcatatagaattattaaatatatacccagccaacccattatcctgtacg

SEQ ID NO: 236 - DNA, 578 bp, Homo sapiens
gatcgcattgctataaagaactatctgagactgggtaatttatgaagaaaaagaggtttaattgacttacagttccttaggctgtacaagaagcctggctggggagatttcaggaaatttaaaaccatggcggaaggcgaaggggaagcaggcatgtcttatatgacctgagaaggaggaggagagtgcagggggaggtgctacacacttttaaacaaccagatctcgtgagaactcactaccacaagaacagcaagggacaactctgtccccatgatccagtcacctctcaccagacccctccaacactggggattacaattttacatgagatttgggtgaagacacaaatccaaaccatgtcgccatggttaagccacacatgactgtatataacctacttgtatccttgcatatactttaaataatctctagattacacattataatacctaatacaggccgggtgtggtggcccatgcttgtaatcccagcactttcagaggccaaggcaggtggatcacgaggtcaggagttcaagaccagcctggccaacacagtgaaaccccatctctacg

SEQ ID NO: 237 - DNA, 570 bp, Homo sapiens
gatcgcattgcctgaattccaaattaatacgacaagactgcagtaatgaaaacaggatatgactggtctaaaaataggcacatagatcaatggaacagagtaaaagatccagaaataaagcaacatatttgcaggcaactgatctttgacaaagcagacaagaacgcacacttgggaaacatcactcatttcaataaatgatgctgagaaaattggattgccatatgcagaagaatgaaactggaacactatctctcacatctctcatatagaaaaataactcaggatggattaaagacttaaatgtaaaatgtgggcatatattaatacaagaagaaaacttctggacattggctgagaaaagaacttatgactaagacgtcaaagcacaggcaacaaaaatcaaattggacaaatggcacttaatttatctaaaatattttacaagcaaaataaatcaacagtgtcaacaggcaacatgcagagggaaaatatttgtaaactatgcatcatacagggaactaatatgcagagtatacaagggactcaaactactcagcaacaacaacg

SEQ ID NO: 238 - DNA, 570 bp, Homo sapiens
gatcgcattgtgaatgaaactctctatgaagacacaaagcttttgagcgccacaggggaaagtgaaaaacaattccaggaagccataattgtgtttcaagaggagtttaaatgctgtggtttggtcaatggagctgctgattggggaaataattttcaacactatcctgaattatgtgcctgtctagataagcagagaccatgccaaagctataatggaaaacaagtttacaaagagacctgtatttctttcataaaagacttcttggcaaaaaatttgattatagttattggaatatcatttggactggcagttattgaggtacagtataatcttgtattattgactcttttattcccttgcttgttcattaatttcaagaaatatttatggtgaacaaacaaacagaaaaaaaaaaccacaacaaccataaaaaacgcagatgctttagaaaagcactggatgctaaaggcccatgatctccctcgatcagcctgagtctgagggggagattccagtcttatgaagtgagcagggttgagactcttgcttctgaatgacctctctacc

SEQ ID NO: 239 - DNA, 430 bp, Homo sapiens
cttgattgccttagacaagtaagaatttatagaatctatgctctattgcactatatagaaactagaaatgcagttatactgatgtagtgcagtttgtgggaaatcaggaatggtgttctccagaatacatgaagattctcattgattgttgcaaggaatcacgaaagagatctttggtccaaagaaaacgtctctgaaagaaaatagagcatcaattaaattcaccttaaggctaagatattagttagtctattctaggttaaaaatagacagtgatttccaggatttcagttatgattattctttttaggtgactaaaatgtaaagagatgagtgggaggacttgccacagaagcaacaaagaccgagaaggtaatcgatttgtctactttccccgcatttgccattgatatcatgctatcattttacg

SEQ ID NO: 240 - DNA, 574 bp, Homo sapiens
aggtgaccgtaacattacaacctcaaacgcaggttaaagaaaataagacccaaccgccagtagcttatcaatactggccgccggctgaacttcagtatctgccacccccagaaagtcagtatggatatccaggaatgcccccagcactacagggcagggtgccatatcctcagccgcccactgtgagacttaatcctacagcatcacgtagtggacaaggtggtacactgcacgcagtcattgatgaagccagaaaacagggagatcttgaggcatggcggttcctggtaattttacaactggtacaggccggggaagagactcaagtaggagcgcctgcccgagctgagactagatgtgaacctttcaccatgaaaatgttaaaagatataaaggaaggagttaaacaatatggatccaactccccttatataagaacattattagattccattgctcatggaaatagacttactccttatgactgggaaagtttggccaaatcttccctttcatcctctcagtatctacagtttaaaacctggtggattgatggagtacaagaacaggtacg

SEQ ID NO: 241 - DNA, 218 bp, Homo sapiens
aggtgaccgtcacgtaagtggaatcatacagtctatagcctcttcagattggcttctttcacttagcaacatgccttttcatggcttgctagcttatttctttttagcactagttaatattctattgtctggatgttccacagtttatctctttatctgctgaaagataccgtggttgcttccaaatttggggaattgtgaataaacttgctatgtgc

SEQ ID NO: 242 - DNA, 408 bp, Homo sapiens
gttttcgcagcggaccgaagagaagaaaagtaggccagagccgaactctcttcctgccaagatgtctattggtgtgccgattaaagtactgcgtgaggccgagggccacattgtgacatgtgagacgaacaccggtgaggtatatcgggggaagctcattgaagcagaggacaacatgaactgccagtggccgcaagaggaagaggacgtggaatgggacgtggaaacatctttcaaaagcgaagataattttctaagttgaacagaactttgtccttttttctttcaggttatctgagttcattggagtgggtgcttgtgcatatatgctaggtatcttttgccatctttctctttagatcaggggaaatgtttaagctaaataaatctggggggttttttgttccc

SEQ ID NO: 243 - DNA, 255 bp, Homo sapiens
gttttcgcagcgagagcgaggaggaggtgctactcgtgagcagtagtcgccatccagacagatggattgtccctggaggaggcatggagcccgaggaggggccaagtgtggcagcagttcgtgaagtctgtgaggaggtatgcatggagaagagagggcacagctgtgcctcgtcatcatgaacctgtcctatctgtgtagttctttaatatgtcagcactcactgaataaatatacagtcacagaaaaagggcg

SEQ ID NO: 244 - DNA, 145 bp, Homo sapiens
gttttcgcagtcctgaacaaaatactaacaaactgaactcagcaatatatgaaatggattatatgccatgaaaagtaggatttatttaaggaacggcaaggtcggttcttcatacaaaaatcaatttattacaccatattaatag

SEQ ID NO: 245 - DNA, 307 bp, Homo sapiens
gttttcgcagcaacatttattaaaaagactatcttttccccgttgaatggtcttggcatccttgtcaaaatcatttgaccatatatgccaggatttatttctgggctgtcaactctattccaatagtctgtatgtctgtcttcctgccagtaccacattgttgtgattactgcagtttgttagtaagtcttgaaatcaggaagtgtgaaccctgaatctgtttttctttttcaacatggtgttggctatctggggtccgtggagattccatatgaatttcagcatgtattttctatttctgcgaaag

SEQ ID NO: 246 - DNA, 882 bp, Homo sapiens
gttttcgcaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtaggctggtggcaggtgatgggcctgccgtggggcaggctccctcggggcccccgtctcctgacttcctttcccctcaccaggtgacaggccgcttgcgggctcttgtgctggtacgccttaatccctgcacccaggggcactggcatcgtctccggcacctgtgcctaagaagctgctcatgatggctggtatcgatgacttgcaacacctcagcccggggctgcactgccaccctgggcaacttcggtaggtggtccacacatggggcatagccatggtctctcagctccgcttaaccacacgggtccagtgtgtgcttggcgtgttttcagggaggcagagaaaggcttcttctaatgcacgacagacccgcccagaatggcctctctgttcctaggagtgcgacaattttttggggttgggggaacttgccttaaagcacaccactgacccttcctggggggttcttttgtttttgcaacccaaggccaccttttgattgccatttctaagacctacagctaccttgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaattaagcgcg

SEQ ID NO: 247 - DNA, 681 bp, Homo sapiens
gttttcgcagtcctgaaccaaaatactaacaaactgaactcagcaatatatgaaatggattatatgccatgaaaagtaggatttatttaaggaacgggcaaggtcggttcttcatacaaaaatcaatttattacaccatattaatagaaaaaaggacaaaaactgcgtgatcatctgaataaacacaaaaagtatttgacaaattccagcctccttttgtgattaaaaaagaaaacactcagcagactgggaatagaaggaaatttcttgcagctgataaaaggcatctacaaaaaccatagctaacagtgtagttaatggcaaaagacttagtgcttttctcctaagatcaggaacaagatgaggattctgctctcaccatttctgttcaacattgtactggaggtcataaccaatgaaatcaggcaacataaggaaatgaaagtcatttagattggaaggaagaaataaaactatctctatttgcagctgacatgatcttatatttagaaaatagtaaggaatctacagaaagcaattagagctgataaatgagttcatcaagtttgcagggatacaagatcaatacacggaagtcatgtttatgtacactaacaatgaatctgaaaataaaattaacaattttcatttacagtagcataaaaggaataaatacatacg

SEQ ID NO: 248 - DNA, 143 bp, Homo sapiens
gttttcgcagtcctgaacaaaatactaacaaactgaactcagcaatatatgaaatggattatatgccatgaaaagtaggatttatttaaggaacggcaaggtcggttcttcatacaaaaatcaatttattacaccatattaac

SEQ ID NO: 249 - DNA, 658 bp, Homo sapiens
  variation 415: n is a or c or g or t.
gttgcggatccttcagagtctgttaaggaatgataaaacatcagttaagatatctggtttctgatagaactcaaaaagaaaagttactgtttcagctagtagcatatgttgaaacacaatgtaacattaaaaaaataatccaatgaagtttatggtttgttcattcctttttcagagaaactccagaaaatcttgccaaacttctgaccaggatgtatttaaaatcagaagtcataggtgatgggaatcagattgagattgaaatccctccaaccagagctgacattatccatgcatgtgatattgtagaagatgcagctattgcttatggatataacaacattcagatgactctcccgaaaacttacaccatagctaatcaatttcctcttaataagctcactgaacttctccnacatgacatggcagccgctggcttcactgaagcacttacctttgccctggtatgtcttacatgctttttctgcccagatgattatttacttagactctctgaattatttattctatgatgctttataagaatttataaaatctagactgttagaacttccagattctggaagaataactttgttacttgagattatacaattcctggtaaagtaatgttgaagcttttgtttttacagccacg

SEQ ID NO: 250 - DNA, 380 bp, Homo sapiens
gatctgacaccaagcacaggcaacagaagtgaaaataggcaaatgggactatatcagatgtaaaatgtttgtgccttaaataacacaacaattgacagaatgcaaagacaacctacggaataagagaaaatatttgcaaatgatacatctgatatgtggttaatattgaaaatatatacagaactacaacaacttaacaccaaaaaaatcaaataacacaattcaaaaatgggcaaagagcttgaatagacacgtctccaaagaagatatacaaatgacaaacaagcacgtgaaaaaatgttcattattaatcattaaagaaaagcaatttgaaaccgcaatgagataccaactcatacctatcgggatggctactatcg

SEQ ID NO: 251 - DNA, 234 bp, Homo sapiens
gatctgacaccatgaagatgactctctgatgctataagttttgaaatatttactatagggtttgttcccacccatactagatcaaggtaattggcagagcaagcaagattaacagtgacagcttggtgtctggttgggccttagagtagcctctatacccaacaaaggcaaaagaggtgtttccttggagggggcagttgactaacacaatagaaagtaagtattgcaataacg

SEQ ID NO: 252 - DNA, 230 bp, Homo sapiens
gatctgacacaaaagcacaagcaacaaaagaaaaaatagatacattgaacctcatcaaaattaaaaaccttttatgcatcaaaggacacaatcaagagagtaaaaatatgggctaggcacagtggcccaggcctctaatcacagcactttgggaggcagaggtgggagaatcacttgaggtcaggagtttgagaccaggctgggcaacatagcaagaccctgtctctacg

SEQ ID NO: 253 - DNA, 327 bp, Homo sapiens
ctgatccatgctatagtgtggatgaacttcaaaaacatgacagccaatgaaagaaaccagcaaaaagaccacaaactatgattccaggtacatgaaatgtccacaacagggaaatctatgtagacacaaagcagatttgtggctgtcttgggcttggggtttgggaagtgactgctgatgggtattggtttctttttggggtgataaaaatgttctggaattagatagtggtagtggctgcacaactttgtggattcataaccttgtgaatatattaaacactgaattgcatactttgtgtggtatgtaaattataactcagagacg

SEQ ID NO: 254 - DNA, 666 bp, Homo sapiens
ctgatccatgctataaagtggacgaaccttgcaaaccttacgtgaaatgaaaaaagccaagacccaagaggtcacctatagtatgagtcaacttgtatgaaatatccagaatagagaaatccatagagacagaaagcagacactggtagttgccaggggttggagagaggaaagaatgggaagaagacttacatagtgagtaaggggttttactttggagtgatggaaatgcgttggatctagatagaagtggcggttttgcacaacgttgtgaatggactaaatgccactgaattgttcactttatgtatttattttatttttatttatttatttgtagagacggggtcttgctgcattgcccaggctgctgtcaaactcctaagctcacccaggcgcaatggctcatgcttgtaatcccagcactgggaggccaaggtgggcggatcacctgaggtcaggagttcgagaccagcatggccaacatggcgaagccccgtctctactaaaattacaacaattagtatggtgtggtggtgaacgcctgtagtcccagctacttaggaggctgaggcaggagaatcgcttgaaccccggaggccgaaggttgcagtgagccgagatcacgccactgcactctagcctgggcgacaagagcaagactcg

SEQ ID NO: 255 - DNA, 295 bp, Homo sapiens
tggattggtcgggtgtggtggctcatgcctgtaatcccagcactttgggaggcccaggtgggtggatcacctaaggtcaggagttcaagaccagcctgaccaacatggagaaaccccatctctactaaaaaaaaaaaaaaaaaaaaaagctgtgcatggtggtgcatccctgtaatcccagctcctcgggaggctgaggcaggagaatcgcttaaaccagggagccagagattgcagtgaccaagatcacatcattgcactccagcctgggcaacaagagcgaaactccatctcg

SEQ ID NO: 256 - DNA, 389 bp, Homo sapiens
  variation 298: n is a or c or g or t.
tggattggtccctggtgccttatttagtttatttggtaaggtcatgttttcctggatagtcttgatatttatggattttcatctgtgtctgggcattgaggaataaggtatttattgttatctttgcagtagggacttgtttgtactcatctttcccaggaaggctttccagatatttcaaagaactttgatgttgtgatctaaaatatatctgtattagagaacgcaagaagcccgaagcccaggacaactttttaagtttaagaactaagtaactgtggttcttgcagacttgtanaggtactgccttggtggttttggacaacatccagaagaattctgtagattatcaggcaaaaactcttgttctcttttcttactttcttccg

SEQ ID NO: 257 - DNA, 380 bp, Homo sapiens
tggattggtcagagtggtggatagggaaaggacgcaaaccttctgagaggttggaaggttctgcagagccctgggggagaatagctgaaggtggctgttctataaccctgaggcagagggcatggagtaggtacaagggagtgtgggggaatttatcttaaacaggcttgtttacttgtgttgaccaggaactgacctttcatcatccctgtgcctgacgttccctgaaaggggaacaataagtgttaattacctacaggttgtgttggctccaggtttttggcattgtgcctagactgaataaaagcaagcagctccagcttcttggggctgctttctggccactagagccaggcagtcacctagttgctgttacaccg

SEQ ID NO: 258 - DNA, 433 bp, Homo sapiens
gatcaatcgcatcatcacccaagctgtcaaaaatgtgaacattcgtggatttttttctatttgcaagccaaaaaatgaagactgtcaacttcatggatattagaagaaggcatgagattcctaggcctgatgaaaaatatttgatggttcatcacacaaatggaagcaagagctccatatctgtatctgttccccacgtccactccaagtgacagggatgatcaaagagacccaactggatgctttctgctcagtagattttgatcacagctgaggaatctcaaggttaagaaatcccagtgctttggcagggcacggtggctcacgcctgtaatcccagcacttttggaggccgaggcgggcagatcacgaggtcaggagattgagaccatcctggctaacacggtgaaaccccgtctctactaaaaatacg

SEQ ID NO: 259 - DNA, 604 bp, Homo sapiens
tacaacgaggctgcattctgatacaacaaaggcaagtggggatttatagccaagaagcagggtggaagtcaggcaatggatcattacttagaaggagatatcaaagataaggggattccccctaaaccaacttgataggattcttgctgaaggcaggcaaggctaataagatatgaagagtggaggatgaggaatttgaccatatatgaagggtggaggatttttgctaaacagacccagtaggattcttgctaaaactggactgtcagggccaaggactgagcctaagaatgaggcctagtcaaaaagaggactcaaggctgggcgcgatggctcacgcctgtaatcccagcactttgggaggccaaggtgggtagatcacctgaggtcagaagttcgagaccagcctggccggcatgttgaaaccccatctctactaaaaatacgaaaattggctgggtgtgatggtaggcgcctgtggtcccagctacttgagaggctgaactaggagaatcacatgaacccaggaggcgaaggttgcagtgagctgagatcacaccattgcactccagcctgggcaacaagagccgaaacttttgtctcg

SEQ ID NO: 260 - DNA, 1058 bp, Homo sapiens
gatctgactggagaaaaccacagtaaatacccggggctgtgatttttgtgactttggtcaatgggacgacaggctctgtggtagactggctgtgcttgacgtaacaatgtggtactatgccgtgtggttgccggcaaggctatgtgaagatgagaatgtgtgatttggaaaatctggatgtggaacatcacgggtgccagtggaattgtgactctgtgtgtgtataaaagtttgcaaccatgggcctatatgtctgtgggataaagagaatatgttgcgtcttcttggagtatcagctttatttaaacatttgcatcagagagttgcgatttatgtgccaaagatttttgtctttttatattgaattaaatgaattttttacaaagagaaaaatgaaaataatagtaactttcaaaaaatattaagaatttccttccactcttatattctggcaatggatctttggcaatgggtctttggcagatcaatttgaaaagctcggactgaggaaattatacttggaaagcacattaggcagtcagcccttggttcctaaataccatgctccactgaaaggagcaaggcttcatggagaaatggcagattccagagctggggaagggaaagttcagaatgaacttggaacatcttgtgcttgaaagtaaggcagtgctcaaaggaatggtggggacgtgttcaaaggacatgggaaacccgattgaaggggactttcccttggcccaacttgacatggtttgagcatcaaaataaataatgctagttatagattaaaacccattaaataacagagggaatcataattccaactgatataaattagtacgtgaaaaaaaattgaaagtttgatgagggactggatgtttacattgttgcaaagaacccctgcccccccccaaatcccttaaaaatgtgtcaatataaggctgggcacggtggcttatgcctataatcccagcactttgggaggccgaggtgggcggatcgtgaggtcaggagtttgagaccagcctgaccaacgtggtgaaacctgtctctac

SEQ ID NO: 261 - DNA, 952 bp, Homo sapiens
  variation 285: n is a or c or g or t.
ttttggctcccatggtcatattctagcctcttgttatctctatagaaatgcctcttccagttctttgcccatttttgaatcaggttggtttttgttgagttttaggagttttctatatattctgggtattaatcctttatcagatatgtgatttccaagtattttatgttgggttgcctttttactgttgataatgtcctttgatgcacaagtttttaattatgaagtccaatttgtctacgttgttgttgttgttccctgctcactcttttgggttttaaaaanccaagaaaatcccctgccaaaacccaatgccatgaggctttccttttttttccctttagagacagggtctcactctgtggcctaagctggagcacagtgacgtgatcatggctcacctgcagccttgaactcctaggctcaagtgatcctcccacctcagcctcctgtgtagctgggactacaggtgtgtgccattaatttaaaaaaatttttttatagagatgggggtctcactatgctgcccagtctagtctttgaacctcctgggctcaagcaatcctccccaccttggcctcccaaaagtgctggggattacagggtattaaagccaccacatgctcctcttgaggttttatagttttagttctgatgcttaggtctttggcccactttgagttttgcatatggtattaggtaagggtccaacttcattctttttgtatgtggctctctagtttacccagaaccatttgttgataaaactgccttttcccccactgaatggtcttggcacccctgttgcatattatctgaccacacatgcaagggtttatttctgggatctgtattctattccattggtctatatgtctgtcttttatgccagtactgcactgctttgattaatgtagctttgtaataacctgaaataaaaaaatgtgagcct

SEQ ID NO: 262 - DNA, 518 bp, Homo sapiens
gatctcgacaccctaaagtggagtagaagtagtagttgaatgtattatgatctgcattagttaaatgagaggcttcatcttccttaacaaatcaatcacatcaagaacttaaacgaaaactatgtacattctgactcgaccgtaggtatgagatttttgaaattactccatagttgtcccttattatttatcctgatattaatatttaatcagaatttcgttgtaaaaagtgtatgggatttttgtatcatttttatttgtttagcatactctgaagaaatcaaagtaatagttatttaattgtggggatagggtctgaattctcataagtggattaaaattgtaagctcatttattggatgaaaggatgaaacaaaatccagaaaatgtttacttaatgttcagaataggcaagttgttaacctagctattcctatttaacaaacatttctagaaagttagaattaagattacaatattggctgggtctggtggctcatgcctgtaattccagcacc

SEQ ID NO: 263 - DNA, 858 bp, Homo sapiens
caaacgtcgggactagggcaaaggggcaatctaaagaccgaacgccgtatgtttgagattgtgagaagtctcgttcccctacagtttacttggtaaaaatggtaaaacaattctactttgtagctcgtgaatgtgaaaaattgaattaacctgttggcacacactttatcttaccagacgggtctttaatgtgtgtgtgtgtgtgtgtgtttgtgcgtgtgtgtgtgtgtgtgtgtgtgtgtgttaagtctacagggacaagaaaggttgcagaaacatttgagctcttaaagcctttttgtgtaacttggtaattatagcaactatccttatttttatatccttgattgattttaaatgtgacaaaaaatgcgcagctgtaaaaactggattttgtgtgtgaccaaatctgttctttaatttaggcttttcaaattttttccattgtcctccccacttctctttctctctttttctatcccttctgccctatacaggaactggccaagtacttcttggcagagctgctgtctgaacccaaccagacggagaatgatgccctggaacctgaagatctgtcccaggctgctgagcaggatgaaatgaggcttgagctgcagagatctgctaactcaaacccggctatggcaccccgagaacgcaaagctggctgcaagaatttcttctggaagactttcacatcctgttagctttcttaactagtattgtccatatcagacctctgatccctcgcccccacaccccatctctcttccctaatcctccaagtcttcagcgagacccttgcattagaaactgaaaactgtaaatacaaaataaaattatggtgaaattacg

SEQ ID NO: 264 - DNA, 134 bp, Homo sapiens
ctttctacccggcctcaacctttcccgccttctcaggaacatattcatttttcctcttcctcttctctaccctcaactgtctctctgctatgtttttccatcaacatataaacatgttttaggaaagctcatcg